(19) United States
(12) Patent Application Publication
(10) Pub. No.: US 2019/0352644 A1
(43) Pub. Date: Nov. 21, 2019

(54) INHIBITION OF MITOCHONDRIAL HYPOXIC STRESS INDUCED RNA EDITING BY APOBEC3G CYTIDINE DEAMINASE

(71) Applicant: Health Research, Inc., Buffalo, NY (US)

(72) Inventors: Bora E. Baysal, Orchard Park, NY (US); Shraddha Sharma, Williamsville, NY (US); Santosh K. Patnaik, Buffalo, NY (US)

(21) Appl. No.: 16/532,233

(22) Filed: Aug. 5, 2019

Related U.S. Application Data
(63) Continuation-in-part of application No. 15/564,984, filed on Oct. 6, 2017.
(60) Provisional application No. 62/714,269, filed on Aug. 3, 2018.
(60) Provisional application No. 62/145,056, filed on Apr. 9, 2015.

(51) Int. Cl. C12N 15/113; A61K 31/7105; C12Q 1/6883

(52) U.S. Cl. CPC C12N 15/1135; A61K 31/7105; C12Q 2600/136; C12Q 2600/156; C12Q 1/6883

(57) ABSTRACT

Provided are methods for inhibiting cancer cell growth comprising contacting the cancer cell with an agent which inhibits the expression of the gene, or the activity of, apolipoprotein B editing catalytic 3G (APOBEC3G). Also provided are methods for identifying agents which can induce or inhibit C>U deamination in RNA driven by apolipoprotein B editing catalytic proteins. The method comprises contacting APOBEC3G with a suitable RNA substrate and determining the extent of C>U deamination under conditions which induce APOBEC driven C>U deamination.

### CROSS-REFERENCE TO RELATED APPLICATIONS

This application claims priority to U.S. Provisional Application No. 62/714,269, filed on Aug. 3, 2018, and is a continuation-in-part of U.S. Non-provisional application Ser. No. 15/564,984, which is a National Phase of International Patent Application No. PCT/US2016/026911, filed on Apr. 11, 2016, which claims priority to U.S. Provisional Application No. 62/145,056, filed on Apr. 9, 2015, the disclosures of all of which are incorporated herein by reference.

### FIELD OF THE DISCLOSURE

This disclosure relates generally to the field of RNA editing and particularly to C>U deamination by apolipoprotein B editing catalytic (APOBEC) proteins.

### BACKGROUND OF THE INVENTION

RNA editing is a co- or post-transcriptional process that alters transcript sequences without any change in the encoding DNA sequence. Although various types of RNA editing have been observed in single cell organisms to mammals, base modifications by deamination of adenine to inosine (A>I), or cytidine to uracil (C>U) are the major types of RNA editing in higher eukaryotes. I and U are read as guanosine (G) and thymine (T) respectively by the cellular machinery during mRNA translation and reverse transcription. RNA editing can therefore alter amino acid sequences, thereby modifying and diversifying protein functions. Aberrant RNA editing is linked to neuropsychiatric diseases such as epilepsy and schizophrenia, and chronic diseases such as cancer.

RNA-dependent ADAR1, ADAR2 and ADAR3 adenosine deaminases, and APOBEC1 cytidine deaminase are the only known RNA editing enzymes in mammals. RNA sequencing studies suggest that A>I RNA editing affects hundreds of thousands of sites, though most of A>I RNA edits occur at a low level and in non-coding intronic and untranslated regions, especially in the context of specific sequences such as Alu elements. A>I editing of protein-coding RNA sequences at a high level (>20%) is rare and thought to occur predominantly in the brain. Unlike A>I editing catalyzed by adenosine deaminases⁵, the prevalence and level of C>U RNA editing in different types of cells, and its enzymatic basis and regulation are poorly understood. The activation-induced deaminase (AID), apolipoprotein B editing catalytic polypeptide-like (APOBEC) family, and cytidine deaminase (CDA) proteins of mammals harbor the cytidine deaminase motif for hydrolytic deamination of C to U. CDA is involved in the pyrimidine salvaging pathway. While AID causes C>U deamination of DNA, multiple studies have failed to identify any RNA editing activity for this protein. Humans have 10 APOBEC genes (APOBEC1, 2, 3A-D, 3F-H and 4). APOBEC3 proteins can deaminate cytidines in single-stranded (ss) DNA, and although the APOBEC proteins bind RNA, C>U deamination of RNA is known for only APOBEC1, with apolipoprotein B (APOB) mRNA as its physiological target. C>U RNA editing alters hundreds of cytidines in chloroplasts and mitochondria of flowering plants, but the underlying deaminating enzymes are unknown. The extent, regulation and enzymatic basis of RNA editing by cytidine deamination are incompletely understood.

### SUMMARY OF THE DISCLOSURE

In this disclosure, we demonstrate that the APOBEC3G (A3G) cytidine deaminase induces site-specific C-to-U RNA editing in natural killer (NK), CD8+ T cells and lymphoma cell lines upon cellular crowding and hypoxia. RNASeq analysis of hypoxic NK cells reveals widespread C-to-U recoding mRNA editing that is enriched for genes involved in mRNA translation. A3G promotes Warburg-like metabolic remodeling and reduces proliferation of HuT78 T cells under similar conditions. Hypoxia-induced RNA editing by A3G can be mimicked by the inhibition of mitochondrial respiration, and occurs independently of HIF-1α. Thus, A3G is an endogenous RNA editing enzyme, which is induced by mitochondrial hypoxic stress to promote adaptation in lymphocytes.

In this disclosure, we also demonstrate transcripts of hundreds of genes undergo site-specific C>U RNA editing in macrophages during M1 polarization and in monocytes in response to hypoxia and/or interferons. This editing alters the amino acid sequences for scores of proteins, including many that are involved in pathogenesis of viral diseases. APOBEC3A, which is known to deaminate cytidines of single-stranded DNA and to inhibit viruses and retrotransposons, mediates this RNA editing. Amino acid residues of APOBEC3A (also referred to herein as A3A or C3A) that are known to be required for its DNA deamination and antiretrotransposition activities were also found to affect its RNA deamination activity. Our study demonstrates the cellular RNA editing activity of a member of the APOBEC3 family of innate restriction factors and expands the understanding of C>U RNA editing in mammals.

Based on the present findings, this disclosure provides compositions and methods for identifying agents that can affect (inhibit or enhance) the C to U deamination of RNA by APOBEC3A. We also demonstrate that APOBEC3G (also referred to herein as A3G or C3G) is also capable of C>U RNA editing and provide compositions and methods for identifying agents that can affect (inhibit or enhance) the C to U deamination of RNA by APOBEC3G.

This disclosure also provides a method of inhibiting the growth of cancer cells comprising contacting a cancer cell, such as a lymphoma cell, with an agent that inhibits the expression or activity of A3G. In one embodiment, the disclosure provides a method for treating cancer in a subject by administering to a subject in need of treatment a composition comprising, or consisting essentially of, one or more agents that inhibit the expression or activity of A3G.

Based on the present findings, this disclosure provides a method for identifying agents that enhance or inhibit C>U deamination in a RNA substrate comprising providing a RNA substrate which contains a motif that contains a C that can undergo deamination to U; contacting the RNA substrate with a apolipoprotein B editing catalytic (APOBEC) protein (such as APOBEC3A or APOBEC3G) in the presence or absence of test agents under conditions such that C>U deamination occurs; and determining the extent of C>U deamination and identifying agents in the presence of which either an increase or decrease of deamination is observed as compared to deamination in the absence of the agent. The assay can be done in in vitro systems using purified APOBEC proteins or using cell lysates.

The disclosure also provides a method for identifying agents that enhance or inhibit C>U deamination in a RNA molecule comprising providing cells which express or overexpress APOBEC3A or APOBEC3G; in the presence or absence of test agents, optionally exposing the cells to conditions (such as hypoxia and/or interferons) under which the cells will carry out APOBEC3A driven C>U deamination of RNA or APOBEC3G driven C>U deamination of RNA; and determining the extent of C>U deamination in RNA to identify agents that induce or inhibit C>U deamination in RNA.

### BRIEF DESCRIPTION OF THE FIGURES

FIG. 1. SDHB c.136C>U RNA editing in interferon-treated monocyte enriched peripheral blood mononuclear cells (MEPs) and M1 macrophages (a) Mean and its standard error (n=3) are shown on left for editing levels in MEPs optionally treated with IFN1 (600 U per ml), IFNγ (200 U per ml) and hypoxia (1% 02) for 24 hours. The additive induction of SDHB c.136C>U RNA editing by the interferons and hypoxia is also depicted on right. Matched MEPs of seven individuals were cultured under normoxia or hypoxia with 0, 300 or 1,500 U per ml IFN1 for 24 hours. Mean and its standard error (n=7) for editing levels in the cells are shown. Editing level in cells treated with both hypoxia and IFN1 was higher than in cells treated with only hypoxia or IFN1 (Wilcoxon test P<0.02, for both concentrations of IFN1). (b) M1 and M2 macrophages were generated from unpolarized MO macrophages derived from CD14+ monocytes isolated from peripheral blood of three individuals. Mean and range (n=3) of expression of genes for markers of M1 and M2 polarization and SDHB c.136C>U editing levels in the cells are depicted. Gene expression was quantified by RT-PCR and normalized to that of ACTB.

FIG. 2. RNA editing in MEPs and macrophages (a) Mean and range of RNA editing levels (%) at sites identified by comparing transcriptome sequences of three pairs of hypoxic and normoxic MEPs, or M1 and M2 macrophages for differential RNA editing under hypoxia or M1 polarization. (b) Cumulative frequency plots of mean editing levels and fold-change effects of hypoxia or M1 polarization on editing level, by type of RNA editing. Fold-change values were estimated with the inverted beta-binomial test and their absolute values are capped at 10⁴. (c) Distributions for editing sites in coding RNAs of gene feature and effect of editing on amino acid coding, by type of RNA editing. (d) Logos indicating sequence conservation and nucleotide frequency for sequences bearing C>U editing sites (at position 0) with a higher editing level in hypoxic compared to normoxic MEPs (n=206) or M1 compared to M2 macrophages (n=122); mean and 95% CI of relative entropy values are also plotted. (e) Stem-loop structure in SDHB RNA with the c.136C>U editing site underlined and 5 b palindromes forming the stem indicated. Histograms depict the distributions of flanking palindrome length by sequence at −3 to 0 positions for the sites whose sequence logos are shown in d. (f) Effect of hypoxia or M1 polarization on transcript levels of genes that are expressed in MEPs or macrophages and code for ADAR and cytidine deaminase enzymes and some markers of M1 (FCER1A, MRC1) or M2 (CCL2, IL8) macrophage polarization. Mean and range (n=3) are shown; ns, not significant (FDR≥0.05, edgeR likelihood ratio test); #, not expressed; genes not marked ns or # are differentially expressed with FDR<0.05.

FIG. 3. C>U RNA editing induced in MEPs and monocytes by hypoxia and IFN1. (a) Site-specific C>U RNA editing for 19 genes of MEPs of three individuals was quantified by Sanger sequencing of RT-PCR products. MEPs were optionally treated with hypoxia and/or 600 U per ml IFN1 for 24 hours. (b) Editing of the sites was also similarly examined in hypoxia- and IFN1-treated MEPs of another three individuals, and in lymphocytes and CD14+ monocytes isolated from the MEPs. Because of absent or low gene expression, a C1QA RT-PCR product could not be obtained for any of the three lymphocyte isolates. Site-specific C>U RNA editing in the monocytes and lymphocytes for 12 other genes is depicted in FIG. 9b. Mean and its standard error (n=3) are shown in both panels. The detection limit for editing (5% level) is indicated. Samples without detectable editing were assigned a value of 3.8%. (c) SDHB and SIN3A protein levels in whole cell lysates (20 protein) of monocytes isolated from normoxic and hypoxic MEPs of a separate set of three donors. Non-specific signals of the Western blots are indicated by asterisk (*).

FIG. 4. Association of APOBEC3A gene expression with SDHB c.136C>U RNA editing in tumor samples of the Cancer Genome Atlas (a) C>U RNA editing was estimated from RNA sequencing data for primary head and neck squamous cell carcinoma (HNSC, n=298) and lung adenocarcinoma (LUAD, n=220), and secondary skin cutaneous melanoma (SKCM, n=187) tumors. Editing levels at all 213 C-bearing positions along SDHB ORF are plotted for every tumor. Mean levels at the positions (black), the c.136C site (red), and known C/T single nucleotide polymorphism sites (green) are indicated. Inset shows SDHB c.136C>U editing levels, and their mean and standard deviation for tumors identified as positive for the editing. (b) Tukey plots of expression of some APOBEC3 (A3), and hypoxia-(LDHA, PGK1) and macrophage-associated (CD14, MRC1) genes among SDHB c.136C>U editing-positive and -negative tumors. Error bars denote 25th percentile−1.5× inter-quartile range (IQR) and 75th percentile+1.5× IQR values. Group-sizes are noted in the legend. * FDR <0.05 (edgeR exact test for differential expression). In (b), for each set from left to right are shown for C>U− and +, HNSC, LUAD, and SKCM.

FIG. 5. APOBEC3A induces C>U RNA editing in 293T transfectants. (a) Immunoblots showing APOBEC3A (A3A), APOBEC3G (A3G) and CDA proteins in whole cell lysates (20 μg protein) of 293T cells transiently transfected with an empty vector (CO., control) or DNA constructs for expression of A3A, A3G or CDA proteins. (b) SDHB c.136C>U RNA editing in the 293T transfectants, which were optionally treated with hypoxia and/or 600 U per ml type I interferon (IFN1). Mean and range for n=3 are shown. (c) Estimation of site-specific C>U RNA editing by Sanger sequencing of RT-PCR products for 30 genes in the transfectants (n=1). The detection limit for editing (5% level) is indicated. Samples without detectable editing were assigned a value of 3.8%. Chromatograms of good quality could not be obtained for C1QA and TMEM179B for the A3G and CDA transfectants, and for the GPR160 site for the normoxic A3A transfectant. (d) Chromatograms of genomic DNA (gDNA) and cDNA PCR products of normoxic A3A transfectants indicating C>U RNA editing without C>T genomic change at positions marked with * for ASCC2, SDHB and TMEM109. Immunoblots showing ASCC2, SDHB and TMEM109 proteins in whole cell lysates (20 μg protein) of control or A3A transfectants on the right indicate reduced protein expression in association with A3A-induced stop codons in RNA. Only a single band of signal, which corresponded to a protein of full length, was seen in all three immunoblots.

FIG. 6. Knock-down of APOBEC3A (A3A) reduces C>U RNA editing in M1 macrophages (a) A3A and APOBEC3G (A3G) gene expression in M1 macrophages that were transfected with a non-specific (Ctrl.) or either one (1, 2) or equimolar mix (1+2) of two A3A-specific siRNAs at 100 nM concentration. Gene expression measurements are normalized to that for ACTB. (b) Immunoblot for A3A protein (23 kDa) of whole cell lysates (10 μg protein) of two of each set of three replicate transfectants. Non-specific signals are indicated by an asterisk (*). The signal for calnexin, a house-keeping protein, indicates total protein. (c) SDHB c.136C>U RNA editing levels in the siRNA transfectants which are determined by RT-qPCR. (d) Sanger sequence chromatogram traces of amplified cDNA fragments indicating reduced site-specific RNA editing for five other genes in A3A-specific siRNA 1 compared to Ctrl. transfectants. Mean and range (n=3) are shown for a and c.

FIG. 7. Activity of APOBEC3A (A3A) mutants in 293T transfectants (a) A3A protein level in whole cell lysates (20 μg protein) of cells transfected with an empty vector (Ctrl.) or expression constructs for wild-type (WT) A3A or its C101S, E72D or P134A variants. (b) Cytidine deamination activity of the transfectant lysates was examined in an in vitro reaction with a 5′ fluorescent dye-labeled ssDNA substrate of 40 bases (b). C>U deamination of the single cytidine residue of the substrate at position 23 followed by deglycosylation of the uridine and subsequent cleavage of the product at the abasic site was evaluated by electrophoresis of reactions of one hour duration on a polyacrylamide gel, whose fluorographic image is shown. (c) SDHB c.136C>U RNA editing in the transfectants. (d) Retrotransposition of a human LINE-1 element in a separate set of 293T transfectants. Retrotransposition, relative to the Ctrl. transfectant, was assessed with a luciferase reporter-based assay and is quantified as the ratio of firefly and Renilla luciferase activities. Mean and range (n=3) are shown for c and d.

FIG. 8. In vitro cytidine deamination of SDHB RNA and ssDNA by APOBEC3A (a) c.136C>U editing of an ˜1.1 kb exogenous SDHB ORF RNA by whole cell lysates of control or APOBEC3A 293T transfectants. Duration of the deamination reactions and amount of lysate protein in them are noted. For some reactions, lysates were pre-heated at 85° C. for 15 minutes. (b) c.136C>U editing of the RNA by 10 μM purified C-His₆-tagged APOBEC3A protein. The reactions had 180 amole SDHB RNA and 100 nM ZnCl₂. Reactions for b conducted for two hours at 37° C. Mean and range (n=3) are shown in a and b.

FIG. 9. C>U RNA editing by hypoxia and IFN1 in monocytes but not lymphocytes. (a) Dot plot of forward and side scatter values of a sample of MEPs cultured under hypoxia with 600 U per ml IFN1 for 24 hours, indicating the strategy used to isolate monocytes and lymphocytes from MEPs by flow cytometry based on light scattering and cell surface expression of CD14 protein. (b) Estimation of site-specific C>U RNA editing by Sanger sequencing of RT-PCR products for 12 genes in monocytes and lymphocytes isolated from hypoxic, IFN1-treated MEPs of three individuals. Editing levels for individual monocyte or lymphocyte samples and their means are shown. A level could not be calculated for FAM89B and RNH1 for some samples because of poor quality of Sanger sequencing. The detection limit for editing (5% level) is indicated. Samples without detectable editing were assigned a level of 3.8%. Information on site-specific C>U RNA editing in the samples for 19 other genes is shown in FIG. 3b.

FIG. 10. Effect of IFN1 on APOBEC3A, APOBEC3G and CDA gene expression in MEPs. MEPs of three individuals were optionally treated with 300 or 1,500 U per ml IFN1 for 24 hours under normoxia or hypoxia. SDHB c.136C>U RNA editing and expression of APOBEC3A, APOBEC3G and CDA (normalized to SDHB) were quantified by RT-PCR. Mean and range (n=3) of changes in gene expression and SDHB RNA editing relative to untreated cells are shown.

FIG. 11. SDHB c.136C>U RNA editing in 293T cells co-transfected with expression constructs for APOBEC3A and SDHB open reading frames. 293T cells were transiently transfected with the plasmid DNAs. An empty vector was used for cells that did not receive the APOBEC3A plasmid. Reverse transcription reactions were performed with or without reverse transcriptase (RT), and the products were used as template in allele-specific PCR to quantify editing of either exogenous or both exogenous and endogenous SDHB transcripts. Mean and range (n=3) are shown.

FIG. 12. Effect offreeze/thaw and cell density on SDHB c.136C>U RNA editing. (a) CD14⁺ monocytes, isolated from PBMCs of one donor using immunomagnetic beads and stored frozen in RPMI-1640 medium with 36% v/v fetal bovine serum and 10% v/v dimethyl sulfoxide at −80° C., were thawed and cultured at indicated density for an hour and then optionally treated with hypoxia for a day. Mean and range (n=3) of editing levels in normoxic and hypoxic cells are shown. Significant induction of SDHB RNA editing by hypoxia (>2-fold, compared to normoxia) is not noticeable. (b) In freshly isolated MEPs, a high level of editing under hypoxia was more consistently observed with a cell density above 20 million/ml. The data that is plotted was generated in multiple experiments that had a total of 78 cultures of MEPs isolated from a total of 33 donors. Seventy-six of the cultures were paired; i.e., cells of a specific donor and at a specific density were cultured under either hypoxia or normoxia for a day.

FIG. 13. Correlation of SDHB c.136C>U RNA editing level measurements obtained by allele-specific RT-PCR and Sanger sequencing. The scatterplot shows estimates of editing level determined by both RT-PCR and Sanger sequencing of amplified cDNA for 22 samples of normoxic or hypoxic MEPs. Values of the Pearson correlation coefficient (r) and slope (m) of the linear regression line (black; least squares fitting technique), and their 95% confidence intervals, and the line of identity (gray) are also depicted.

FIG. 14. Scans of films of immunoblotting assays whose results are shown in FIGS. 3c, 5d and 6b. (a) Cropped views of these scans are shown in FIG. 3c. For SIN3A, that figure shows only the signal at the position marked with an asterisk (*). (b) Cropped views of these scans, only for experiment (Expt.) B, are shown in FIG. 5d. (c) Cropped views of these scans are shown in FIG. 6b. That figure shows the two signals at positions marked with an asterisk (*) for lanes that are marked here as 2-3, 5-8 and 13-14. Lanes 1-3, 4-6, 7-9 and 13-15 respectively had protein lysates of the biological triplicates of CO., 1, 2 and 1 +2 siRNA transfectants. Molecular weight (MW) markers and the protein(s) being detected are noted in all three panels of the figure.

FIG. 15. Read counts in raw and processed RNA sequencing data

FIG. 16. Mapping of RNA sequencing data with the Subread subjunc aligner

FIG. 17. Mapping of RNA sequencing data with the TopHat2 aligner

FIG. 18. Number of candidate sites along different steps of analysis of pileups of Subread-aligned RNA sequencing reads for identification of differentially RNA-edited sites showing separate analyses of RNA sequencing data of MEPs and macrophages

FIG. 19. Summary of genomic feature and effect on translation codon of RNA editing at positions for which the editing level was differentially affected by hypoxia or macrophage polarization as annotated by ANNOVAR

FIG. 20. Enrichment for ontologies of genes for sites with C>U RNA editing differentially affected by hypoxia or M1 macrophage polarization. Gene set enrichment analyses, for sets with at least two genes, were performed with PANTHER 9.0

FIG. 21. Genes differentially expressed between tumor samples of the Cancer Genome Atlas (TCGA) that are positive positive or negative for SDHB c.136C>U editing in all three cancers. Only genes with absolute log₂ fold-change >0.5 were considered; genes are ordered by decreasing mean of the three log₂ fold-change values

FIG. 22. Differential expression of genes coding for known RNA editing and cytidine deaminase enzymes following hypoxia treatment of MEPs, M1 (vs. M2) macrophage polarization, or between SDHB c.136C>U editing-positive and -negative TCGA tumor samples. Log₂ fold-change expression values are shown for genes identified as differentially expressed in hypoxic vs. normoxic MEP, M1 vs. M2 macrophage, or SDHB c.136C>U editing-positive vs. -negative cancer tumor tissue comparisons; ns, statistically insignificant for differential expression (FDR≥0.05; see Methods); NE, identified as not expressed (see Methods).

FIG. 23. Sequences of DNA oligonucleotides used as PCR or sequencing primers. Oligonucleotide used as a sequencing primer is indicated with an asterisk.

FIG. 24. Sequences of DNA oligonucleotides used for site-directed mutagenesis of APOBEC3A coding sequence. Reference sequence is NCBI RefSeq NM_145699.2

FIG. 25. Transient overexpression of APOBEC3G in 293T cells induces C>U RNA editing of host genes. Sanger sequencing of selected genes confirms site-specific C>U RNA editing by overexpressing A3G. EV=transfected with empty vector, A3G=transfected with expression plasmid for A3G.

FIG. 26. Salient characteristics of C>U RNA editing by APOBEC3G in 293T cells (A) Mean and range of editing level at the 712 sites identified as targets for A3G-mediated editing are shown for the three A3G transfectant samples. The sites are ordered by the mean editing level. (B) Logo indicating sequence conservation and base frequency for sequences bearing the editing sites (at position 0). (C) Histogram of lengths in bases of inverted repeat sequences flanking the editing sites.

FIG. 27. Site-directed mutagenesis of APOBEC3G shows requirement of both N- and C-terminal domain active catalytic site residues for site-specific RNA deamination. A. Sanger sequencing of selected genes shows that mutations in in conserved catalytical sites in N-terminal (C97S) and C-terminal (C291S) markedly decrease or abolish RNA editing (edited Cs are highlighted) whereas non-catalytic site mutants W94A and W127A has limited effect on RNA editing. B. Quantification of editing frequencies in selected sites is shown in B and C. In B, the bars from left to right for each set are: ITG1, PRPSAP2, RFX7, SCD, and TM7SF3. In C, the bars from left to right for each set are: GOLGA5 (R692X), KIAA1715, and MED1. Dotted line indicates the threshold (0.048) where RNA editing levels can be confidently measured by the Sequencher software.

FIG. 28. Additional mutants confirm essential role of APOBEC3G N-terminal conserved catalytic residues for RNA editing. (A, B and C). Mutations in N-terminal conserved catalytic residues H65R, E67Q, C97S and C100S markedly diminish or abolish RNA editing, whereas mutations in Vif-binding residues D128K and P129A has no effect on RNA editing levels. In C, the bars from left to right for each set are: KIAA1715, PRPSAP2, SCD, TM7SF3.

FIG. 29. Cell specific expression of APOBEC3G (A3G) and the induction of RNA editing in NK cells. (a) Cell type specific expression of A3G (probe:214995_s_at) in Primary Cell Atlas, a meta-analysis of publicly available 100+ microarray datasets, available through the BIOGPS portal. (b) A3G gene expression in NK, CD4+ T and CD8+ T cells. Gene expression measurements are normalized to that of β2-Microglobulin (c) Immunoblot showing the protein levels of eIF-2α phosphorylated at Ser 51 in whole cell lysates of NK cells at 0, 20 and 40 h under normoxia (N) or hypoxia (H). Thapsigargin (Tg) treated NK cells are used as a positive control and β-actin is used as a loading control (d) Estimation of site-specific C>U RNA editing by Sanger sequencing of RT-PCR products for TM7SF3, RPL10A and RFX7 of NK, CD4+ T and CD8+ T cells subjected to hypoxia. See Methods for statistical analysis.

FIG. 30. Distribution of site-specific A3G-mediated mRNA editing in NK cells (a) A3G-mediated C>U RNA editing in NK cells resulting in non-synonymous changes (n=62) based in the order of highest to lowest editing level in hypoxia (40 h). Black symbols indicate genes that acquire nonsense RNA editing (n=10). (b) Sanger sequence chromatogram traces of amplified cDNA fragments comparing site-specific C>U editing in mRNAs of ten genes under normoxia and hypoxia. (c) Graph representing the editing levels of mRNA substrates of A3G in hypoxic NK cells and the location of editing in the mRNA as well as the type of change in the transcript sequence due to this editing (d) Venn diagram showing the number of unique and overlapping RNA editing sites (exonic and UTR) among hypoxic NK cells, 293T/A3A and 293T/A3G overexpression systems. (e) Logo indicating sequence conservation and nucleotide frequency for sequences bearing C>U editing sites (at position 0) among the edited transcripts in NK cells (n=122) (f) Heat map representing the most upregulated genes (n=50) in NK cells subjected to cellular crowding and hypoxia (cell stress)

FIG. 31. Distribution and induction of A3G-mediated C>U mRNA editing in lymphoma cell lines (a) A List of cell lines in the CCLE database that have the highest expression of A3G (Affymetrix). The highlighted cell lines JVM3 and HuT78 are used in this study. (b) Immunoblot showing the protein levels of eIF-2α phosphorylated at Ser 51 in whole cell lysates of scramble WT and KD HuT78 cells at various time points. Thapsigargin (Tg) treated HuT78 cells is a positive control and α-Tubulin is used as a loading control. The WT and the KD HuT78 cells samples were run on two separate gels on the same day. The dashed line separates the two gels (c) A3G and A3F gene expression in control WT and KD HuT78 cells under normoxia (N) and hypoxia (H). Gene expression measurements are normalized to that of β2-Microglobulin (d) Immunoblot for A3G protein expression in whole cells lysates of WT and KD HuT78 cells. α-Tubulin is used as a loading control (e) Graph representing the percentage site-specific C>U RNA editing level for TM7SF3, EIF3I and RFX7 of scramble WT and KD HuT78 cells in normoxia. See Methods for statistical analysis.

FIG. 32. Induction of A3G-mediated C>U mRNA editing by the inhibition of mitochondrial respiration (a) Immunoblot showing the protein level of HIF-1α in whole cell lysates of HuT78 when subjected to normoxia (N) and hypoxia (H) in 96 well (W) and 6 W plates for 24 hours. All lanes are part of the same gel. The dashed line represents the cropped region. The percentage C>U RNA editing levels in TM7SF3 under these conditions is displayed below (b) The percentage C>U RNA editing in TM7SF3 when HuT78 cells are treated with Myxothiazol (MXT), Thapsigargin (Tg), Atpenin (AtA5) and Hypoxia (H) for 24 hours (Day 1) or 42 hours (Day 2) (n=3) (c) Immunoblot showing the protein level of HIF-1α in whole cell lysates of HuT78 when subjected to normoxia (N) with or without the mitochondrial inhibitors (MXT and AtA5) and hypoxia (H) for one or two days (d) The percentage C>U RNA editing in TM7SF3 when NK-92 cells are treated with Myxothiazol (MXT), Atpenin (AtA5) and Hypoxia (H) for 42 hours (n=3). See Methods for statistical analysis.

FIG. 33. A3G-mediated C>U mRNA editing results in Warburg-like effect in lymphoma cell lines (a) Plot representing the basal respiration versus glycolysis in T0 unstressed and stressed cells (cellular crowding in normoxia) in WT and KD HuT78 cells (mean and SD, n=3-4). (b) Bar graph showing the respiration to glycolysis ratios (R/G) normalized to unstressed WT and KD HuT78 cells are shown (mean and SEM). (c) Bar graph representing the fraction of viable WT and KD HuT78 cells when subjected to cellular crowding for 24 hours in normoxia (see methods) followed by culture in non-stressed conditions for another 48 hours (Mean and SEM, n=3). See Methods for statistical analysis.

FIG. 34. Simplified diagram summarizing the induction and relevance of A3G-mediated site-specific C>U cellular mRNA editing in NK cells and lymphoma cell lines. NK/lymphoma cell is shown under normal physiological conditions when the cells are unstressed (left) or when the cells are stressed by hypoxia (top right) or due to the inhibition of mitochondrial respiration (bottom right). Under normal physiological conditions (baseline) mRNAs (stem-loop) in NK cells do not undergo C>U RNA editing. Under hypoxic stress or upon mitochondrial respiratory inhibition, an unknown signal originating in the mitochondria (red) triggers site-specific A3G-mediated C>U editing in multiple mRNA substrates bearing a stem-loop structure. The cellular mRNA editing induced by mitochondrial hypoxic stress may result in translational reprogramming of NK cells, Warburg-like metabolic remodeling by preferring glycolysis over mitochondrial respiration, and reduced cellular proliferation in order to promote adaption during NK/lymphoma cell stress.

FIG. 35. Graph representing the percentage RNA editing in TM7SF3 under normoxia, hypoxia, or hypoxia with IFN-g treatment, (a). A3G-mediated C>U RNA editing in NK cells resulting in synonymous changes (n=42) based in the order of highest to lowest editing level in hypoxia, (b).

FIG. 36. Plot representing the average RNA editing and gene expression levels (normalized values) of NK cells in hypoxia. There is no statistically significant correlation between the gene expression and RNA editing levels (r=0.1695, p=0.0620, n=122 genes, the slope of linear regression curve is not significantly different from zero).

FIG. 37. Functional clustering of genes of non-synonymously edited RNAs using the DAVID Bioinformatics resources.

FIG. 38. Heat map showing the gene expression changes in NK cells subjected to cellular crowding and hypoxia.

FIG. 39. APOBEC3G expression (Affy) in various cell lines obtained from https://portals.broadinstitute.org/ccle/page?gene=APOBEC3G.

FIG. 40. NK cell purity analysis by flow cytometry. NK cells are negative for CD3 while positive for CD56.

### DETAILED DESCRIPTION OF THE INVENTION

The present disclosure is based on our identification of an enzyme and conditions which can induce C>U deamination. This disclosure provides methods and compositions to identify agents which can affect C>U deamination. Such agents may be useful for inhibition of C>U deamination or enhancing C>U deamination.

The term “treatment” as used herein refers to reduction in one or more symptoms or features associated with the presence of the particular condition being treated. Treatment does not necessarily mean complete remission, nor does it preclude recurrence or relapses. For example, treatment of cancer, such as lymphoma, can refer to reduction of one or symptoms associated with the cancer.

The term “therapeutically effective amount” as used herein in reference to a single agent is the amount sufficient to achieve, in a single or multiple doses, the intended purpose of treatment.

Where a range of values is provided in this disclosure, it should be understood that each intervening value, to the tenth of the unit of the lower limit between the upper and lower limit of that range, and any other intervening value in that stated range is encompassed within the invention, unless clearly indicated otherwise. The upper and lower limits of these smaller ranges may independently be included in the smaller ranges encompassed within the disclosure.

The singular form used in this disclosure includes the plural form and vice versa, unless indicated otherwise.

In the present disclosure, we analyzed the cell type specific expression of APOBEC3G (A3G), and have identified widespread RNA editing mediated by A3G, induced by high cell density and hypoxia in NK, CD8+ T cells and in the representative lymphoma cell line (Hut78 T). Our findings reveal that under hypoxic stress, A3G-mediated RNA editing converges at targets involved in mRNA translation, likely to reorganize the cellular translation apparatus. Furthermore, we show that A3G promotes adaptation to hypoxic stress by suppressing cell proliferation and by promoting glycolysis over mitochondrial respiration. Thus, A3G is a novel endogenous RNA editing enzyme which can facilitate cellular adaptation to mitochondrial hypoxic cell stress in cytotoxic lymphocytes.

The disclosure provides a method for inhibiting the growth of cancer cells by inhibiting the expression or activity of A3G. The method comprises contacting the cancer cells with an agent that will inhibit the expression or activity of A3G. The agent may be a polynucleotide, a peptide, polypeptide or protein, or a small molecule. In one embodiment, the disclosure provides a method for treatment of cancer comprising administering to a subject in need of treatment a composition comprising, or consisting essentially of, an agent that can inhibit the A3G gene, or that can inhibit the activity of the A3G protein. Examples of cancers that can be treated by the method of this disclosure include, but are not limited to: hematologic neoplasms including but not limited to B cell leukemia/lymphomas, myelomas, and acute and chronic myeloid neoplasms as well as melanoma, kidney cancers, gliomas, pancreatic cancer, mesotheliomas, urinary tract and thyroid cancers.

We have found that hypoxia (such as 1% O₂) enhances the C>U editing of an exemplary RNA, e.g., SDHB RNA at c.136 in monocytes, with an editing level of ˜18% observed for monocyte-enriched PBMCs (MEPs) after 48 hours of hypoxia. Monocytes infiltrate tumors, atheromatous plaques, and sites of infection and inflammation, which are characterized by micro-environmental hypoxia. C>U RNA editing of SDHB m′ ay therefore represent a hypoxia-adaptive mechanism that may have implications for the pathogenesis of chronic inflammatory diseases.

To identify additional C>U RNA editing events in monocytes and monocyte-derived macrophages, we analyzed their whole transcriptome RNA sequences. We show that transcripts of hundreds of genes including those implicated in viral pathogenesis and Alzheimer's disease are targets of editing in monocytes and macrophages. We show that such editing is regulated by oxygen, interferons (IFN) and also during macrophage polarization. Most importantly, we demonstrate that APOBEC3A, which belongs to the APOBEC3 family of cytidine deaminases, is an RNA editing enzyme. These findings significantly expand our understanding of C>U RNA editing and open new avenues of inquiry on the role of APOBEC3 genes in viral and chronic diseases.

The subject treated with the compositions and methods of this disclosure can be a human subject or a non-human animal subject. The subject can be of any gender or age.

In one embodiment, the agent is a siRNA for use in RNA interference (RNAi) mediated silencing or downregulation of A3G mRNA. RNAi agents are commonly expressed in cells as short hairpin RNAs (shRNA). shRNA is a RNA molecule that contains a sense strand, antisense strand, and a short loop sequence between the sense and antisense fragments. shRNA is exported into the cytoplasm where it is processed by dicer into short interfering RNA (siRNA). siRNA are typically 21-23 nucleotide double-stranded RNA molecules that are recognized by the RNA-induced silencing complex (RISC). Once incorporated into RISC, siRNA facilitate cleavage and degradation of targeted mRNA. Thus, for use in RNAi mediated silencing or downregulation of A3G expression, the polynucleotide agent can be either a siRNA or a shRNA. Representative but non-limiting shRNAs for use in various aspects of the instant disclosure are provided in Example 1.

shRNA can be expressed from any suitable vector such as a recombinant viral vector either as two separate, complementary RNA molecules, or as a single RNA molecule with two complementary regions. In this regard, any viral vector capable of accepting the coding sequences for the shRNA molecule(s) to be expressed can be used. Examples of suitable vectors include but are not limited to vectors derived from adenovirus, adeno-associated virus, retroviruses (e.g, lentiviruses), rhabdoviruses, murine leukemia virus, herpes virus, and the like. A preferred virus is a lentivirus. The tropism of the viral vectors can also be modified by pseudotyping the vectors with envelope proteins or other surface antigens from other viruses. As an alternative to expression of shRNA in cells from a recombinant vector, chemically stabilized shRNA or siRNAs may also be used administered as the agent in the method of the invention. Vectors for expressing shRNA which in turn produces siRNA once introduced into a cell are commercially available. Further, shRNAs or siRNAs targeted to virtually every known human gene are also known and are commercially available. For example, we used clone ID V2LHS-80786 from Dharmacon to reduce the expression of A3G. In embodiments specific shRNAs are those which comprise polynucleotide sequences, such as described in Example 1, wherein those sequences are targeted to A3G.

In an embodiment, the agent may be an antibody that recognizes an A3G protein (an “A3G protein”). The antibodies used in the invention may bind to an A3G protein such that the binding of the antibody interferes with the activity of the A3G protein. Antibodies that recognize A3G protein for use in the invention can be polyclonal or monoclonal, or synthetic or hybrids. Methods for making polyclonal and monoclonal antibodies are well known in the art. It is expected that antigen-binding fragments of antibodies may be used in the method of the invention. Examples of suitable antibody fragments include Fab, Fab', F(ab')₂, and Fv fragments. Various techniques have been developed for the production of antibody fragments and are well known in the art. The antibodies or antigen binding fragments thereof may be humanized. Thus, the antibodies useful for the present disclosure may be chimeric, humanized or human. The term antibodies, as used herein, also includes nanobodies, diabodies and the like.

In one embodiment, AG3 inhibitors may be used as adjuvants or as agents in combination therapy to treat cancer.

Compositions comprising an agent that can suppress A3G for use in therapeutic purposes may be prepared by mixing with any suitable pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins.

If the agent is a polynucleotide, it can be administered to the individual as a naked polynucleotide, in combination with a delivery reagent, or as a recombinant plasmid or viral vector which either comprises or expresses the polynucleotide agent. Suitable delivery reagents for administration include the Mirus Transit TKO lipophilic reagent; lipofectin; lipofectamine; cellfectin; or polycations (e.g., polylysine), or liposomes.

In general, a formulation for therapeutic use according to the method of the invention comprises an amount of agent effective to suppress expression of A3G. Those skilled in the art will recognize how to formulate dosing regimens for the agents of the invention, taking into account such factors as the molecular makeup of the agent, the size and age of the individual to be treated, and the type and location of the cancer to be treated.

In an aspect, the present disclosure provides a method for identifying agents that enhance or inhibit C>U deamination in a RNA molecule comprising providing a RNA substrate which contains a motif that contains a C that can undergo deamination to U; contacting the RNA substrate with a apolipoprotein B editing catalytic (APOBEC) protein in the presence or absence of test agents; and determining the extent of C>U deamination and identifying agents in the presence of which either an increase or decrease of deamination is observed as compared to deamination in the absence of the agent, wherein the APOBEC protein is APOBEC3A or APOBEC3G. In an embodiment, the motif that comprises a C that undergoes RNA deamination to U is UC or CC where the deaminated C (underlined) is located at the 3′-end of a RNA stem-loop structure. In an embodiment, the motif is CCAUCG or CCACCG. In another embodiment, the APOBEC3A or APOBEC3G protein is a purified and/or recombinant protein. In an embodiment, the APOBEC3A or APOBEC3G protein is in a cell lysate.

In an aspect, the present disclosure provides a method for identifying agents that enhance or inhibit C>U deamination in a RNA substrate comprising providing cells which express apolipoprotein B editing catalytic 3A (APOBEC3A) or 3G (APOBEC3G); in the presence or absence of test agents, exposing the cells to conditions under which the cells can carry out APOBEC3A or APOBEC3G driven C>U deamination of RNA; and determining the extent of C>U deamination in RNA to identify agents that induce or inhibit C>U deamination in RNA, wherein an increase in C>U deamination as compared to deamination in the absence of the agent identifies an agent that enhances C>U deamination, and a decrease in C>U deamination as compared to deamination in the absence of the agent identifies an agent that inhibits C>U deamination.

In an embodiment, the cells are monocytes and/or macrophages, and the condition under which the cells carry out APOBEC3A or APOBEC3G driven C>U deamination of RNA comprises exposure to hypoxia, interferon, which can be type 1 interferon or interferon gamma, or both hypoxia and interferon.

In an aspect, this disclosure provides a method for identifying agents that enhance or inhibit C>U deamination in a RNA substrate comprising a method for identifying agents that enhance or inhibit C>U deamination in a RNA substrate comprising providing cells which have been transfected to overexpress apolipoprotein B editing catalytic3A (APOBEC3A) or apolipoprotein B editing catalytic 3G (APOBEC3G); in the presence or absence of test agents, determining the extent of C>U deamination in RNA to identify agents that enhance or inhibit C>U deamination in RNA. In an embodiment the cells are 293T cells.

By transcriptome sequencing and analysis, we show here that transient overexpression of APOBEC3G in 293T cells causes site-specific C-to-U (C>U) RNA editing in 712 sites resulting in protein recoding of 217 cellular genes. APOBEC3G-mediated RNA editing causes recoding in CHMP4B, SIN3A, subunits of mediator complex MED (MED1, MED28), NFAT5, NMT1, RBM14 and MAPK1 that are known to be involved in HIV-1 replication. Site-directed mutagenesis studies show that conserved catalytic residues in both cytidine deaminase domains of APOBEC3G are required for RNA cytosine deamination. Purified C3G enzyme catalyzes site-specific RNA editing in vitro. These results demonstrate that APOBEC3G is a C>U RNA editing enzyme that may antagonize retroviral infection by mutating the transcripts of accessory host genes.

In one embodiment, the disclosure provides a method of identifying compounds which can induce or inhibit the C>U deamination of RNA comprising providing a substrate (RNA molecule) for the deamination and an enzyme that is capable of C>U deamination under conditions such that the enzyme will catalyze the C>U deamination of the RNA. The enzyme can be APOBEC3 or APOBEC3G. The enzyme may be provided in a purified or recombinant form such that the reaction can be carried out in a cell-free system. In one embodiment, the enzyme may be provided as a component of a cell lysate. In one embodiment, the enzyme may be provided in vivo. These enzymes are available commercially (such as from Origene).

The disclosure provides a method for screening a plurality of compounds or agents for their ability to induce or inhibit APOBEC3A and/or APOBEC3G driven C>U deamination in RNA substrate comprising the motif for the C>U deamination. The method can comprise contacting purified or recombinant APOBEC3A and/or APOBEC3G protein with the RNA substrate in an in vitro system in the presence or absence of the test compounds and determining C>U deamination. The method can comprise contacting cell lysates comprising APOBEC3A and/or APOBEC3G with the RNA substrate in the presence or absence of the test compounds or agents and determining C>U deamination. Increased C>U deamination identifies compounds or agents that enhance C>U deamination. Decreased C>U deamination identifies compounds or agents that inhibit C>U deamination. Identification of increased or decreased C>U deamination can be done relative to a control, which may be run in the absence of the enzyme, substrate or in the presence of enzymes or substrates that do not support C>U deamination.

In one embodiment, the disclosure provides a method for screening a plurality of compounds for their ability to induce or inhibit APOBEC3A driven C>U deamination in RNA comprising exposing whole cells, which express APOBEC3A and which comprise an RNA substrate, to conditions that induce APOBEC3A driven C>U deamination in the presence or absence of the test compounds and determining C>U deamination. Conditions that induce APOBEC3A driven C>U deamination can be hypoxia and/or interferons. For example, for monocytes, both hypoxia and/or interferons induce APOBEC3A driven C>U deamination. For macrophoages, interferons induces APOBEC3A driven C>U deamination.

In one embodiment, the method can comprise exposing whole cells, in which APOBEC3A and/or APOBEC3G has/have been overexpressed, and which comprise an RNA substrate, to test compounds and determining C>U deamination. Overexpression of APOBEC3A and/or APOBEC3G can be carried out in any cells, such as cell lines, such as 293T cells. The cells can then be processed for determining the level of C>U deamination. Increased C>U deamination identifies compounds or agents that enhance C>U deamination. Decreased C>U deamination identifies compounds or agents that inhibit C>U deamination.

In one embodiment, the disclosure provides a method for screening a plurality of compounds for their ability to induce APOBEC3A or APOBEC3G driven C>U deamination in a RNA molecule in the presence or absence of hypoxia and/or interferons comprising one or more of the following: i) testing the plurality of compounds for increasing deamination of C in isolated DNA molecule (such as a single stranded DNA molecule); ii) testing the positive compounds from i) for an enhancing effect on C>U deamination in isolated RNA molecule (such as, for example, SDHB); and iii) testing the positive compounds for enhancing effect on C>U RNA deamination from ii) in cell based assays, and then optionally in vivo systems. In one embodiment, step ii) could be eliminated with positive compounds from i) being directly tested for enhancing C>U RNA deamination in a cell based assay.

In one embodiment, the disclosure provides a method for screening a plurality of compounds for their ability to inhibit APOBEC3 driven C>U deamination in a RNA molecule in the presence or absence of hypoxia and/or interferons comprising performing one or more of the following: i) testing the plurality of compounds for reducing deamination C in isolated DNA molecule (such as a single stranded DNA molecule); ii) testing the positive compounds from i) for reducing C>U deamination in isolated RNA molecule; and iii) optionally testing the positive compounds for reducing C>U RNA deamination from ii) in cell based assays wherein the cells are exposed to hypoxia and/or interferons, and then optionally in vivo systems. In one embodiment, step ii) could be eliminated with positive compounds from i) being directly tested for reducing C>U RNA deamination in a cell based assay.

Determination of deamination of DNA or RNA can be carried out by methods known in the art. For example, DNA and RNA deamination can be determined by Sanger reaction or high throughput sequencing techniques. For DNA deamination, treatment with UDG glycosylase and alkaline treatment may be used to cleave the DNA molecule. For RNA deamination, radioactive primer extension and gel electrophoresis may be used. In one embodiment, allele specific qPCR may be used to measure RNA editing (Baysal et al., PeerJ., Sep. 10, 2013, 1:e152. doi: 10.7717/peerj. 152. eCollection 2013); incorporated herein by reference).

Determination of C>U deamination can be carried out in a cell-free system. For example, isolated polynucleotide (polyribonucleotides or polydeoxyribonucleotides) may be used. In one embodiment, the length of the polynucleotides is at least 15. Thus, the substrates may be short polynucleotides or long RNA or single stranded DNA molecules. In one embodiment, the length is from 15 to 100 nucleotides and all integer lengths therebetween. In one embodiment, the length is from 30 to 50 nucleotides.

In vitro assays with purified APOBEC3A or APOBEC3G can be carried out by contacting APOBEC3A or APOBEC3G (such as 1-10 mM) with a suitable polynucleotide substrate (such as 2-2.5 pM full-length RNA or single-stranded DNA, in suitable buffers (such as 10 mM Tris (pH 8.0), 50 mM KCl and 10 or 100 uM ZnCl₂ with or without 10 mM 1,10-phenanthroline). The reactions can be incubated for suitable periods of time. For in vitro RNA-editing assay with transfectant cell lysate, reaction can be carried out at 37° C. for 4-11 h with RNA in a suitable buffer containing RNAse inhibitor (such as in 100 mM KCl, 10 mM HEPES (pH 7.4), 1 mM DTT and 1 mM EDTA). Cell-based assays can be performed 24-48 hours after transfection of cells (such as 293T cell line) with a mammalian expression vector containing APOBEC3A or APOBEC3G coding regions. This can be useful to achieve overexpression of the enzymes. Total RNA can be extracted and RT-qPCR is can be performed (such as by using a method described in Baysal et al. PeerJ:e152, incorporated herein by reference).

In one embodiment, the RNA substrate contains the motif CCAUCG or CCACCG with the underlined C targeted for editing. In one embodiment, variants of this motif can also be used which are single-nucleotide variations within the motif. In one embodiment, UC is the motif, and in one embodiment, CC is the motif with underlined C targeted for editing. In one embodiment, the RNA (polyribonucleotide substrate) contains stem-loop structures which contain the editable Cs in the loops. The stem loop size may be 4 nucleotides or more. In one embodiment, the RNA substrate comprises only one target C>U editing motif. In one embodiment, the RNA substrate comprises 2 or more target C>U editing motifs.

In one embodiment, the ss DNA substrate comprises the motif TC. In one embodiment, the ss DNA comprises the motif CC. The ss DNA substrate may comprise one or more target motifs.

For carrying out cell based assays, any type of cells may be used. For example, cells may be in vivo, or freshly isolated or primary or secondary cultures, or cell lines. In one embodiment, the cells are peripheral blood mononuclear cells (PBMCs). In one embodiment, the cells may be lymphocytes and monocytes. These cells may be purified from the blood by using routine methods (such as density gradients, flow cytometry and the like). The cells may be further purified as desired. For example, CD14 monocytes may be isolated using anti-CD14 antibody based methods employing magnets or flow cytometry. Alternatively, monocytes can be physically enriched by cold-aggregation of PBMCs, as described herein, or by plate adherence. We found that APOBEC3A-mediated RNA editing occurs primarily CD14 positive monocytes or monocyte-derived macrophages that are treated by interferons. The cells may be used as such, or may be transfected with vectors encoding APOBEC3A or APOBEC3G to result in overexpression of these enzymes. The cells may be exposed to hypoxia and/or interferons. The interferons can be IFN gamma and IFN1.

In one embodiment, the effect of various compounds may be tested after the cells are exposed to conditions of hypoxia and/or interferons. For example, hypoxic conditions may be created in culture by exposing cells to 10% or less O₂ (with 5% CO2 and the rest nitrogen). In one embodiment, the O₂ is from 1 to 5%. In one embodiment, the O₂ is less than 1%. The cells may be exposed to the hypoxic conditions and/or interferons for desired lengths of time. For example, cells may be exposed for from 6 hours to 48 hours or more.

The interferons useful for the present methods include IFN gamma and IFN1. IFN1 is considered a ‘universal’ type I IFN . In one embodiment, the IFN1 comprises a hybrid of N-terminal IFNα-2 and C-terminal IFNα-1 produced in E. Coli. Useful range of interferon includes 50-500 U/ml for IFN gamma; 50 U/ml to 2,500 U/ml for IFN type 1 to induce APOBEC3A mediated RNA editing.

The agents identified by the methods of the present disclosure may be further tested for anti-tumor activity (such as those agents which inhibit C>U deamination) or for anti-viral activity (such as those agents which enhance C>U deamination activity).

The following examples are provided to further illustrate this invention.

### EXAMPLE 1

This examples demonstrates identification of APOBEC3A as an RNA editing enzyme.

Methods

Isolation and Culture of Cells

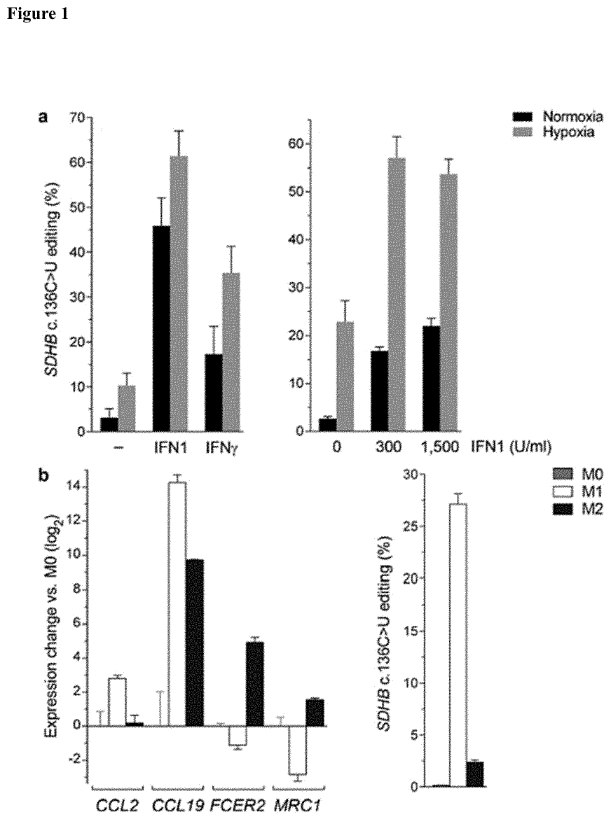

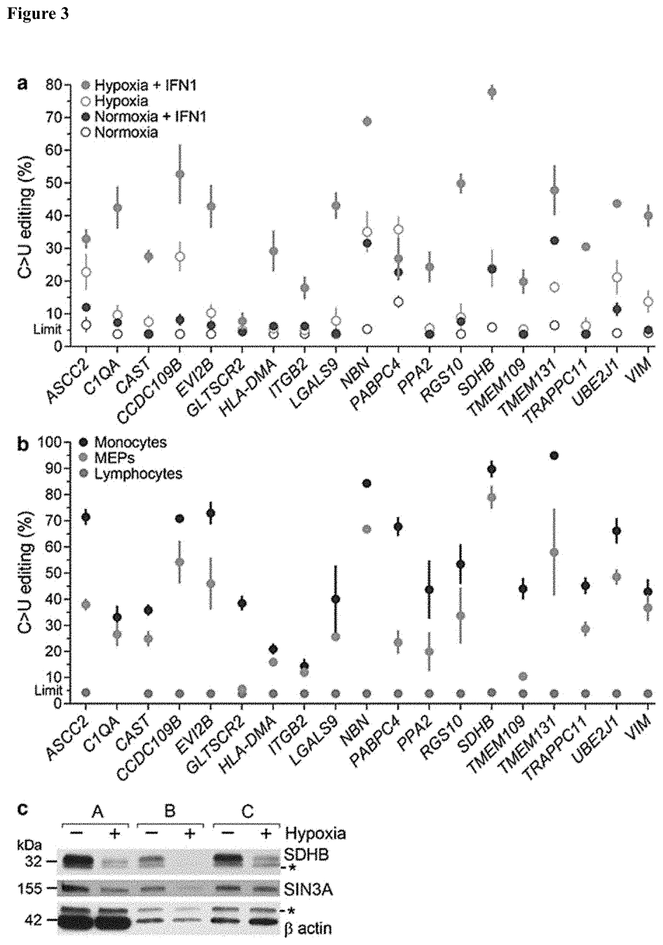

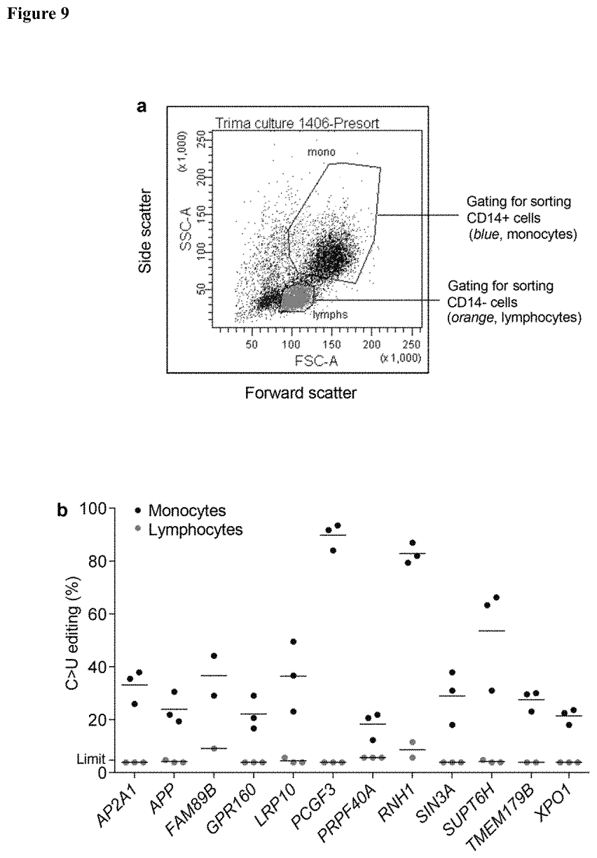

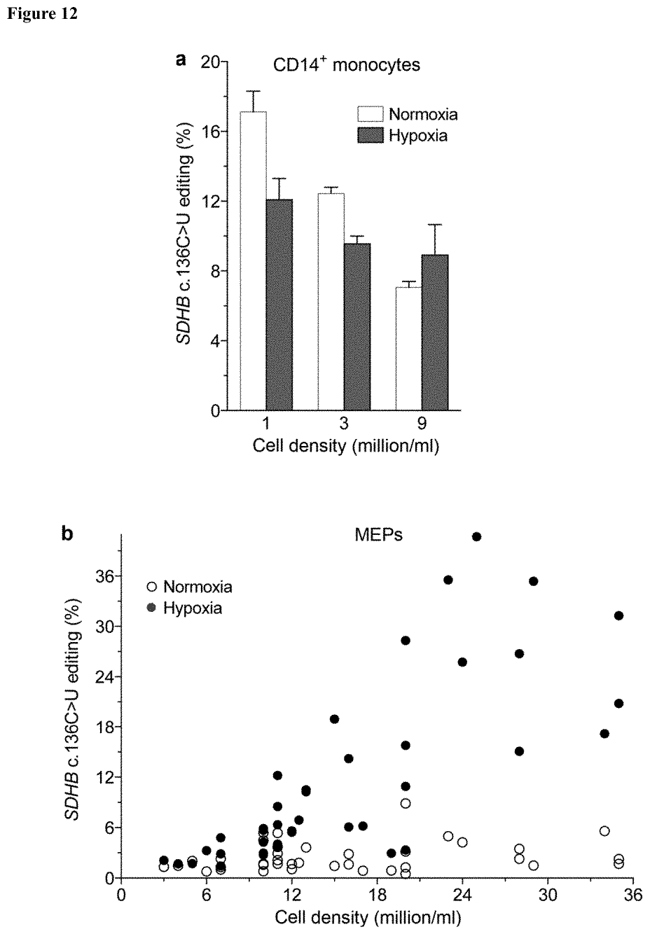

The TLA-HEK293T™ 293T human embryonic kidney cell-line was obtained from Open Biosystems® (Huntsville, Ala.). Peripheral blood mononuclear cells of anonymous platelet donors were isolated from peripheral blood in Trima Accel™ leukoreduction system chambers (Terumo BCT®, Lakewood, Colo.) after thrombocytapheresis, in accordance with a protocol approved by the institutional review board of Roswell Park Cancer Institute. A density gradient centrifugation method using polysucrose-containing Lymphocyte Separation Medium (Mediatech®, Manassas, Va.) was used for PBMC isolation. MEPs were prepared from PBMCs using the well-established cold aggregation method (Mentzer et a., Cell Immunol 101, 312-319 (1986) with slight modification. Briefly, PBMCs were subjected to gentle rocking at 4° C. for an hour and aggregated cells that sedimented through fetal bovine serum (FBS; VWR®, Radnor, Pa.) were collected as MEPs after 0.5-3 hours for high monocyte enrichment (˜70% monocytes as assessed by immunofluorocytometry for CD14), or after 8-16 hours for mild enrichment (˜20%-40% monocytes); the latter was used in all experiments except for the ones of FIG. 1a. MEPs were cultured at a density of 13-63 million per ml (mean=29 million per ml, n=16 experiments) in 1 or 2 ml per well of 6- or 12-well standard tissue culture plates under 5% CO2 in RPMI-1640 medium (Mediatech®) with 10% FBS, and 100 U per ml penicillin and 100 μg per ml streptomycin (Mediatech®). Monocytes and lymphocytes were isolated from MEPs based on light scattering and binding of a phycoerythrin-conjugated mouse anti-CD14 antibody (clone RM052, product number 6699509D, 1:40 dilution, Beckman Coulter®, Miami, Fla.) by flow cytometry on a FACS Aria™ II instrument with FACS Diva™ 6.0 software (BD Biosciences®, San Jose, Calif.) (FIG. 9a). CD14+ monocytes used in the experiment for FIG. 1b were isolated from PBMCs using mouse anti-CD14 antibody-conjugated microbeads and magnetic separation on an AutoMACS™ instrument (Miltenyi Biotec®. Auburn, Calif.). Monocytes used in the experiment for FIG. 3c were isolated from MEPs by immunomagnetic negative selection using EasySep™ Human Monocyte Enrichment Kit (Stemcell Technologies®, Vancouver, Canada). For the APOBEC3A knock-down experiment, monocytes of 70% CD14 positivity were isolated from PBMCs using a centrifugation-based method (Seager Danciger et al., J Immunol Methods 288, 123-134 (2004)) with a single-layer of iso-osmolar, 42.5% v/v solution of Percoll™ (GE Healthcare®, Pittsburgh, Pa.) in RPMI-1640 medium with 10% FBS. Except for the cells used in the experiment for FIG. 1b, all primary cells were used in experiments immediately after their isolation from PBMCs. Enhancement by hypoxia of SDHB c.136C>U RNA editing was not observed in cultures of previously cryopreserved CD14+ monocytes (FIG. 12a). Hypoxic induction of RNA editing was also not consistently observed in hypoxia if freshly isolated MEPs were cultured at a low cell density (<10 million cells per ml (FIG. 12b).

Generation and Polarization of Macrophages

CD14+ monocytes isolated from PBMCs by magnetic sorting and stored frozen in RPMI-1640 with 36% v/v FBS and 10% v/v dimethyl sulfoxide, were thawed and cultured for a week at a density of 0.25 million per ml with 50 ng per ml recombinant human macrophage colony stimulating factor (MCSF; Life Technologies®, Carlsbad, Calif.), 1× GlutaMAX™-I (Life Technologies®) and 1 mM sodium pyruvate (Mediatech®) to generate M0 macrophages. M0 macrophages were also similarly generated from fresh monocytes isolated from PBMCs by the Percoll™ -based method. For M1 or M2 macrophage polarization, M0 cells were treated for two days with 20 ng per ml recombinant human IFNy (Life Technologies®) and 100 ng per ml E. coli lipopolysaccharides (LPS; List Biological Laboratories®, Campbell, Calif.), or 20 ng per ml recombinant human interleukin 4 (Life Technologies®), respectively. RNA was isolated from cells using the Total RNA Purification Kit from Norgen Biotek® (Thorold, Canada).

Hypoxia and Interferon Treatments

For hypoxia, cells were cultured under 1% O2, 5% CO2 and 94% N2 in an Xvivo™ System (Biospherix®, Lacona, N.Y.). Human IFNγ and ‘universal’ type I IFN, a hybrid of N-terminal IFNα-2 and C-terminal IFNα-1, produced in E. coli were obtained from PBL Assay Science® (Piscataway, N.J.), and respectively used at 200 and 300-1,500 U per ml. Unless noted otherwise, hypoxia and/or interferon treatments were for 24 hours. Differential viability of MEPs after 1-day culture in normoxia versus hypoxia was not observed as evaluated by Trypan blue stain. Transfected 293T cells were subjected to hypoxia and/or interferon treatment 24 hours after transfection.

RNA Sequencing of MEPs

Indexed sequencing libraries were generated from RNA, isolated using TRIzol™ and without DNAse treatment, as per methods and reagents provided with the TruSeq™ Stranded Total RNA Sample Prep Kit with Ribo-Zero™ ribosomal RNA reduction chemistry (Illumina®, San Diego, Calif.). PCR for library generation employed 10 cycles. Electrophoresis of the libraries on Bioanalyzer™ 2100 instrument (Agilent®, Santa Clara, Calif.) showed highest peaks at 220-240 bp. Paired-end, multiplexed sequencing of libraries (three per flow cell lane) to generate reads of 101 bases (b) was performed on HiSeg™ 2000 instrument with TruSeq™ SBS and PE Cluster v3 Kits (Illumina®). CASAVA 1.8.2 software (Illumina®) was used for base-calling and de-multiplexing to obtain the raw RNA sequencing reads for further analyses. RNA sequencing of all six samples of this study was performed in one batch.

Macrophage RNA Sequencing Data of Beyer Et Al.

Paired-end, 101 b read sequence data generated using TruSeq™ RNA Sample Preparation Kit on Illumina® HiSeQ™ 2000 for paired M1 and M2 macrophages derived from CD14+ monocytes of three donors was obtained as SRA files from NCBI SRA (accession number SRP012015). Raw data in fastq format was extracted from the files with fastq-dump utility in NCBI SRA Toolkit 2.3.3 (ncbi.nlm.nih.gov/sites/books/NBK158900/).

Processing of RNA Sequencing Reads

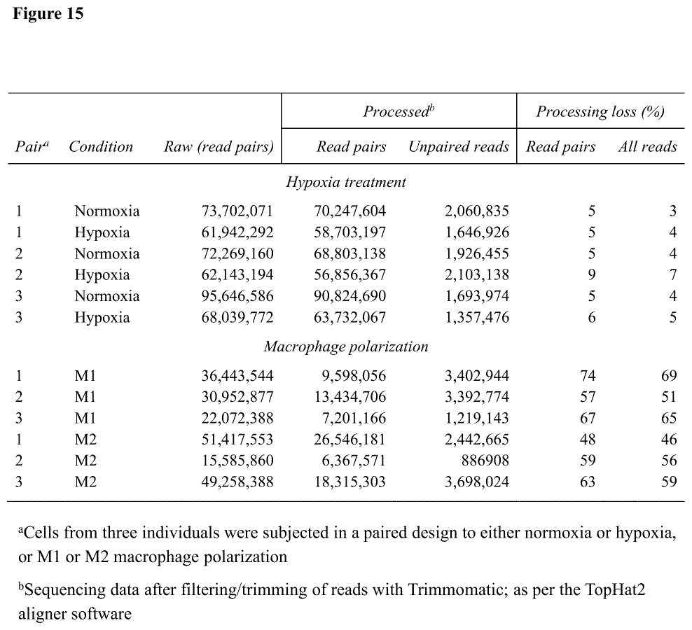

Quality of reads was assessed using FastQC 0.10.1 (URL: www.bioinformatics.babraham.ac.uk/projects/fastqc/). Trimmomatic 0.32 (URL: www.usadellab.org/cms/?page=trimmomatic) was used to trim 12 b from 5′ end, and remove adapter sequences and poor-quality bases from the reads. The Trimmomatic call was invoked with ‘HEADCROP:12 ILLUMINACLIP: TruSeq3-PE-2.fa:2:30:10:6:TRUE LEADING:5 TRAILING:5 SLIDINGWINDOW:4:15 MINLEN:30’, to satisfy these criteria, in order: (1) remove 12 b from the 5′ end of all reads because of base bias at these positions; (2) remove read segments that matched sequences of adapters and primers used for sequencing library preparation (the TruSeq3-PE-2.fa file provided with Trimmomatic was used); (3) remove leading/trailing bases with Phred33 base quality score <5; (4) using a sliding window of four bases, remove the most 5′ base if the average Phred33 base quality score of the four bases was <15; and, (5) completely discard trimmed reads with <30 remaining bases. Pair-mates of a fraction of raw reads were lost following this read processing with Trimmomatic. Processed read data thus had both paired and unpaired reads (FIG. 15).

Mapping of Processed RNA Sequencing Read Pairs

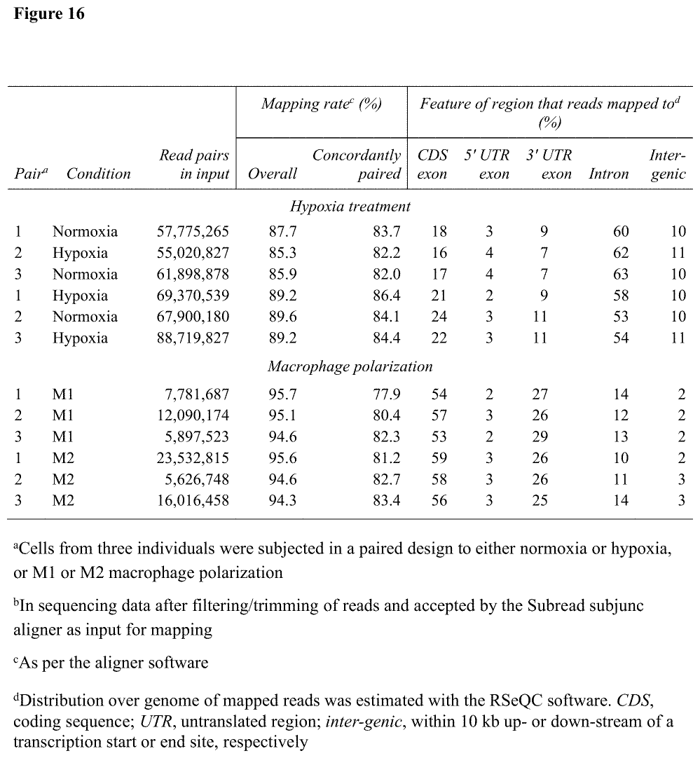

Processed read pairs were uniquely mapped to the hg19 genome with the Subread (Liao et al., Nucleic Acids Res 41, e108 (2013)) subjunc 1.4.3-pl aligner. The subread-buildindex command of Subread was used with default argument values to index the whole genome FASTA file for the UCSC hg19 genome assembly (obtained from Illumina® iGenomes). Subread subjunc command was used for mapping paired reads to the genome using the genome index with arguments u and H but otherwise default argument values to permit only unique mapping of a read and using Hamming distance to break ties when there were more than one best mappings. The nature of genomic regions that the reads mapped to was assessed using RSeQC 2.3.7. Mapping statistics are provided in FIG. 16. To obtain gene-level mapped read count data, the mapping results (BAM files) were analyzed with Subread featureCounts with reference to the GTF gene annotation file from UCSC (6 Mar. 2013 version) in the Illumina® iGenomes UCSC hg19 data with the following argument values specified: isStrandSpecific (s)=2 (0 in case of the macrophage data), GTF.featureType (t)=exon, GTF.attrType (g)=gene_id, isPairedEnd (p), allowMultiOverlap (O).

Processed RNA Sequencing Reads Mapping with TopHat2 Aligner

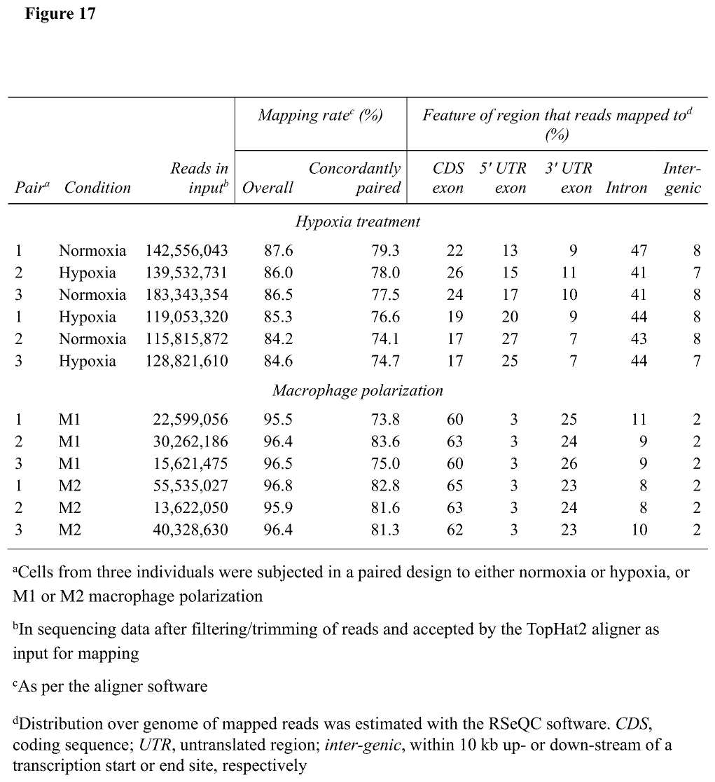

Processed reads, both paired and unpaired, were also mapped to the UCSC hg19 human genome assembly with the TopHat2 2.0.10 aligner, permitting only unique mapping of a read with up to three nucleotide mismatches. The bowtie2 index for the UCSC hg19 genome assembly was obtained from ftp.ccb.jhu.edu/pub/data/bowtie2_indexes, and the transcriptome index was built with TopHat2 using the GTF gene annotation file from UCSC (6 Mar. 2013 version) in the Illumina® iGenomes UCSC hg19 data. The tophat2 command was used with the transcriptome and bowtie2 genome indexes, and the following argument values specified: mate-inner-dist=−50, mate-std-dev=40, max-multihits=1, read-mismatches=3, read-edit-dist=3, no-novel-juncs (and library-type=fr-firststrand, in case of the RNA sequencing data of MEPs). The nature of genomic regions that the reads mapped to was assessed using RSeQC (URL: rseqc.sourceforge.net). Mapping statistics are provided in FIG. 17.

Generation of Mapped RNA Sequencing Read Pileups

After clipping overlaps of read pair-mates with clipOverlap utility in bamUtil 1.0.10 (URL: genome.sph.umich.edu/wiki/BamUtil), pileups were produced from the mapping data (BAM files) with mpileup in SAMtools 0.1.19 (URL: www.htslib.org), with computation of base alignment quality disabled (B), ‘anomalous’ reads permitted (A), maximum depth (d) set at 80,000, and aligner-reported read mapping quality (Q)>0 and Phred33 base quality score (q)>19 required.

Analyses of Mapped RNA Sequencing Read Pileups

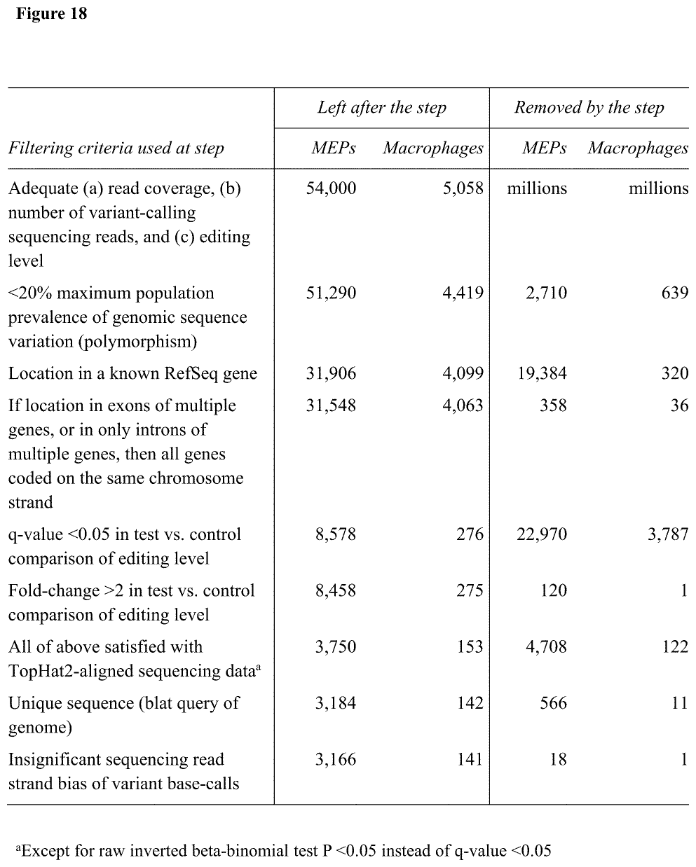

Paired comparison of pileups for the three pairs, from three different human donors, of normoxic and hypoxic MEP, or M2 and M1 macrophage samples was performed to identify genome sites with differential RNA editing in MEPs under hypoxia (test samples) compared to normoxia (control samples), or in M1 macrophages (test) compared to M2 macrophages (control). Python 2.7, R 3.0 and shell scripts were used for the analysis. Sites considered for analysis satisfied all of the following criteria regarding the A/T/G/C base-calling reads that covered them: (1) ≥20 calls (per sample, as for the other criteria here) in both samples of ≥1 pair, and ≥5 calls in all six samples; (2) ≥50% of calls for the reference human genome base in all test or all control samples; (3) ≥2 variant (other than the reference base) but identical base-calls in ≥2 test or ≥2 control samples, with ≥1 such calls in all test or all control samples, and ≤5 base-calls for a different variant in all six samples; and, (4) ≥95% reads with a base-call for either the reference or variant nucleotide in all six samples (thus, only one type of nucleotide change was considered for a site). Variation or editing level for sites was calculated as the ratio of variant base-calling- to the sum of variant and reference base-calling-read counts. Sites were then filtered by editing level, requiring: (1) ≥2.5% in ≥2 test or ≥2 control samples; (2) mean ≥5% for test or control samples; and, (3) range/mean ≥2 across all six samples (to reduce subsequent multiple testing). Variant sites with known maximum population prevalence >20% for identical sequence polymorphism (as per the popfreq_max ANNOVAR database, detailed below), or sites that did not map to a known RefSeq gene (URL: www.ncbi.nlm.nih.gov/refseq), or mapped to either exons of >1 RefSeq genes on both chromosome strands, or mapped to only introns of >1 RefSeq genes on both chromosome strands were excluded. Annotation data (BED files) for RefSeq gene introns and exons for the UCSC hg19 genome assembly were obtained on 21 Mar. 2014 using UCSC Genome Browser (URL: genome.ucsc.edu). The inverted beta-binomial (IBB) test for multiple paired count data was then applied to the remaining variant sites to identify sites that were differentially edited between the test and control samples. To control false discovery resulting from multiple testing, q-values were calculated from IBB test P values using the qvalue function in the qvalue Bioconductor package with these argument values specified: pi0.method=bootstrap, robust. Sites that were further considered had q-value <0.05 and >2-fold difference in either direction for editing level between test and control samples (fold-change values, capped at an absolute value of 104, were estimated by the IBB test) in analysis of Subread subjunc-aligned RNA sequencing data as well as an IBB test P value <0.05 and >2-fold difference in analysis of TopHat2-aligned RNA sequencing data. Sites were then filtered if either of their 5′ and 3′, 29 b-long, flanking genomic sequences, respectively with either the reference or variant base at the 3′ and 5′ end, aligned perfectly with the genome at another location; blat 35 (URL: genome.ucsc.edu) was used for this purpose. Finally, for filtering based on sequencing read strand bias, sites were filtered out if in the Subread subjunc-aligned data the variant base was called from a total across all six samples of >9 forward RNA sequencing reads but no reverse read, or vice versa, or if the number of forward and reverse reads were significantly different for either the test or control samples (IBB test P value <0.05). Numbers of sites that were left after and filtered by different steps of the analysis described here are noted in FIG. 18. RNA-level nucleotide change was deduced from DNA alteration based on the chromosome strand coding for the gene that a site mapped to, using the exon-bearing strand if a site mapped to both an intron and exon on opposite strands.

Analyses of RNA Editing Sites

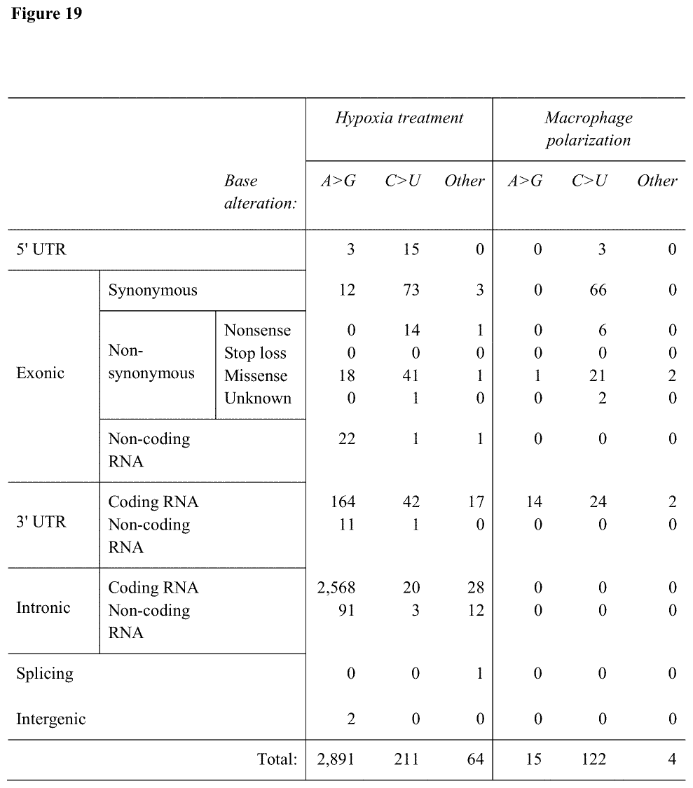

ANNOVAR tool (23 Aug. 2013 release;openbioinformatics.org/annovar) and ljb23_metalr (22 Feb. 2014), popfreq_max (21 Aug. 2013), RefSeq-based refGene (13 Nov. 2013), and dbSNP 138-based snp138 (13 Dec. 2013) ANNOVAR databases were used to annotate sites with information such as gene features they are located in, frequencies of known C/T genomic DNA polymorphism, and effects on amino acid coding. Coding genomic strand sequences flanking the editing sites were extracted from the whole genome FASTA file for the UCSC hg19 genome assembly (obtained from Illumina® iGenomes) with the getfasta utility in bedtools 2.17.0 (github.com/arq5×/bedtools), and these sequences were analyzed as transcript RNA sequences. Palindromic sequence context of editing sites was manually examined. RNA folding was predicted with ViennaRNA package 2.1.6 (tbi.univie.ac.at/RNA). These annotations are provided in FIG. 19. WebLogo 3 online tool was used to create sequence logos (weblogo.threeplusone.com). Gene set enrichment analyses for biological function, molecular process and PANTHER pathway ontologies were performed with PANTHER 9.0 (pantherdb.org/panther). Enrichment of a gene set with at least two genes for an ontology term, in comparison to the reference database for 21,804 genes, was assessed by binomial test and an FDR <5%, calculated from P values by the Benjamin-Hochberg method, was considered significant.

Gene Expression, RNA and Whole Exome Sequencing Data

Level 3, gene-level expression data determined by RNA sequencing with the UNC v2 pipeline were obtained from Broad Institute GDAC Firehose (2014_03_16 stddata run). RNA and whole sequencing data mapped to the hg19 genome assembly (BAM files) were obtained from Cancer Genomics Hub (University of California, Santa Cruz) respectively during February and March, and October 2014.

Analysis of TCGA Tumor RNA Sequencing Data for SDHB Editing

Pileups were generated as described above. Editing was deemed indeterminable for a sample if <99% of mapped reads had a base-call other than A or G, or there were <200 calls with none for A, or there were <100 calls with only one for A (SDHB gene is coded on the minus chromosome strand). Otherwise, C>U editing level was estimated as the ratio of G to the sum of A and G calls. Information on C/T single nucleotide polymorphisms in SDHB protein coding sequence was obtained from dbSNP (build 37).

RNA Sequencing Data Analysis for Varied Gene Expression

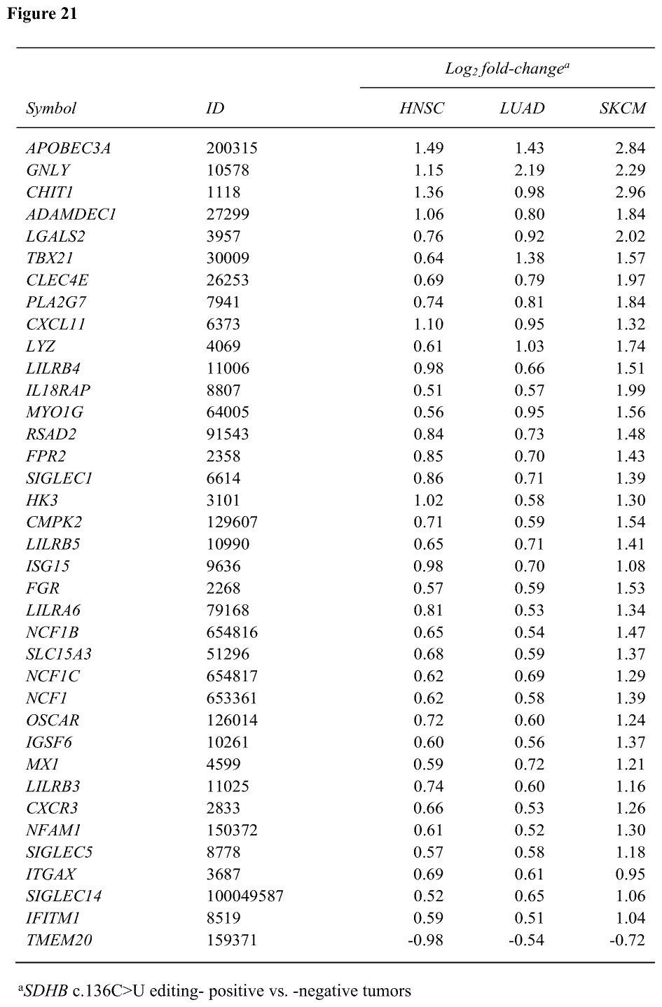

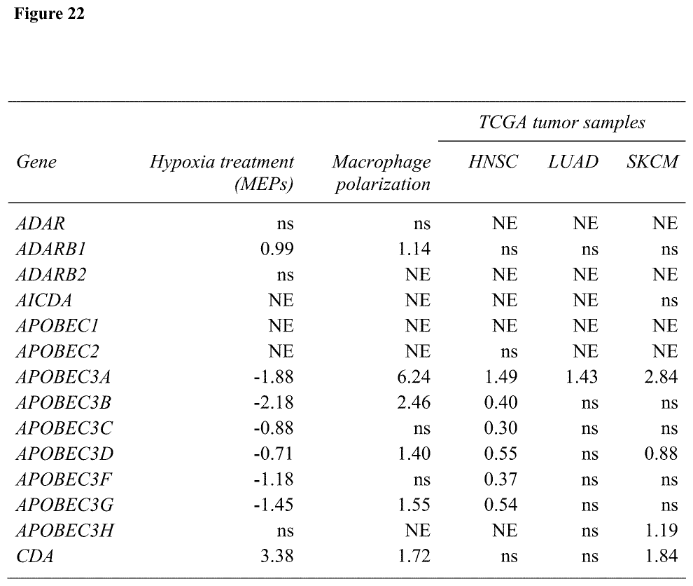

Gene-level raw count values of transcripts were analyzed with the edgeR Bioconductor package (version 3.2.3) for normalization with the trimmed mean of M-values method and inter-group comparison of gene expression by exact or likelihood ratio tests. For analyses of RNA sequencing data of tumor samples of TCGA, genes with raw count value >0 for ≥N samples, irrespective of group membership, where N equals the size of the SDHB c.136C>U editing-positive group, were considered as expressed, and values for prior.df and rowsum.filter parameters in estimateCommonDisp and estimateTagwiseDisp functions of edgeR were respectively set at 0.2 and 4N. An exact test was used to generate P values. For analyses of RNA sequencing data of MEPs and macrophages, genes with raw count value >1 for ≥3 samples, irrespective of group membership, were considered as expressed, and pair-wise comparison of gene expression between groups using generalized linear models with negative binomial distribution and a likelihood ratio test to generate P values was performed. False discovery rates (FDR) were estimated from P values with the Benjamini-Hochberg method, and genes with FDR <0.05 were considered as differentially expressed. Summarized results of differential gene expression analyses are provided in FIGS. 21 and 22.

Gene Expression Constructs and Site-Directed Mutagenesis

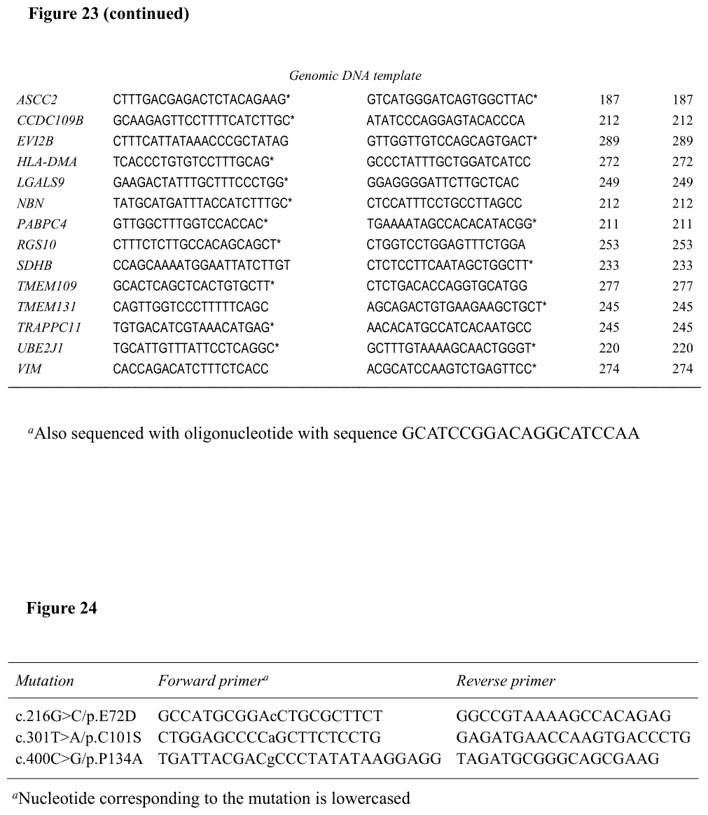

Sequence-verified plasmid constructs in pCMV6 vector for CMV promoter-driven expression of human APOBEC3A, APOBEC3G, CDA and SDHB cDNAs, with sequences matching NCBI RefSeq sequences NM_145699.2, NM_021822.1, NM_001785.1 and NM_003000.2, respectively, for the generation of C-terminal Myc-DDK-tagged APOBEC3A, and untagged APOBEC3G, CDA and SDHB proteins were obtained from OriGene® (Rockville, Md.; product numbers RC220995, SC122916, SC119015 and SC319204, respectively). An inducible bacterial expression construct for APOBEC3A with a C-terminal His6-tag in the pET21 vector was obtained from Dr. Jinwoo Ahn (University of Pittsburgh, USA). Site-directed mutagenesis of APOBEC3A constructs (c.216G>C/p.E72D, c.301T>A/p.C101S or c.400C>G/p.P134A; primer sequences shown in FIG. 24) was performed using Q5™ mutagenesis kit (New England Biolabs®, Ipswich, Mass.). Sequences of cDNA inserts of all of these constructs except that for SDHB were verified by Sanger sequencing. Insert-less pcDNA™ 3.1(+) vector (Life Technologies®) plasmid was used for control transfectants. The pRL-SV40 plasmid for SV40 promoter-driven expression of Renilla luciferase was obtained from Addgene® (Cambridge, Mass.). A LINE-1 plasmid (Mitra et al. Nucleic Acids Res 42, 1095-1110 (2014)) with an ˜6 kb human LINE-1 element bearing a CMV promoter-driven firefly luciferase cassette in its 3′ untranslated region was obtained from Dr. Judith Levin (National Institute of Child Health and Human Development, USA).

Transfection of Plasmid DNA

293T cells were transfected with plasmid DNA using the liposomal X-tremeGENE™ 9 DNA reagent (Roche®, Indianapolis, Ind.) or jetPRIME™ (Polyplus-transfection®, New York, N.Y.) reagents as per guidelines provided by the reagent manufacturer. Transfection efficiency with both reagents was 60%-80% as assessed by fluorescent microscopy of cells transfected with the pLemiR™ plasmid DNA (Open Biosystems®) for expression of a red fluorescent protein. Cells were harvested two days after transfection.

Knock-Down of APOBEC3A RNA in M1 Macrophages

A day before induction of M1 polarization, M0 macrophages at a density of 1 million cells per ml in 1 ml medium per well of 6-well plates were transfected with 100 nM of negative control (Silencer™ negative control no. 1, product number AM4611, Life Technologies®) or either or equimolar mix of two human APOBEC3A siRNAs (Silencer™ 45715 and 45810 respectively with sense sequences GACCUACCUGUGCUACGAATT (SEQ ID NO:1)and GCAGUAUGCUCCCGAUCAATT (SEQ ID NO:2), Life Technologies®) using Lipofectamine RNAiMAX™ (Life Technologies®) as per guidelines supplied by the manufacturer. IFNγ and LPS were added with 1 ml medium to each well to induce M1 polarization, and cells were harvested a day later.

LINE-1 Retrotransposition Assay

Briefly, firefly luciferase expression conditional to the retrotransposition of a human LINE-1 element from a plasmid DNA to the genome is measured in this assay. 293T cells at ˜50% confluence in 12-well tissue culture plates were co-transfected with 0.75 μg of the LINE-1 plasmid, 0.5 μg of pcDNA™ 3.1(+) or an APOBEC3A expression plasmid, 0.25 of pcDNA™ 3.1(+), and 1 ng of pRL-SV40 plasmid (per well). Transfectants were lysed after two days for measurement of their firefly and Renilla luciferase activities using Dual-Luciferase™ Reporter Assay System (Promega®). Retrotransposition was quantified as the ratio of firefly and Renilla luciferase activities.

Reverse Transcription and PCR

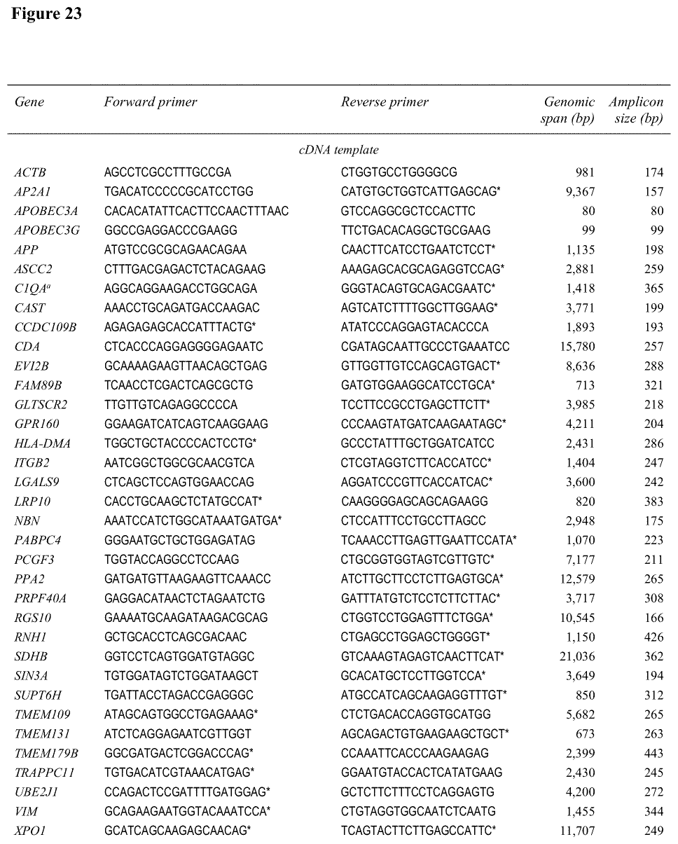

RNA was reverse transcribed with random DNA hexamers and/or oligo-dT primers using material and methods provided with the Transcriptor™ First Strand cDNA Synthesis (Roche®) or High Capacity cDNA Reverse Transcription (Life Technologies®) kits. PCR typically employed 35 cycles of amplification and an annealing temperature of 60° C. PCR oligonucleotide primers (Integrated DNA Technologies®, Coralville, Iowa) are listed in FIG. 23. Electrophoresis of PCR reactions on agarose gel was used to confirm the generation of a single product in a PCR. Primers used for PCR of cDNA templates were designed such that the amplicons spanned multiple exons. A blend of Taq and high-fidelity Deep VentR™ DNA polymerases (OneTaq™ , New England Biolabs, Ipswich, Mass.) was used in PCR to generate products for Sanger sequencing. For quantitative PCR to assess ACTB, APOBEC3A, APOBEC3B, CDA, SDHB, SIN3A and B2M gene expression, reactions using FastStart™ Taq DNA polymerase and SYBR™ Green I dye were performed on a LightCycler™ 480 System (Roche®). Quantification cycle (Cq) values were calculated by the instrument software using the maximum second derivative method, and the mean Cq value of duplicate or triplicate PCR reactions was used for analysis. TaqMan™ Gene Expression Assays from Life Technologies® with identification numbers Hs00234140_ml, Hs00171149_ml, Hs00233627_ml and Hs00267207_ml, or prepared in house were respectively used to quantify CCL2, CCL19, FCER2, MRC1 and ACTB with PCR performed on a 7900HT instrument (Life Technologies®) and Cq values determined with automatic baseline and threshold detection by SDS 2.4 software (Life Technologies®).

Sanger Sequencing

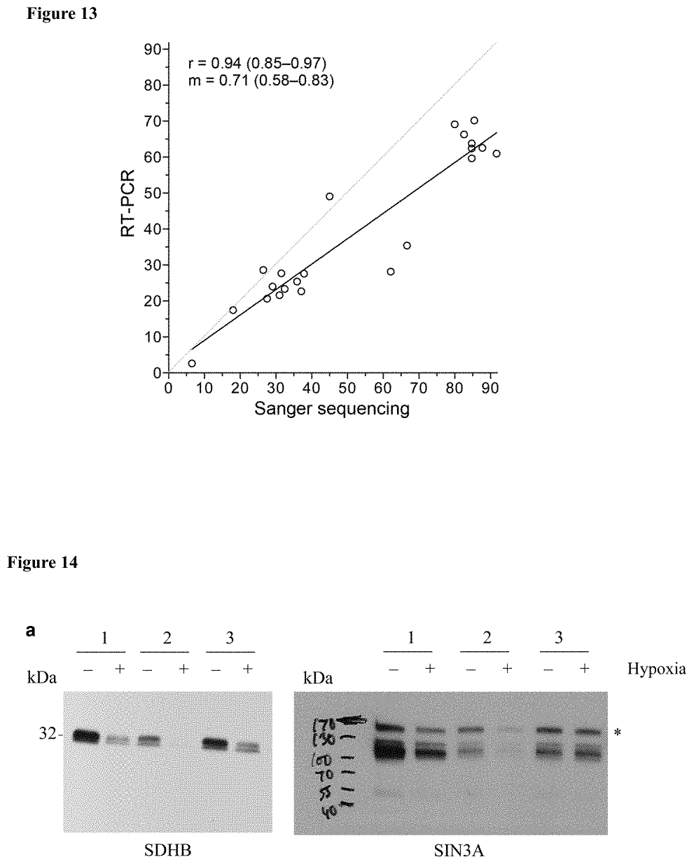

Sequencing primers (Integrated DNA Technologies®) are listed in FIG. 23. Candidate C>U RNA editing sites for which PCR-amplified genomic DNA and cDNA fragments were sequenced are noted in Table 1. PCR reactions were treated with ExoSAP-IT™ exonuclease (Affymetrix®, Santa Clara, Calif.) and then directly used for sequencing on 3130 xL Genetic Analyzer™ (Life Technologies®). Major and minor chromatogram peak heights at a nucleotide position of interest were quantified with Sequencher™ 5.0 software (Gene Codes®, Ann Arbor, Mich.) to calculate editing level for the position. Because the software identifies a minor peak only if its height is >5% of the major peak's, a relative minor peak height value of 4% was assumed to assign an editing level of 3.8% when a minor peak was absent. Appropriateness of this method to estimate RNA editing level was confirmed by comparing measurements of SDHB c.136C>U RNA editing level obtained with it against those obtained with allele-specific RT-PCR (FIG. 13).

Immunoblotting of Cell Lysates

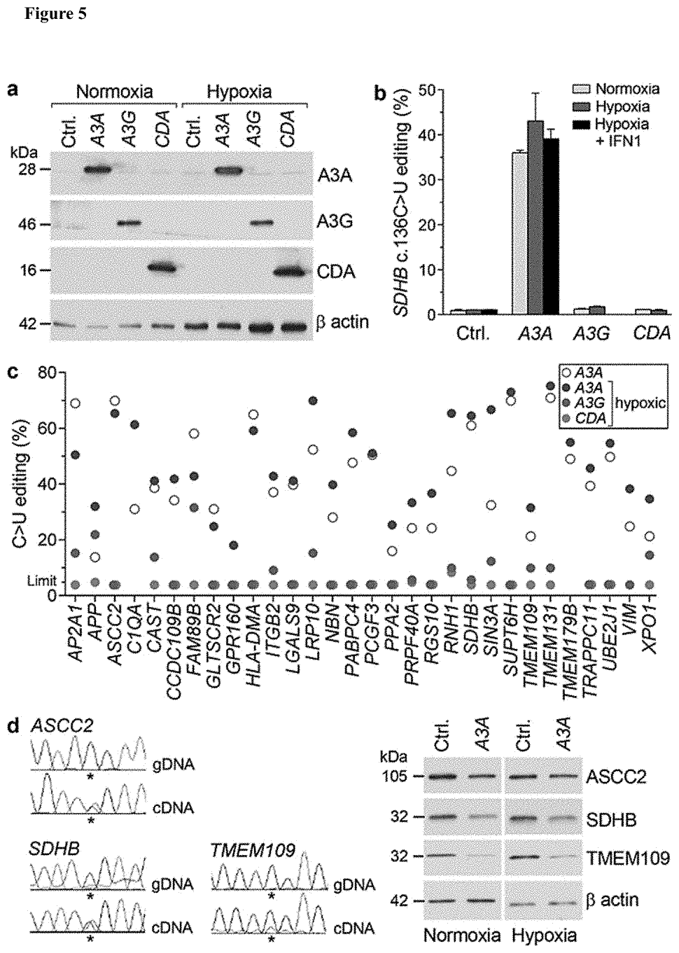

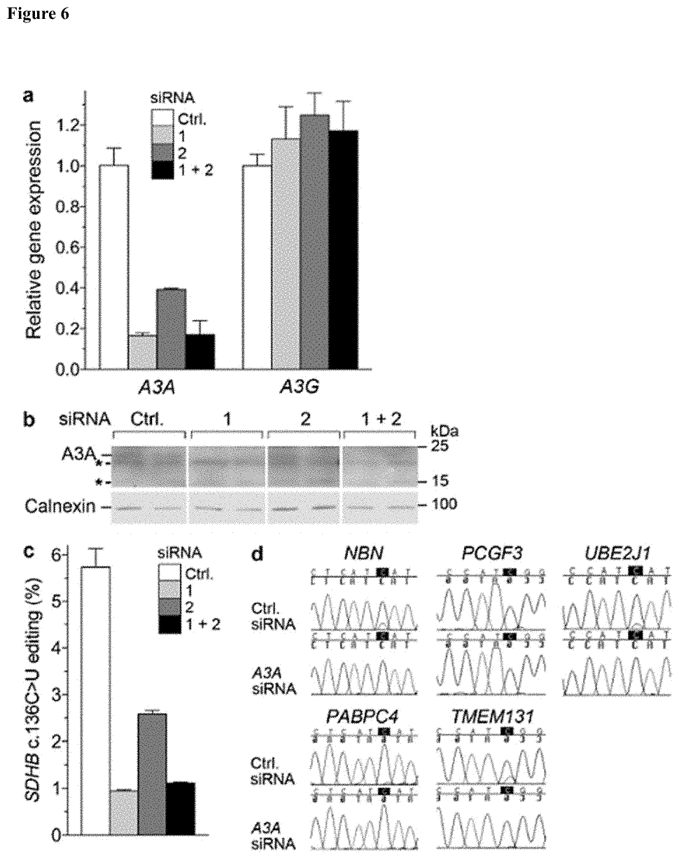

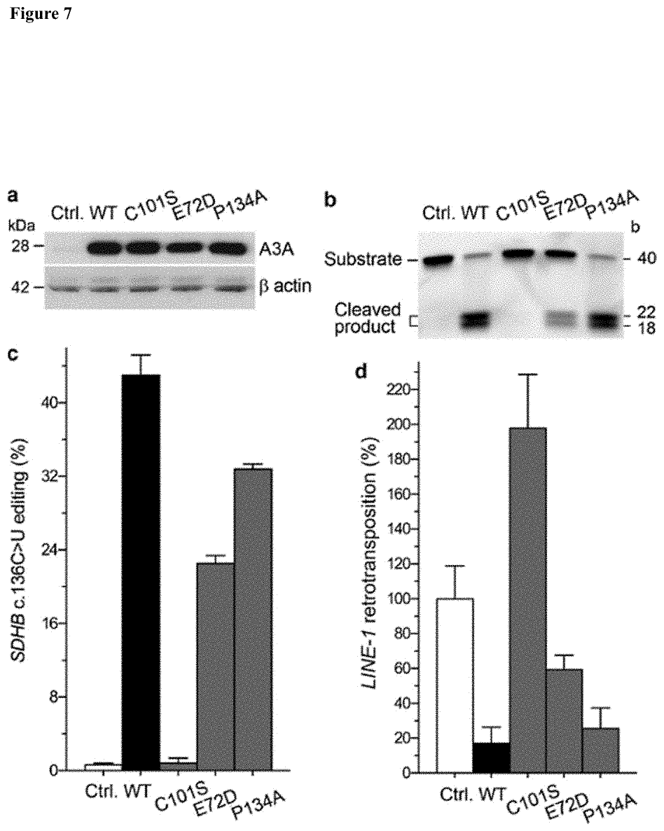

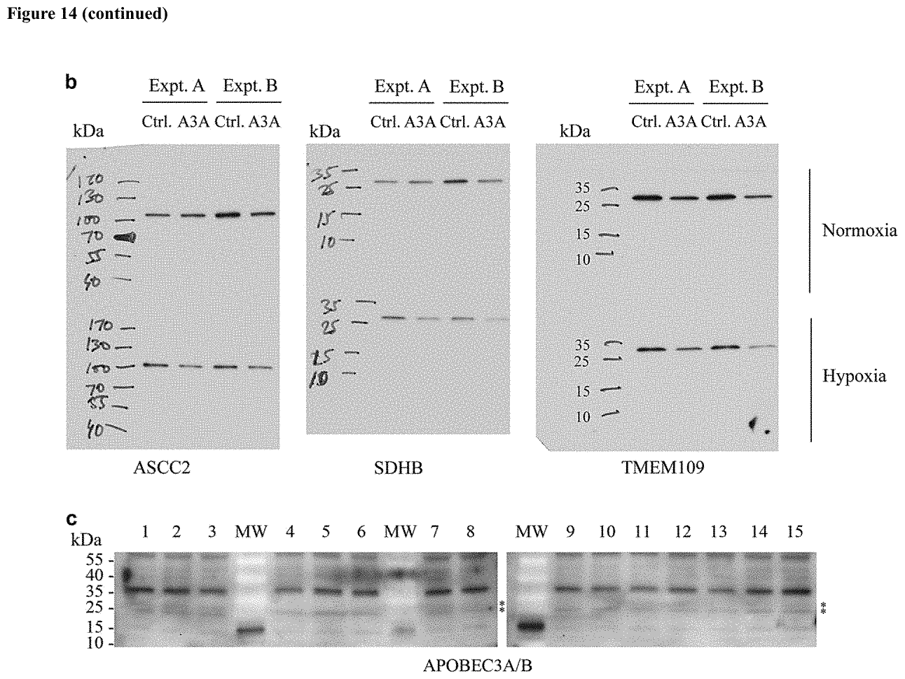

Whole cell lysates were prepared using M-PER™ reagent (Thermo Fisher®, Rockford, Ill.) with 1× Halt™ protease and phosphatase inhibitor cocktail (Thermo Fisher®). Reducing and denaturing polyacrylamide gel electrophoresis of 20 μg proteins in Laemmli buffer system was performed on pre-cast, 4%-15% gradient polyacrylamide gels (Mini-PROTEAN TGX™, Bio-Rad®, Hercules, Calif.). Proteins were then transferred to polyvinylidene difluoride membrane with a pore-size of 0.2 μm for 7 minutes at 1.3 A in a Bio-Rad® Trans-Blot Turbo™ apparatus. Membranes were incubated in Tris-buffered 0.15 M NaCl of pH 7.5 with 0.05% v/v TWEEN™ 20 (Sigma Aldrich®, Saint Louis, Mo.) and 5% w/v dried, non-fat, cow milk (Carnation™, Nestlé®, Glendale, Calif.) with antibodies at dilutions recommended by their manufacturers. Rabbit polyclonal anti-APOBEC3A (product number sc-130688, D-23, 1:200 dilution; used in the experiments for FIGS. 5a and 7a), anti-APOBEC3A/B (product number sc-292434, H-89; used in the experiment for FIG. 6b, 1:150 dilution), anti-ASCC2 (product number sc-86303, T-16; raised against peptide from internal region of human ASCC2, 1:200 dilution), and anti-TMEM109 (product number sc-133788, D-23; raised against human TMEM109 peptide of undisclosed sequence, 1:200 dilution) antibodies, and mouse monoclonal anti-CDA (product number sc-365292, D-5, 1:500 dilution) and anti-SDHB (product number sc-271548, G-10; raised against human protein of full length, 1:500 dilution) antibodies were obtained from Santa Cruz Biotechnology® (Santa Cruz, Calif.). Rabbit polyclonal anti-APOBEC3G (product number ab38604, 1:8000 dilution), mouse monoclonal anti-β-actin (product number AM4302, 1:15,000 dilution), and rabbit monoclonal anti-SIN3A (product number MABE607, EPR6780; raised against peptide near C-terminus of human SIN3A, 1:3000 dilution) antibodies were respectively obtained from Abeam® (Cambridge, Mass.), Life Technologies®, and EMD Millipore® (Billerica, Mass.). Rabbit polyclonal anti-calnexin antibodies (product number GTX10966, C3, 1:2000 dilution) were purchased from GeneTex (Irvine, Calif.). Horse radish peroxidase-conjugated, goat anti-mouse or -rabbit IgG antibodies were obtained from Life Technologies® and used at 1:2000 dilution. Luminata™ Forte Western HRP Substrate (EMD Millipore®, Billerica, Mass.) and CL-XPosure™ auto-radiography films (Thermo Fisher®) were used for chemiluminescent detection. Used membranes were stripped using a guanidine hydrochloride-based solution for re-probing with a different antibody. Uncropped scans of the immunoblots are shown in FIG. 14.

DNA Deamination Assay with Cell Lysates

The deamination assay described by Byeon et al. (Nat Commun 4, 1890 (2013)). was used. Whole cell lysates were prepared using M-PER™ reagent (Thermo Fisher®, Rockford, Ill.) with 1× Halt™ protease and phosphatase inhibitor cocktail (Thermo Fisher®). Briefly, 180 nM 5′ Alexa Fluor™ 488 fluorescent dye-labeled ssDNA substrate of 40 bases (Integrated DNA Technologies®) was incubated at 37° C. for an hour with 10 μl lysate and 10 units of E. coli uracil DNA glycosylase (New England Biolabs®) in 10 mM Tris (pH 8.0), 50 mM NaCl, 1 mM dithiothreitol (DTT) and 1 mM ethylene-diamine-tetraacetic acid (EDTA) in a volume of 50 μl. The reaction was stopped by adding 40 μg proteinase K (Life Technologies®) and incubating it for 20 minutes at 65° C. 10 μl of 1 N NaOH was added to the reaction which was then incubated at 37° C. for 15 minutes. After adding 10 μl of 1 N HCl, the reaction (10 μl) was electrophoresed on a 10% denaturing polyacrylamide gel. Typhoon™ 9400 Imager (GE Healthcare®) was used to scan the gel in fluorescence mode.

Purification of Recombinant APOBEC3A Proteins

Rosetta™ 2(DE3)pLysS E. coli (EMD Millipore®) transformed with a bacterial expression construct for C-His6-tagged APOBEC3A and grown in Luria broth at 37° C. were induced for expression of the recombinant protein with 0.3 mM isopropyl β-D-1-thiogalactopyranoside (IPTG) and cultured overnight at 18° C. Harvested cells were lysed with a French pressure cell (American Instrument Corporation®, Hartland, Wis.) and Ni-NTA His.Bind Resin™ (EMD Millipore®) was used as per manufacturer's instructions to purify APOBEC3A protein from the lysates by affinity chromatography. Isolated protein was concentrated using an Amicon™ Ultra-4 Centrifugal Filter Unit with Ultracel-3 membrane (EMD Millipore®; nominal molecular weight limit of 3 kDa). The concentrated protein was stored in 25 mM Tris (pH 8.0) with 50 mM NaCl, 1 mM DTT, 5% v/v glycerol, and 0.02% w/v sodium azide. Staining with Coomassie blue of protein preparation electrophoresed on a denaturing polyacrylamide gel indicated that it had APOBEC3A at >90% purity.

In Vitro SDHB Editing Assay

Whole cell lysates of 293T transfectants were prepared using lysis buffer containing 0.2% Surfact-Amps™ NP-40 (Thermo Fisher®), 30 mM 4-(2-hydroxyethyl)-1-piperazine-ethane-sulfonic acid (HEPES; pH 7.5), 100 mM KCl, 25 mM NaCl, 1.5 mM MgCl2, 1 mM DTT, and 0.5× Halt™ protease and phosphatase inhibitor cocktail, and stored with 10% v/v glycerol at −80° C. SDHB ORF RNA of ˜1.1 kb was generated by in vitro transcription of Xhol enzyme-linearized plasmid DNA using reagents and methods provided with the MEGAscript™ T7 Transcription Kit (Life Technologies®). SDHB RNA isolated from the transcription reaction was treated with DNAse I (Thermo Fisher®), and its integrity verified by electrophoresis on an agarose gel. For in vitro SDHB RNA editing assay, transfectant cell lysate (2-8 μl containing 21-84 μg protein) was incubated at 37° C. for 4-11 hours with 50 pg (125 amole) of SDHB RNA in a buffer containing 0.02 U per μl RNAse inhibitor (Protector™, Roche®), 100 mM KCl, 10 mM HEPES (pH 7.4), 1 mM DTT and 1 mM EDTA in a total volume of 50 μl. In vitro assays with purified APOBEC3A contained 5-10 μM APOBEC3A, 2-2.5 pM SDHB full-length RNA or single-stranded SDHB DNA (c.37-c.156), 10 mM Tris (pH 8.0), 50 mM KCl, and 10 or 100 nM ZnCl2 with or without 10 μM 1,10-phenanthroline (Sigma Aldrich®). The reactions were incubated for 2 hours at 37° C. RNA was purified from the reactions containing transfectant lysates or purified APOBEC3A using TRIzol™ (Life Technologies®) as per manufacturer's instructions. The c.136C>U editing of the exogenous RNA was assessed by allele-specific RT-PCR (Baysal et al., PeerJ 1, e152 (2013)) using a forward PCR primer (GGAATTCGGCACGAGGAC) (SEQ ID NO:3)that does not bind the cDNA of endogenous SDHB RNA. For Sanger sequencing to assess a 619 b segment of the RNA that spanned exons 1 to 5, the cDNA was amplified with primers with sequences GGTCCTCAGTGGATGTAGGC (SEQ ID NO:4) and TGGACTGCAGATACTGCTGCT (SEQ ID NO:5). For reactions with SDHB DNA as substrate, 4 μl of the reaction was directly used in PCR of volume 20 μl with primers with sequences TTGCCGGCCACAACCCTT (SEQ ID NO:6) and AGCCTTGTCTGGGTCCCATC (SEQ ID NO:7) to amplify the substrate for Sanger sequencing by the forward primer.

Other

SDHB gene expression and c.136C>U RNA editing was quantified by RT-PCR. Unless noted otherwise, total RNA, genomic DNA and plasmid DNA were isolated using material and methods provided with TRIzol™, DNA Wizard Genomic DNA Purification Kit (Promega®), and Plasmid Kit (Qiagen®, Germantown, Md.), respectively. RNA/DNA was quantified by spectrophotometry on a Nanodrop™ 2000 instrument (Thermo Fisher®). Proteins were quantified using Bio-Rad® Dc™ assay with bovine serum albumin standards. Statistical tests were two-tailed and were performed using R 3.0, Excel™ 2010 (Microsoft®, Redmond, Wash.), or Prism™ 6.0 (GraphPad®, San Diego, Calif.) software.

Results

SDHB RNA Editing in IFN-Treated MEPs and M1 Macrophages

Similar to hypoxia, an IFN-rich microenvironment is another factor that monocytes are exposed to during inflammation. IFNs also up-regulate expression of APOBEC3 cytidine deaminases, candidate enzymes that may be responsible for the SDHB c.136C>U RNA editing observed in monocytes. We therefore examined whether interferons induce SDHB c.136C>U RNA editing. As shown in the left panel of FIG. 1a, treatment of MEPs with type 1 interferon (IFN1; 600 U per ml) or IFNγ (200 U per ml) for 24 hours induced SDHB c.136C>U RNA editing in MEPs, both in normoxia and hypoxia under 1% O2 (Mann-Whitney U test P<0.01, comparing untreated and interferon-treated samples). The editing level in normoxic or hypoxic MEPs was increased ˜6-fold by IFN1 and ˜3-fold by IFNγ, suggesting that the induction of RNA editing with IFN1 was higher than with IFNγ (Wilcoxon rank sum test P<0.03, comparing samples regardless of hypoxia treatment). An additive effect of interferons and hypoxia on SDHB c.136C>U RNA editing was observed, and this was confirmed in an independent experiment in which matched MEPs of seven individuals were cultured under normoxia or hypoxia with 0, 300 or 1,500 U per ml IFN1 for 24 hours. Editing level in cells treated with both hypoxia and IFN1 was higher than in cells treated with only hypoxia or IFN1 (FIG. 1a, right panel; Wilcoxon test P<0.02, for both concentrations of IFN1).

IFNγ is an inducer of M1 (pro-inflammatory) polarization of macrophages, which are derived from monocyte precursors. We therefore examined and compared SDHB c.136C>U RNA editing in basal, unpolarized (M0), M1 and M2 macrophages. M0 cells were derived in vitro from CD14+ peripheral blood monocytes, and matched M1 and M2 macrophages were generated from the M0 cells by treatment with IFNγ and lipopolysaccharides, and interleukin-4, respectively. The SDHB RNA editing was found to be absent in M0 macrophages but occurred at an average level of ˜27% in M1 cells (FIG. 1b, right panel). The editing level was significantly lower in M2 macrophages (˜2%), suggesting a strong induction of editing in macrophages by M1 but not M2 polarization.

Wide-Spread RNA Editing in Hypoxic MEPs and M1 Macrophages

To investigate whether hypoxia affects editing of RNAs other than SDHB in MEPs, we performed RNA sequencing of matched normoxic and hypoxic MEPs of three healthy individuals. We also examined whole transcriptome RNA sequencing data obtained by Beyer et al. (PLoS One 7, e45466 (2012)) for matched M1 and M2 macrophages generated in vitro from peripheral blood monocytes of three individuals to determine whether M1 macrophage polarization differentially affects editing of other RNAs besides SDHB. Such comparison of whole transcriptomes of paired samples to identify RNA editing is less likely to falsely identify sequencing and mapping artifacts or genome sequence variations as RNA editing events.

About 84%-90% and 94%-97% of RNA sequencing reads of the MEPs and macrophages, respectively, could be uniquely mapped to the UCSC hg19 reference human genome (FIGS. 18-20). Calls made by the mapped reads for the reference base or a variation were counted along the genome, and paired count data were evaluated with the inverted beta-binomial test (Pham et al., Bioinformatics 28, i596-i602 (2012)) to identify genome positions at which the base variation level was differentially affected by hypoxia or M1 polarization with >2-fold change in either direction, with a q-value of <0.05 and a higher intra-group mean variation level of ≥5%. The type of RNA editing at a genome position was surmised from the base variation and the gene-coding chromosome strand at the position. The candidate RNA editing sites were filtered to remove likely false positives. Filtering criteria included identification of the site with a separate read-mapping software and location of the site within a known RefSeq gene (Methods and FIG. 18).

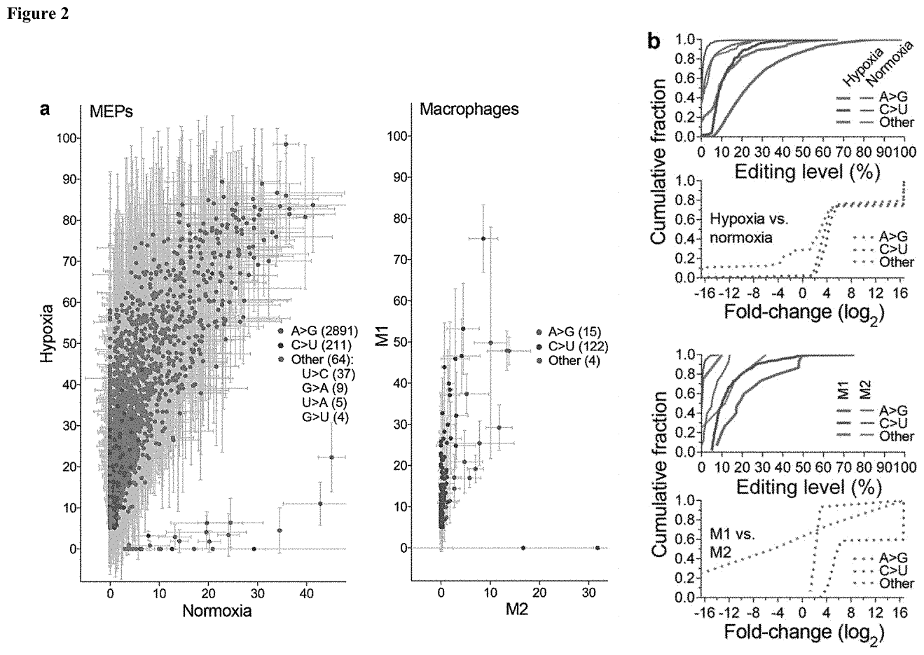

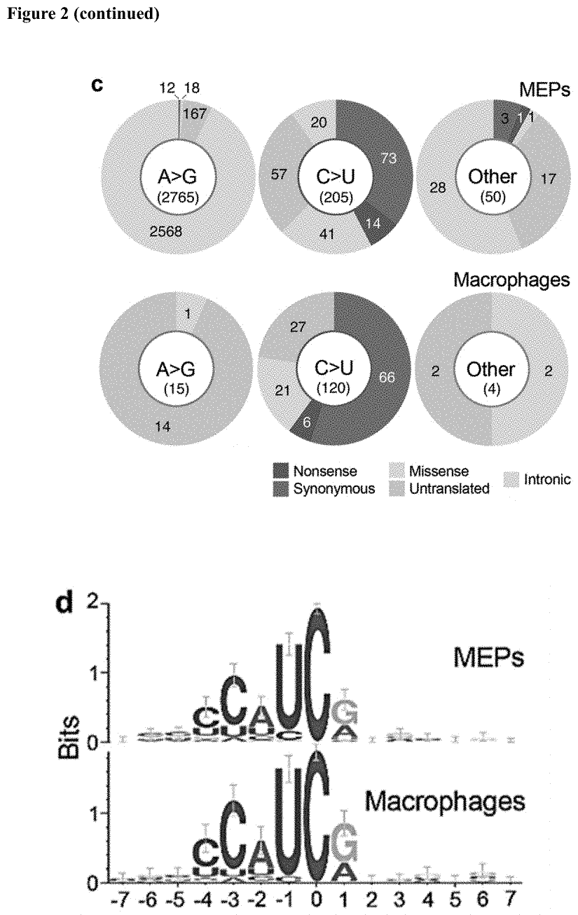

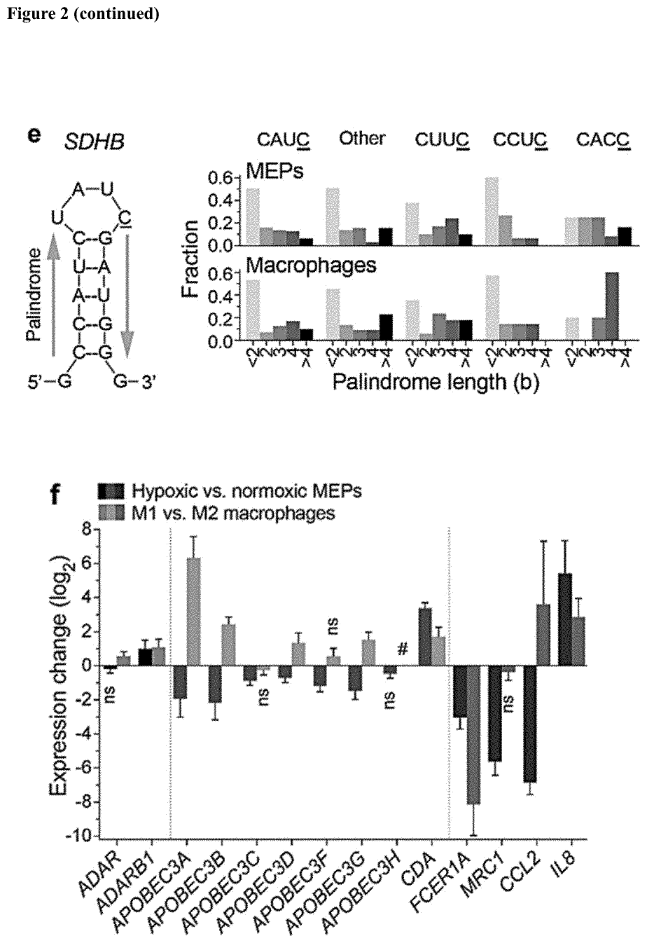

Putative RNA editing was found to be up- and down-regulated respectively at 3,137 and 29 sites by hypoxia in MEPs, and respectively at 139 and 2 sites by M1 compared to M2 macrophage polarization (FIGS. 2a and 2b, and FIG. 19). Editing in MEPs was of A>I (A>G) and C>U types at 91.3% and 6.6% of the sites, respectively, whereas these two types of editing respectively occurred at 10.6% and 86.5% of the sites in macrophages. A>G editing occurred at an overwhelming majority of the sites in MEPs, but only 1.0% of the A>G sites were in coding exons, causing 18 non-synonymous and 12 synonymous codon changes (FIG. 2c and FIG. 19). This is consistent with the known targeting of A>G RNA editing to non-coding sequences. On the other hand, 61.7% of the total 211 C>U sites (in 199 genes) were in coding exons, causing 55 non-synonymous and 73 synonymous codon changes. C>U editing accounted for 73.3% of all non-synonymous editing up-regulated by hypoxia in MEPs. In macrophages, 77.9% of the total 122 C>U sites (in 116 genes) were in coding exons, causing 27 non-synonymous and 66 synonymous codon changes.

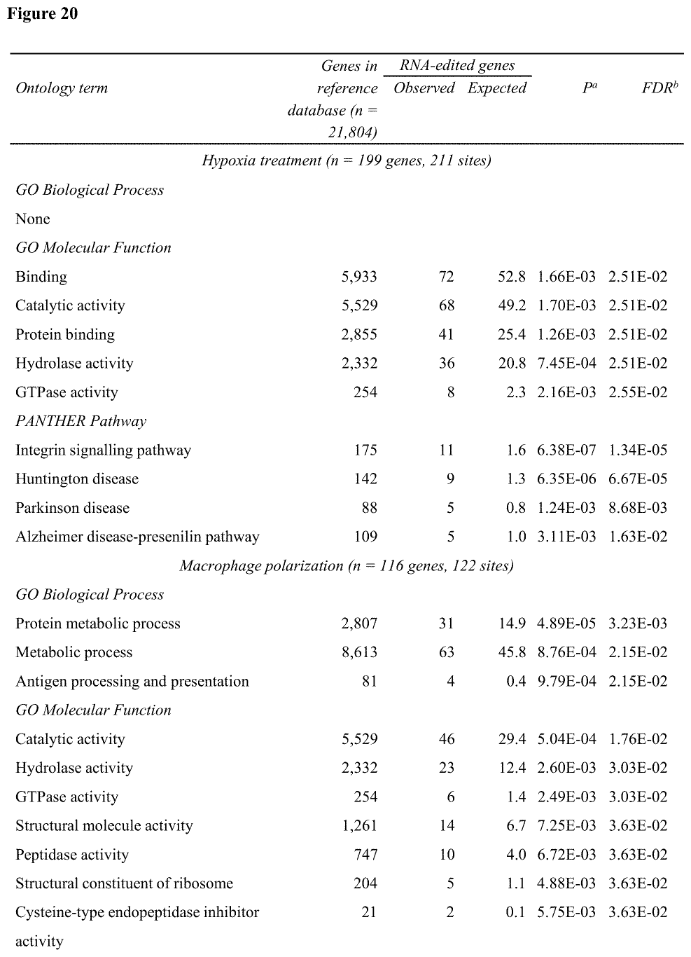

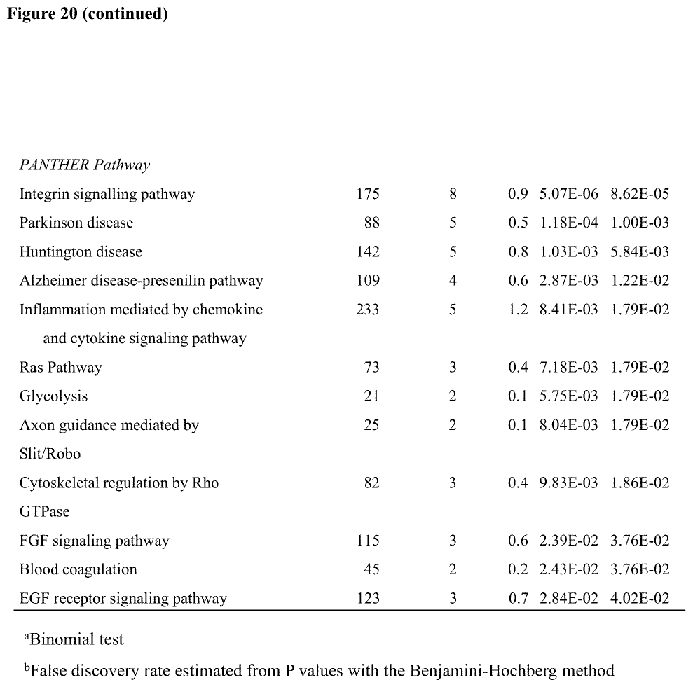

The average editing level in hypoxic MEPs was >10% and >20% for respectively 93 (45%) and 25 (12%) of the 206 C>U sites for which editing was up-regulated by hypoxia. In normoxic MEPs, the levels were <1% and <5% for respectively 162 (79%) and 202 (98%) of the 206 sites (FIG. 2b). Average C>U editing level in M1 macrophages was >10% and >20% for respectively 62 (51%) and 24 (20%) of the 122 sites. In contrast, levels in M2 cells were <1% and <5% for respectively 105 (86%) and 121 (99%) sites (FIG. 2b). Notably, 55 C>U RNA editing sites were shared by and upregulated in both the hypoxic MEPs and M1 macrophages. Editing of none of the 122 sites in M1 macrophages was down-regulated by hypoxia in MEPs. Ontology analysis of C>U RNA-edited genes of both MEPs and macrophages revealed enrichment for genes encoding for catalytic activities, and for genes in integrin-mediated signaling, and Alzheimer's, Huntington's and Parkinson's disease pathways (FIG. 20).

Sequence and Structural Contexts of C>U RNA Editing Sites

C>U editing sites were most commonly present within a CCAUCG sequence motif (edited site underlined), with CAUC and its CACC, CCUC, CUUC and UAUC 1-nucleotide (nt) variants present for approximately 79% and 85% of the editing sites of MEPs and macrophages, respectively (FIG. 2d and FIG. 19). Because the UAUC motif containing the SDHB c.136 nucleotide was flanked by palindromic sequences (FIG. 2e), we examined other C>U RNA editing sites to determine if the edited Cs in these were also flanked by palindromic sequences. About 51% and 52% of all edited NNNC sequences of MEPs and macrophages, respectively, were found to be flanked by short palindromic sequences of 2-7 nt (median=2 and 3 nt, respectively; FIG. 2e and FIG. 20). Examination of minimum free energy structures of 60 nt sequences bearing the edited C in middle showed that the C residue was present in the loop of a stem-loop structure for 72% and 67% of the sites of MEPs and macrophages, respectively (FIG. 19). These observations suggest that C>U RNA editing in MEPs and macrophages is catalyzed by cytidine deaminase(s) with particular target sequence and structure preference.

Validation of Site-Specific C>U RNA Editing in MEPs

Thirty-three non-synonymous C>U RNA editing sites (in 33 genes) that were identified in analysis of RNA sequencing data (FIG. 2a) were chosen for experimental validation of site-specific editing by Sanger sequencing of RT-PCR products. Eighteen of the 33 sites were identified in MEPs, three in macrophages, and 12 in both (Table 1). RNA editing for 31 of the 33 genes, including the three exclusively identified in macrophages, could be experimentally validated in MEPs (Table 1). The RNA editing level for 19 genes was quantified in MEPs of three donors. Editing for none of the genes was observed in normoxic MEPs, but was seen for all in MEPs treated with hypoxia with or without IFN1 (FIG. 3a). The additive effect of hypoxia and IFN1 on C>U RNA editing previously observed for SDHB (FIG. 1a) was also noticeable in the Sanger sequencing analyses for site-specific RNA editing of 18 other genes (FIG. 3a); the editing levels observed with combined hypoxia and IFN1 treatment (mean=38.2%) were significantly higher (Wilcoxon paired ranks test P<0.005) than the sum of those with IFN1 (mean=10.8%) or hypoxia (mean=14.5%) alone. Editing levels did not significantly differ between hypoxia and IFN1 treatments (ANOVA test P>0.05).

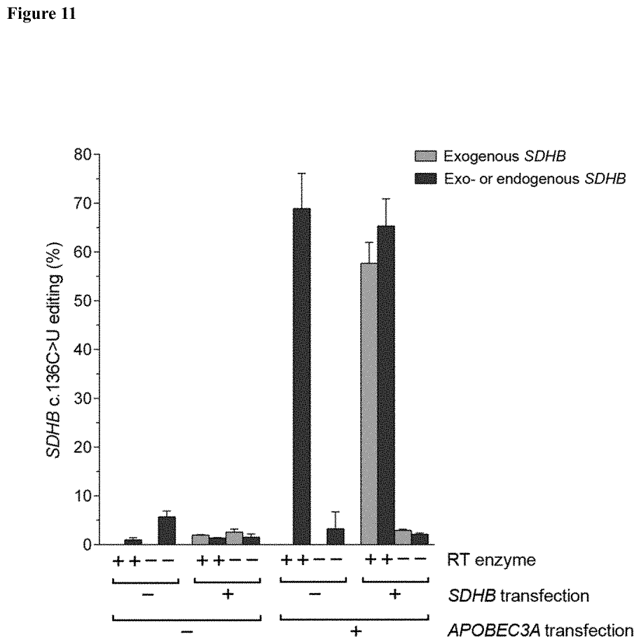

MEPs contain both monocytes and lymphocytes. To determine the RNA editing levels of the 31 experimentally validated genes in these individual cell types, Sanger sequencing of RT-PCR products of monocyte and lymphocyte isolates (FIG. 11a) of hypoxia- and IFN1-treated MEPs of another three individuals was performed (FIG. 3b, and FIG. 9b). Editing levels in monocytes were more than in their parent MEPs, and >20% for 29 genes and >80% for five (TMEM131, 95%; SDHB, 90%; PCGF3, 90%; NBN, 84%; RNH1, 83%). In lymphocytes, RNA editing was seen for only two of the 34 genes (FAM89B and RHN1, ˜8% level for each), suggesting that most of the differential C>U RNA editing in MEPs occurred in the monocytes.

For two of the transcripts for which the editing results in a nonsense codon change, SDHB (NCBI reference sequence NM_003000, exon 2:p.R46X) and SIN3A (NM_001145357, exon 20:p.Q1197X), the effect of hypoxia-induced C>U RNA editing on protein level was examined by immunoblotting assays of whole cell lysates of monocytes isolated from normoxic or hypoxic MEPs of three donors in a separate experiment. As shown in FIG. 3c, hypoxia treatment of MEPs resulted in a significant reduction of both SDHB (280 amino acid residues, NCBI reference sequence NP_002991) and SIN3A (1,273 residues, NP_001138829) in monocytes. Hypoxia also reduced SDHB and SIN3A RNA levels by an average of 4.7- and 1.6-fold in these three CD14+ monocyte samples, as tested by quantitative RT-PCR (normalized against the B2M gene). While this reduction could be at the transcriptional level, it could also be a result of post-transcriptional processes such as nonsense-mediated decay and microRNA targeting of the transcripts because of the sequence change resulting from their editing.

APOBEC3A Expression is Associated with SDHB RNA Editing

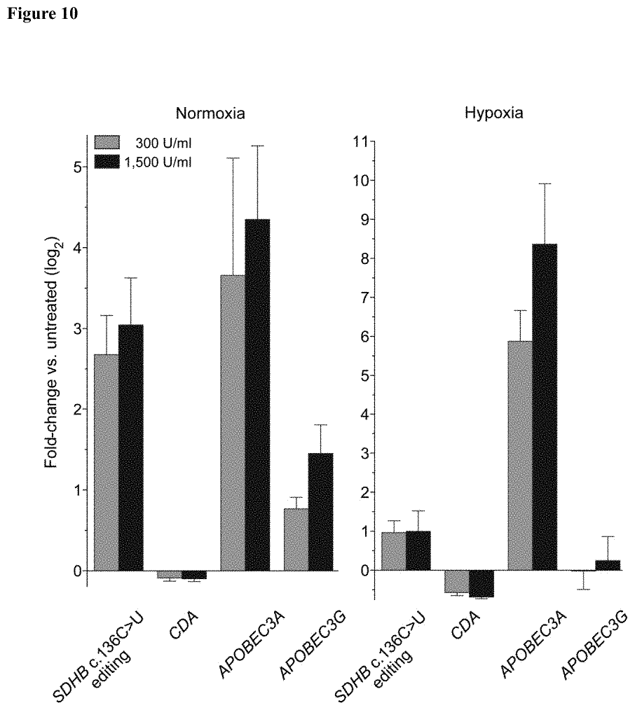

Next, we examined whether expression of any cytidine deaminase gene(s) was associated with SDHB c.136C>U RNA editing in monocytes and macrophages. CDA and the seven APOBEC3 genes were identified as expressed in RNA sequencing data of MEPs, and only CDA expression was up-regulated by hypoxia (FIG. 2f). Expression of APOBEC3A, the only APOBEC3 gene that is expressed at a higher level in monocytes compared to lymphocytes, was down-regulated by hypoxia. Expression of CDA and four APOBEC3 genes (A, B, D and G) was up-regulated in M1 compared to M2 macrophages, with APOBEC3A up-regulation being the highest (˜67-fold), whereas AID and APOBEC1, 2 and 3H were not expressed in macrophages (FIG. 2f). Up-regulation of APOBEC3A expression by IFN1 (also reported by Peng et al., J Exp Med 203, 41-46 (2006)), was seen in normoxic as well as hypoxic MEPs; IFN1 did not up-regulate CDA expression and up-regulated APOBEC3G expression only under normoxia (FIG. 10). Examination of changes in expression of cytidine deaminase genes in MEPs by hypoxia and IFN1, and in macrophages by M1 compared to M2 polarization, therefore suggested APOBEC3A and CDA as possible mediators of inducible C>U editing in MEPs and macrophages.

To further understand the association of cytidine deaminase gene expression with SDHB c.136C>U RNA editing, we evaluated RNA sequencing data in the Cancer Genome Atlas (TCGA) for three randomly chosen cancers, primary head and neck squamous cell carcinoma (HNSC), lung adenocarcinoma (LUAD), and secondary skin cutaneous melanoma (SKCM). Because tumors contain immune cells and can have hypoxic regions, we hypothesized that some degree of SDHB c.136C>U variation may be noticeable in the RNA sequences of the TCGA samples. Somatic SDHB c.136C>T mutation has not been identified in any TCGA sample for these cancers (data release 17 of International Cancer Genome Consortium (Hudson et al. Nature 464, 993-998 (2010)).

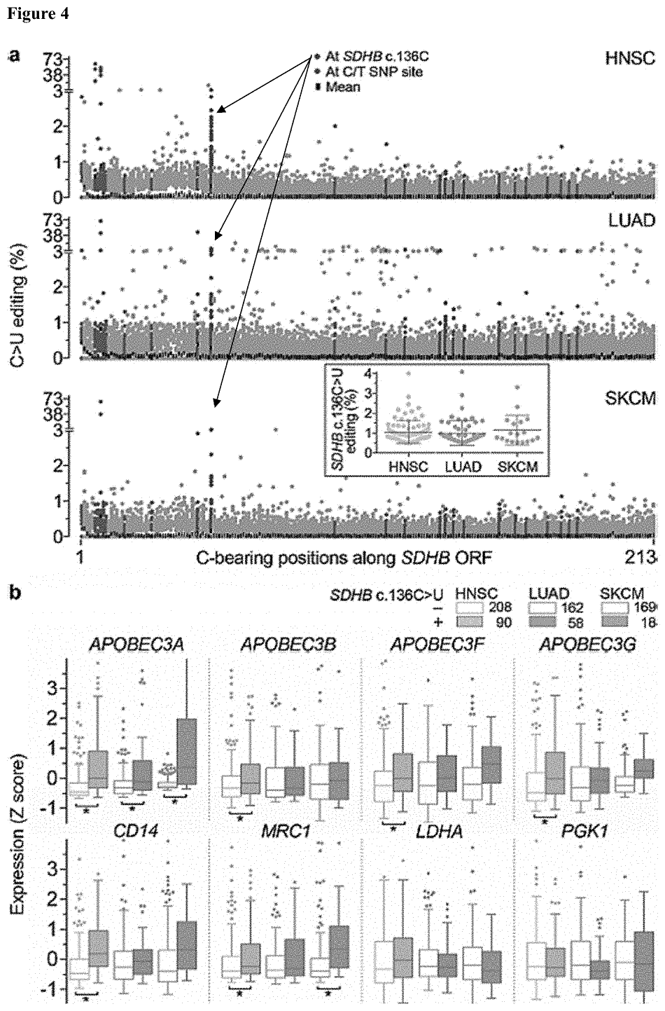

The scrutiny of TCGA's RNA sequencing data for the tumor tissues indicated putative C>U RNA editing of SDHB open reading frame (ORF) at c.136, but at no other site, in 30.2%, 26.4%, and 9.6% of the respectively 298 HNSC, 220 LUAD and 187 SKCM cases that were examined (FIG. 4a). The editing levels were low (˜1%), suggesting that it occurred only in a fraction of the cells of the tumors. Whole exome sequencing data for all eight tumors with editing level >2.25% showed complete absence of any sequence variation at c.136 at the genomic level (depth of coverage for SDHB c.136 ranging from 40-111, with mean=77). Comparison of gene expression between the editing-positive and -negative samples showed that APOBEC3A was the only cytidine deaminase gene whose expression was up-regulated in the editing-positive samples in all three cancers. Consistent differential expression of common hypoxia- or monocyte/macrophage-associated genes across all three cancers between editing-negative and -positive samples was not seen (FIG. 4b, and FIGS. 21 and 22).

APOBEC3A Overexpression Causes C>U RNA Editing in 293T Cells

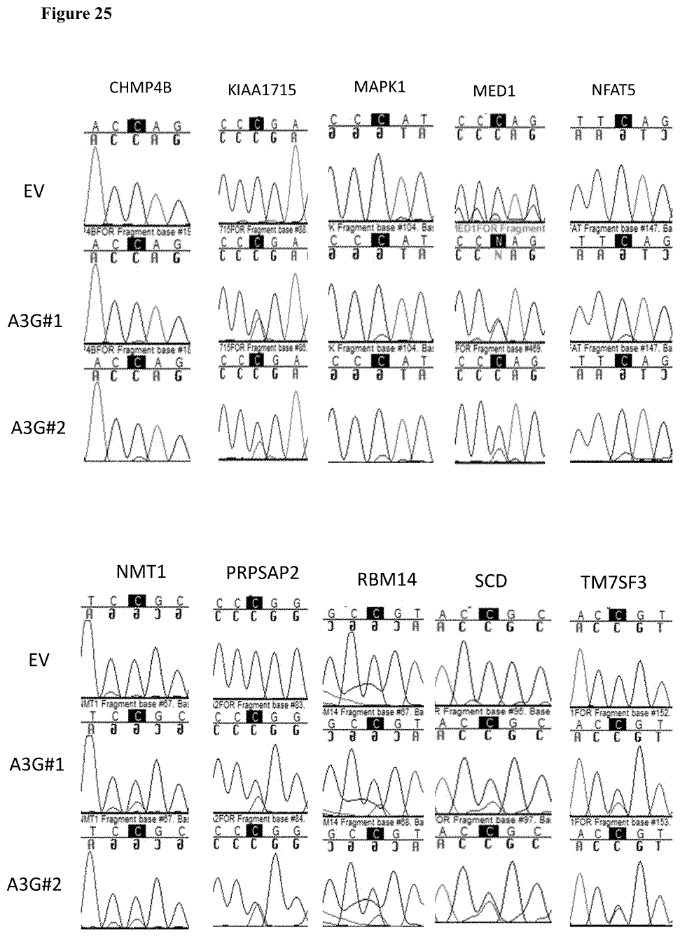

As noted above, the expression of APOBEC3A or CDA positively correlated the most with C>U RNA editing in cancer tissues, MEPs or macrophages (FIG. 25). To test if SDHB c.136C>U RNA editing can be induced by these two proteins, or by APOBEC3G whose expression is up-regulated by M1 macrophage polarization, their cDNAs were exogenously expressed in the human 293T embryonic kidney cell-line in which all three proteins were undetectable (FIG. 5a). Transient transfection of 293T cells for exogenous expression of APOBEC3A, but not APOBEC3G or CDA, induced SDHB c.136C>U RNA editing in the cells (FIG. 5b). Treatment of transfectants for 24 hours with hypoxia (1% 02) but not IFN1 (600 U per ml) mildly enhanced this editing (FIG. 5b). Previous studies have shown that intronic sequences are essential for A>I RNA editing, but not for APOBEC1-mediated C>U editing of APOB, which occurs in the nucleus after the APOB pre-mRNA has been spliced (Teng et al., Science 260, 1816-1819 (1993), Blanc et al., The Journal of biological chemistry 278, 1395-1398 (2003)). We found evidence for c.136C>U RNA editing of transcripts generated in vivo from a co-transfected, intron-less SDHB ORF cDNA expression construct in APOBEC3A transfectants, indicating that intronic sequences are not required for APOBEC3A-mediated RNA editing (FIG. 11).

Sanger sequencing of RT-PCR products of the 293T transfectants showed that exogenous APOBEC3A, but not CDA, also caused site-specific C>U RNA editing for 30 genes for which RNA editing was previously validated for MEPs (editing for EVI2B could not be examined because of low gene expression; FIG. 5c). This suggests that APOBEC3A mediates the transcriptome-wide C>U RNA editing that was noted for MEPs and macrophages (FIG. 2a). For most of the gene transcripts, hypoxia mildly increased the RNA editing levels, from an average level of 42% to 49% (Wilcoxon paired ranks test P=0.002; n=29). Sanger sequencing of PCR-amplified genomic DNA fragments of transfectants did not reveal C>T nucleotide variation at the editing site for any of the 23 genes that were examined (FIG. 5d). We tested the effect of APOBEC3A mediated RNA editing on the protein expression of 3 genes. Western blot assays of whole cell lysates of the transfectants for three proteins, ASCC2, SDHB and TMEM109, whose RNA transcripts were predicted to have p.R121X (in exon 4; NCBI reference sequence NM_032204, which encodes a protein of 757 aa), p.R46X (in exon 2; NM_003000, 280 aa) and p.R37X (in exon 2; NM_024092, 243aa) nonsense codon changes, respectively, because of RNA editing showed that exogenous APOBEC3A expression reduced levels of the proteins in 293T cells (FIG. 5d). In a separate RNA sequencing experiment, exogenous APOBEC3A expression in 293T cells was found not to affect SDHB RNA level in comparison to control transfectants, whereas it mildly but significantly affected ASCC2 and TMEM109 transcript levels, with fold-change values of ˜1.1 and 0.8, respectively. These results suggest that stop codons introduced by RNA editing may reduce wild type protein levels.

Notably, exogenous APOBEC3G also caused low-level, site-specific RNA editing for 11 genes in 293T transfectants (FIG. 6a); editing levels were highest for FAM89B and APP, for both of which the edited cytidine residue occurs in a CC sequence context that is known to be preferred by APOBEC3G for DNA deamination (Holtz et al., Nucleic acids research 41, 6139-6148 (2013)).

APOBEC3A Knock-Down Reduces RNA Editing in M1 Macrophages

To validate that APOBEC3A mediates SDHB c.136C>U RNA editing in M1 macrophages (FIG. 1b), we transfected M0 macrophages with small interfering RNA (siRNA) at 100 nM to knock down APOBEC3A RNA, induced their M1 polarization after a day, and examined the M1-polarized cells after another 24 hours. Transfection of cells with either of two different siRNAs predicted to target APOBEC3A, or their equimolar mix, led to a significant reduction in APOBEC3A transcript and APOBEC3A protein levels compared to cells transfected with a control siRNA that is not predicted to target APOBEC3A (FIGS. 6a and 6b). There was no effect on APOBEC3G RNA level in the cells, suggesting that the knock-down was gene-specific (FIGS. 6a and 6b). Reduction of APOBEC3A RNA level was associated with a significant reduction of SDHB c.136C>U RNA editing (FIG. 6c), indicating that APOBEC3A is a major determinant of this editing in M1 macrophages. Sanger sequencing of RT-PCR products was used to evaluate site-specific C>U editing level for transcripts of five other genes for which RNA editing in M1 macrophages had been noted in analysis of transcriptome sequencing data (FIG. 2a). Examination of the sequence chromatograms showed that macrophages transfected with an siRNA predicted to target APOBEC3A had a lower level of RNA editing for all five genes compared to cells that were transfected with the control siRNA (FIG. 6d).

RNA Editing by APOBEC3A Variants and Retrotransposition

The C101 residue of APOBEC3A is critical for binding of zinc, and the C101S APOBEC3A mutant completely lacks deamination activity against cytidines of ssDNA in vitro (Chen et al. Curr Biol 16, 480-485 (2006), Mitra et al. Nucleic Acids Res 42, 1095-1110 (2014)). As expected, cell lysates of the 293T transfectants exogenously expressing this mutant (FIG. 7a) did not cause deamination of the single cytidine residue of an ssDNA 40-mer (FIG. 7b). To test if C101 residue is essential for the observed RNA editing, we transfected 293T cells with the mutant cDNA. SDHB c.136C>U RNA editing, or site-specific C>U RNA editing for five other examined genes for which editing was observed in transfectants expressing the wild-type APOBEC3A was abolished in the C101S APOBEC3A transfectant (FIG. 7c). The E72D and P134A variants of APOBEC3A were previously shown to variably impair the ssDNA deamination activity of the wild-type enzyme (Mitra et al., Nucleic Acids Res 42, 1095-1110 (2014)). We found that whole cell lysate of 293T transfectant of E72D, but not P134A, was moderately impaired in the ssDNA deamination assay (FIGS. 7a and 7b). Unlike for C101S, the E72D variant was capable of C>U RNA editing of transcripts for SDHB and five other genes that were examined, though to lesser levels than the wild-type protein (FIG. 7c). The SDHB RNA editing level in transfectants of the P134A variant was ˜80% of that of transfectants expressing the wild-type APOBEC3A (FIG. 7c). These results suggest that the catalytic activity required for DNA deamination by APOBEC3A is also important for RNA editing.

APOBEC3A suppresses retrotransposition in cell-based assays and this suppression is dependent on its ssDNA cytidine deaminating catalytic integrity (see discussion). To test whether RNA editing and retrotransposition suppressing functions of APOBEC3A are linked, we tested the effect of mutations on LINE1 (L1)-retrotransposition. We found that the ability of the E72D, C101S and P134A variants to inhibit retrotransposition paralleled their RNA editing activities (FIG. 7d). These findings indicate that mutations in E72, C101 and P134 residues of APOBEC3A affect the protein's ssDNA and RNA deamination, and anti-L1 retrotransposition activities in a similar manner.

In Vitro Deamination of SDHB RNA and ssDNA by APOBEC3A

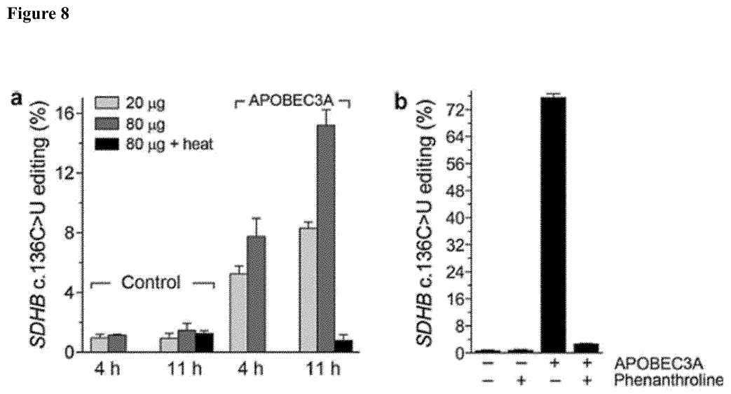

The various observations thus far noted suggest that APOBEC3A can deaminate cytidines in RNA. To demonstrate that APOBEC3A can edit c.136C>U in SDHB RNA in vitro, an SDHB ORF RNA of ˜1.1 kb with an artificial sequence at its 5′ end was incubated with whole cell lysates of 293T transfectants. Editing of the RNA at c.136 was quantified by allele-specific RT-PCR with a 5′ primer that was specific to the artificial sequence and using the same 3′-primers as described (Baysal et al., PeerJ 1, e152 (2013)). Lysate expressing APOBEC3A but not a control transfectant induced C>U editing of the exogenous SDHB RNA at c.136 in a time- and dose-dependent manner, and this activity was not seen with the heat-inactivated lysate (FIG. 8a). To further validate the RNA editing activity of APOBEC3A, in vitro editing assays were performed with purified APOBEC3A. Incubation of in vitro transcribed SDHB RNA with His6-tagged APOBEC3A protein showed site-specific editing of the SDHB RNA in vitro (FIG. 8b). Chelation of zinc in the deamination reaction with 1,10-phenanthroline abolished the editing (FIG. 8b). An ssDNA of 120 bases containing the SDHB cDNA sequence (c.37-c.156) too was deaminated at c.136 by the recombinant APOBEC3A protein. Thus, whereas cytidines of both SDHB ssDNA and RNA can be deaminated in vitro by APOBEC3A, deamination sites of RNA appear to be highly selective which may reflect a requirement for a more complex sequence or structure context.

Discussion

In this study, we demonstrate that APOBEC3A, a cytidine deaminase highly expressed in myeloid cells, is a C>U RNA editing enzyme that modifies the monocyte/macrophage transcriptome. The RNA editing in monocytes is activated by hypoxia and interferons in both independent and additive manners (FIGS. 1a and 2a), and in monocyte-derived macrophages by M1 but not M2 polarization (FIGS. 1b and 2a). These findings represent the discovery of the first mammalian RNA-editing cytidine deaminase enzyme since the identification of APOBEC1 in 1993, unveil a previously unrecognized function for the APOBEC3 family of genes, markedly expand our knowledge of C>U RNA editing events, and highlight a significant effect of micro-environmental factors on such editing.

The RNA editing activity of APOBEC3A (FIG. 8b) provides a new perspective to understand the anti-viral and -retrotransposition functions of APOBEC3A and possibly other APOBEC3 genes. APOBEC3A has been shown to strongly inhibit retrotransposons and diverse viruses including parvoviruses, alpharetroviruses, HTLV-1 and HIV-1 in the early stages of infection in myeloid cells (Ooms, et al., Journal of virology 86, 6097-6108 (2012), Arias et al., Front Microbiol 3, 275 (2012), Wiegand et al., Journal of virology 81, 13694-13699 (2007), Berger et al., PLoS pathogens 7, e1002221 (2011)). The mechanism by which APOBEC3A inhibits these agents is poorly understood. We find that the RNA editing and anti-LINE-1 retrotransposition abilities of APOBEC3A are similarly affected by E72D, C101S and P134A mutations (FIGS. 7c and 7d). This is consistent with the possibility that the newly discovered RNA editing activity of the host RNAs by APOBEC3A may provide a DNA deamination-independent mechanism for the inhibition of viruses and retrotransposons by the protein. The association established in this study between up-regulation of APOBEC3A-mediated C>U RNA editing of cellular transcripts and hypoxia or interferon-treatment of monocytes and M1 polarization of macrophages (FIGS. 1 and 2a) also supports this notion.

Non-synonymously C>U RNA-edited genes identified in this study may represent players that mediate the anti-viral and -retrotransposition function of APOBEC3A.

APOBEC3A is believed to deaminate foreign but not host genomic DNA in primary cells, and previous studies have demonstrated the deamination activity of the enzyme against ssDNA but not RNA (Mitra et al., Nucleic Acids Res 42, 1095-1110 (2014), Stenglein et al., Nat Struct Mol Biol 17, 222-229 (2010). Our data (FIG. 2d) indicates that the enzyme deaminates cytidines of RNA within CAUC or its 1-nt. variant motifs that are flanked by palindromic sequences. It thus appears that previous studies failed to observe the RNA editing activity of APOBEC3A, which is known to bind RNA, in part because they used substrate RNAs containing a non-specific sequence.

An important finding of this study is that hypoxia independently activates C>U RNA editing to levels comparable to those induced by IFN1 (FIGS. 1a and 3a). Moreover, stimulation of MEPs by hypoxia and IFN1 together additively increases editing, with levels reaching over 80% for five of the 31 genes validated by Sanger sequencing (FIG. 3a). Since hypoxia is pervasive in inflamed tissue, this suggests that RNA editing has the potential to substantially alter certain cellular proteins in virus-infected cells in vivo. How hypoxia activates C>U RNA editing is currently unknown. Although up-regulation of APOBEC3A expression may underlie the activation of C>U RNA editing by interferons (FIGS. 1a and 3a), APOBEC3A expression in MEPs is down-regulated by hypoxia (FIG. 2f). Hypoxic stimulation of C>U RNA editing in these cells may therefore be caused by an alternative mechanism such as enhanced translocation of the enzyme to nucleus, where A>I and APOBEC1-mediated C>U RNA editing are known to occur. Monocytes routinely encounter hypoxia upon their exit from the highly oxygenated bloodstream to inflamed tissues, but the oxygen-sensing mechanisms in these cells are poorly understood. We find that RNAs encoding for both the SDHA and SDHB subunits of mitochondrial complex II are targets of hypoxia-induced C>U editing (FIG. 19), suggesting that suppression of this complex facilitates hypoxia adaptation in pro-inflammatory monocytes and macrophages.

Monocytes and monocyte-derived pro-inflammatory macrophages play an important role in pathogenesis of common diseases including infectious diseases, obesity, cancer, Alzheimer's disease and atherosclerosis. We found that APOBEC3A causes non-synonymous RNA editing of transcripts of the APP, AP2A1, CAST, LRP10 and XPO1 genes (FIG. 5c) that are implicated in pathogenesis of Alzheimer's disease through regulation of amyloid precursor protein. Analyses of RNA sequencing data of this study shows that up-regulation of CD33 gene expression, which is associated with AD susceptibility, also occurs in MEPs under hypoxia (3.1-fold, FDR=0.0002, Fisher's exact test), and in M1 relative to M2 macrophages (2-fold, FDR=0.012, Fisher's exact test). It is thus possible that inflammation and hypoxia create multiple risk factors for chronic diseases like Alzheimer's disease through RNA editing and altered gene expression in monocytes/macrophages.

Our findings reveal an unprecedented extent and level of protein-recoding RNA editing in innate immune cells in response to certain micro-environmental factors associated with inflammation, which is mediated by APOBEC3A. In the light of important role which APOBEC3A plays in restricting diverse viruses and retrotransposons, these findings suggest a deaminase-dependent cellular RNA editing model which can be used to investigate the molecular bases of these restrictions and to identify agents that affect RNA editing.

RNA Sequencing data of MEPs were deposited in NCBI Sequence Read Archive (SRA) with accession number SRP040806.

TABLE 1
Candidate sites experimentally examined for validation of C > U RNA editing^{a}

| | | | Editing level (%)^{d} | | | |
|---|---|---|---|---|---|---|
| | Chromosomal | cDNA and amino | MEPs | | Macrophages | |
| Gene | position^{b} | acid change^{c} | Normoxia | Hypoxia | M2 | M1 |
| AP2A1 | 19:50295238 | C520T, R174X | 0 | 15.5 | NA | NA |
| APP | 21:27326988 | C1546T, R516C | 0 | 8.5 | NA | NA |
| ASCC2 | 22:30221126 | C202T, R68X | 0.9 | 19.1 | NA | NA |
| C1QA | 1:22965523 | C361T, R121W | NA | NA | 0.1 | 10.7 |
| CAST | 5:96106257 | C1826T, S609F | 0 | 7.2 | 0.1 | 5.2 |
| CCDC109B | 4:110605624 | C638T, S213L | 0.3 | 20.4 | NA | NA |
| EVI2B | 17:29632509 | C119T, S40L | 0 | 18.1 | 0.6 | 12.1 |
| FAM89B | 11:65340979 | C437T, P146L | 0 | 16.2 | NA | NA |
| GLTSCR2 | 19:48253494 | C349T, R117W | 0.5 | 6.8 | 0.2 | 11.2 |
| GPR160 | 3:169801777 | C17T, S6L | 0.6 | 15.7 | NA | NA |
| HLA-DMA | 6:32918428 | C241T, R81C | 0.6 | 9.9 | NA | NA |
| ICAM3^{e} | 19:10444896 | C1381T, Q461X | 0 | 18.4 | 0 | 7.2 |
| ITGB2 | 21:46319067 | C908T, S303L | 0.9 | 5.1 | NA | NA |
| LGALS9 | 17:25967659 | C193T, R65W | 0.2 | 7.6 | 0 | 5.4 |
| LRP10 | 14:23346296 | C1702T, R568X | 0 | 6.2 | NA | NA |
| NBN | 8:90955531 | C2134T, H712Y | 4 | 24.2 | 0 | 22.4 |
| PABPC4 | 1:40027426 | C1840T, H614Y | 4.4 | 31.7 | 1.6 | 39.9 |
| PCGF3 | 4:737366 | C367T, R123W | 2 | 22.2 | 0 | 12.8 |
| PPA2 | 4:106317458 | C319T, Q107X | NA | NA | 0.2 | 8.8 |
| PRPF40A | 2:153515789 | C2404T, R802X | 0 | 5.1 | NA | NA |
| RGS10 | 10:121275109 | C311T, S104L | 0.2 | 7.9 | 0 | 15.3 |
| RNH1 | 11:499165 | C464T, S155L | 3 | 18.5 | 0 | 9.2 |
| SDHB | 1:17371320 | C136T, R46X | 2.6 | 23 | 1.1 | 15.6 |
| SIN3A | 15:75668008 | C3589T, Q1197X | 0 | 17.2 | NA | NA |
| SETX^{e} | 9:135201977 | C5008T, Q1670X | 1.9 | 24.6 | NA | NA |
| SUPT6H | 17:27005584 | C1138T, R380X | 0.6 | 12.3 | NA | NA |
| TMEM109 | 11:60687274 | C109T, R37X | 0 | 11.2 | NA | NA |
| TMEM131 | 2:98409343 | C3650T, S1217L | 5.7 | 26.4 | NA | NA |
| TMEM179B | 11:62556843 | C364T, R122X | NA | NA | 0 | 5.2 |
| TRAPPC11 | 4:184585120 | C100T, R34X | 0 | 15.8 | NA | NA |
| UBE2J1 | 6:90048208 | C292T, H98Y | 4 | 16.1 | 1.1 | 18.9 |
| VIM | 10:17277300 | C1141T, R381C | 0.3 | 15.7 | NA | NA |
| XPO1 | 2:61760990 | C43T, Q15X | 2.7 | 10 | NA | NA |

^{a}NA, either editing level was not different between the two groups of samples or it could not be determined
^{b}Based on the UCSC hg19 genome assembly used for mapping reads with the Subread subjunc aligner
^{c}Nucleotide numbering for the shortest transcript isoform, with A of the ATG translation initiation codon at position 1
^{d}Calculated in analysis of RNA sequencing data (FIG. 19); mean value (n = 3)
^{e}Failed Sanger sequencing-based experimental validation

The following sequences are listed in the figures:

| | |
|---|---|
| | ID |
| Sequence | No. |

| | |
|---|---|
| AGCCTCGCCTTTGCCGA | 8 |
| TGACATCCCCCGCATCCTGG | 10 |
| CACACATATTCACTTCCAACTTTAAC | 12 |
| GGCCGAGGACCCGAAGG | 14 |
| ATGTCCGCGCAGAACAGAA | 16 |
| CTTTGACGAGACTCTACAGAAG | 18 |
| AGGCAGGAAGACCTGGCAGA | 20 |
| AAACCTGCAGATGACCAAGAC | 22 |
| AGAGAGAGCACCATTTACTG | 24 |
| CTCACCCAGGAGGGGAGAATC | 26 |
| GCAAAAGAAGTTAACAGCTGAG | 28 |
| TCAACCTCGACTCAGCGCTG | 30 |
| TTGTTGTCAGAGGCCCCA | 32 |
| GGAAGATCATCAGTCAAGGAAG | 34 |
| TGGCTGCTACCCCACTCCTG | 36 |
| AATCGGCTGGCGCAACGTCA | 38 |
| CTCAGCTCCAGTGGAACCAG | 40 |
| CACCTGCAAGCTCTATGCCAT | 42 |
| AAATCCATCTGGCATAAATGATGA | 44 |
| GGGAATGCTGCTGGAGATAG | 46 |
| TGGTACCAGGCCTCCAAG | 48 |
| GATGATGTTAAGAAGTTCAAACC | 50 |
| GAGGACATAACTCTAGAATCTG | 52 |
| GAAAATGCAAGATAAGACGCAG | 54 |
| GCTGCACCTCAGCGACAAC | 56 |
| TGTGGATAGTCTGGATAAGCT | 59 |
| TGATTACCTAGACCGAGGGC | 61 |
| ATAGCAGTGGCCTGAGAAAG | 63 |
| ATCTCAGGAGAATCGTTGGT | 65 |
| GGCGATGACTCGGACCCAG | 67 |
| TGTGACATCGTAAACATGAG | 69 |
| CCAGACTCCGATTTTGATGGAG | 71 |
| GCAGAAGAATGGTACAAATCCA | 73 |
| GCATCAGCAAGAGCAACAG | 75 |
| CTGGTGCCTGGGGCG | 9 |
| CATGTGCTGGTCATTGAGCAG | 11 |
| GTCCAGGCGCTCCACTTC | 13 |
| TTCTGACACAGGCTGCGAAG | 15 |
| CAACTTCATCCTGAATCTCCT | 17 |
| AAAGAGCACGCAGAGGTCCAG | 19 |
| GGGTACAGTGCAGACGAATC | 21 |
| AGTCATCTTTTGGCTTGGAAG | 23 |
| ATATCCCAGGAGTACACCCA | 25 |
| CGATAGCAATTGCCCTGAAATCC | 27 |
| GTTGGTTGTCCAGCAGTGACT | 29 |
| GATGTGGAAGGCATCCTGCA | 31 |
| TCCTTCCGCCTGAGCTTCTT | 33 |
| CCCAAGTATGATCAAGAATAGC | 35 |
| GCCCTATTTGCTGGATCATCC | 37 |
| CTCGTAGGTCTTCACCATCC | 39 |
| AGGATCCCGTTCACCATCAC | 41 |
| CAAGGGGAGCAGCAGAAGG | 43 |
| CTCCATTTCCTGCCTTAGCC | 45 |
| TCAAACCTTGAGTTGAATTCCATA | 47 |
| CTGCGGTGGTAGTCGTTGTC | 49 |
| ATCTTGCTTCCTCTTGAGTGCA | 51 |
| GATTTATGTCTCCTCTTCTTAC | 53 |
| CTGGTCCTGGAGTTTCTGGA | 55 |
| CTGAGCCTGGAGCTGGGGT | 57 |
| GTCAAAGTAGAGTCAACTTCAT | 58 |
| GCACATGCTCCTTGGTCCA | 60 |
| ATGCCATCAGCAAGAGGTTTGT | 62 |
| CTCTGACACCAGGTGCATGG | 64 |
| AGCAGACTGTGAAGAAGCTGCT | 66 |
| CCAAATTCACCCAAGAAGAG | 68 |
| GGAATGTACCACTCATATGAAG | 70 |
| GCTCTTCTTTCCTCAGGAGTG | 72 |
| CTGTAGGTGGCAATCTCAATG | 74 |
| TCAGTACTTCTTGAGCCATTC | 76 |
| GCAAGAGTTCCTTTTCATCTTGC | 78 |
| CTTTCATTATAAACCCGCTATAG | 79 |
| TCACCCTGTGTCCTTTGCAG | 80 |
| GAAGACTATTTGCTTTCCCTGG | 81 |
| TATGCATGATTTACCATCTTTGC | 83 |
| GTTGGCTTTGGTCCACCAC | 84 |
| CTTTCTCTTGCCACAGCAGCT | 86 |
| CCAGCAAAATGGAATTATCTTGT | 87 |
| GCACTCAGCTCACTGTGCTT | 89 |
| CAGTTGGTCCCTTTTTCAGC | 90 |
| TGCATTGTTTATTCCTCAGGC | 92 |
| CACCAGACATCTTTCTCACC | 94 |
| GTCATGGGATCAGTGGCTTAC | 77 |
| GGAGGGGATTCTTGCTCAC | 82 |
| TGAAAATAGCCACACATACGG | 85 |
| CTCTCCTTCAATAGCTGGCTT | 88 |
| AACACATGCCATCACAATGCC | 91 |
| GCTTTGTAAAAGCAACTGGGT | 93 |
| ACGCATCCAAGTCTGAGTTCC | 95 |
| GCATCCGGACAGGCATCCAA | 96 |
| GCCATGCGGACCTGCGCTTCT | 97 |
| CTGGAGCCCCAGCTTCTCCTG | 99 |
| TGATTACGACGCCCTATATAAGGAGG | 101 |
| GGCCGTAAAAGCCACAGAG | 98 |
| GAGATGAACCAAGTGACCCTG | 100 |
| TAGATGCGGGCAGCGAAG | 102 |

### EXAMPLE 2

This examples demonstrates identification of APOBEC3G as an RNA editing enzyme. In Example 1, we describe that A3A concordantly induces widespread site-specific C>U RNA editing of cellular transcripts in proinflammatory macrophages and in monocytes exposed to hypoxia and/or interferons. We also show that RNA editing function of A3A can be recapitulated by transient overexpression in 293T cells which causes site-specific RNA editing of thousands of genes (in revision). In this example, To explore whether A3G is capable of RNA editing, we transiently overexpressed it in 293T cells, performed transcriptome-wide sequencing and analysis and performed targeted experiments. We found that A3G is capable of RNA editing of a distinct set of genes, including some linked to HIV-1 replication as host factors.

RNA Seq Analysis and Verification

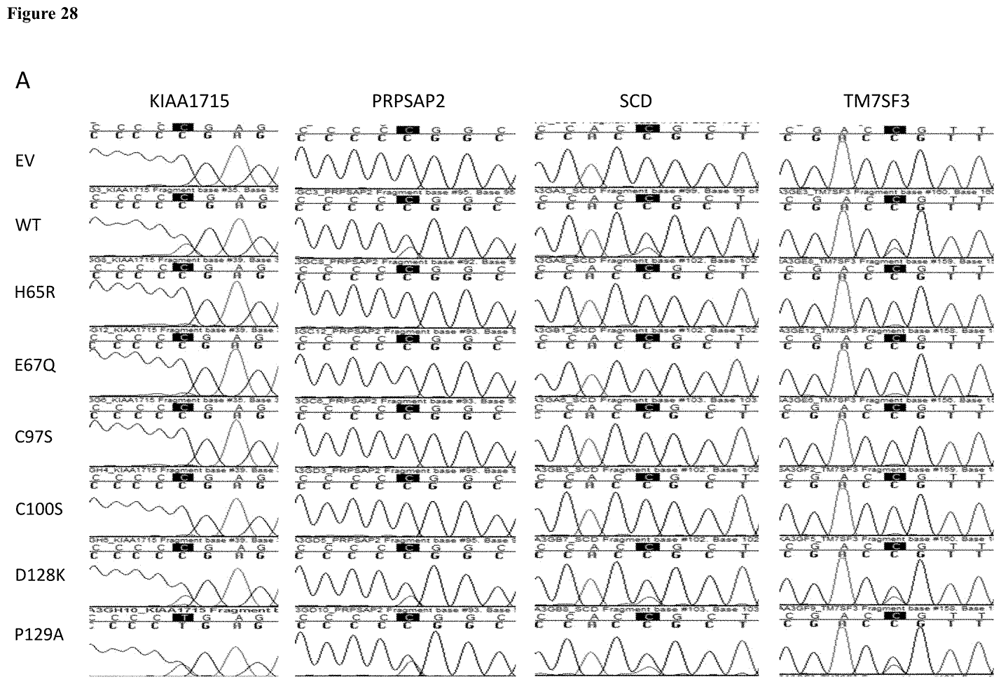

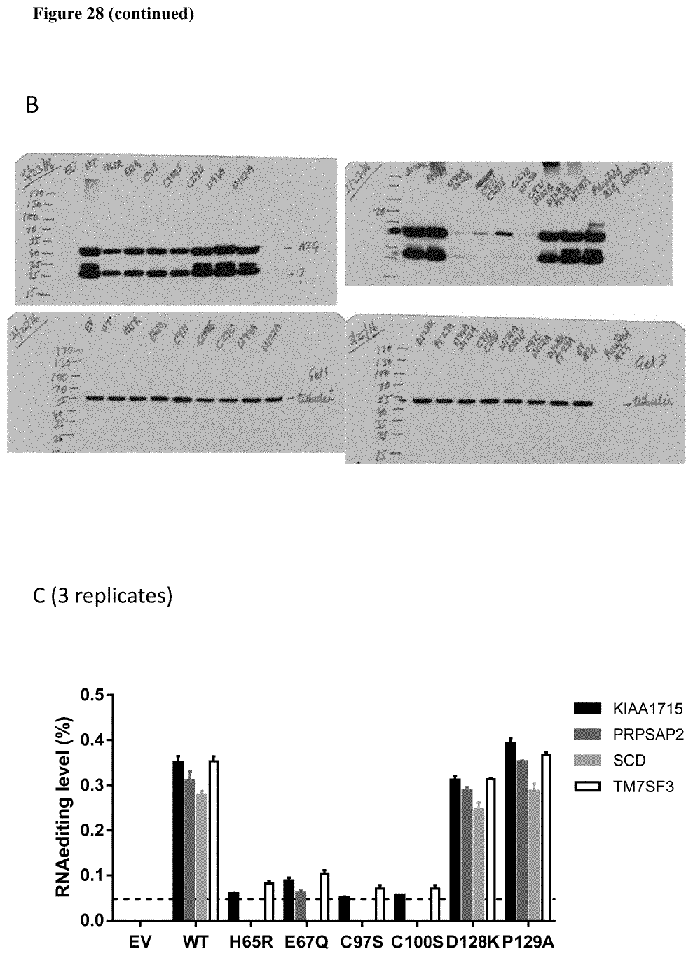

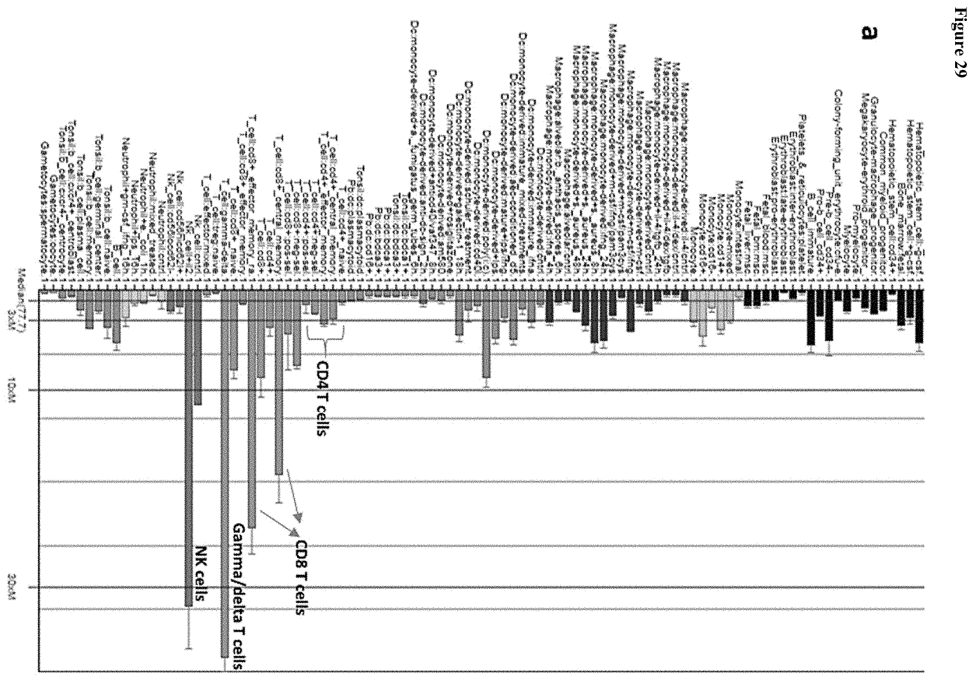

To examine transcriptome-wide RNA editing events of APOBEC3G, we transfected 1 μg of pA3G into 293T cells (293T/A3G) which caused robust protein expression (FIG. 28A). Control transfection with A3A (1 μg) showed high levels of protein expression and RNA editing as tested by one of the highly edited sites at SDHB c. 136C>U (mean RNA editing levels=50.55%±SD compared to 0.87%±SD with control empty vector, n=3). To identify transcriptome-wide RNA editing sites for A3G, we performed RNA seq approach comparing the sequences of 293T/empty vector (control) and 293T/A3G transcriptomes. 37-71 million reads were obtained for each sample in RNA sequencing. The average depth of coverage by mapped reads among the samples was at least 9 for 28-31 million genomic nucleotide positions. These positions were examined for RNA sequence variation.

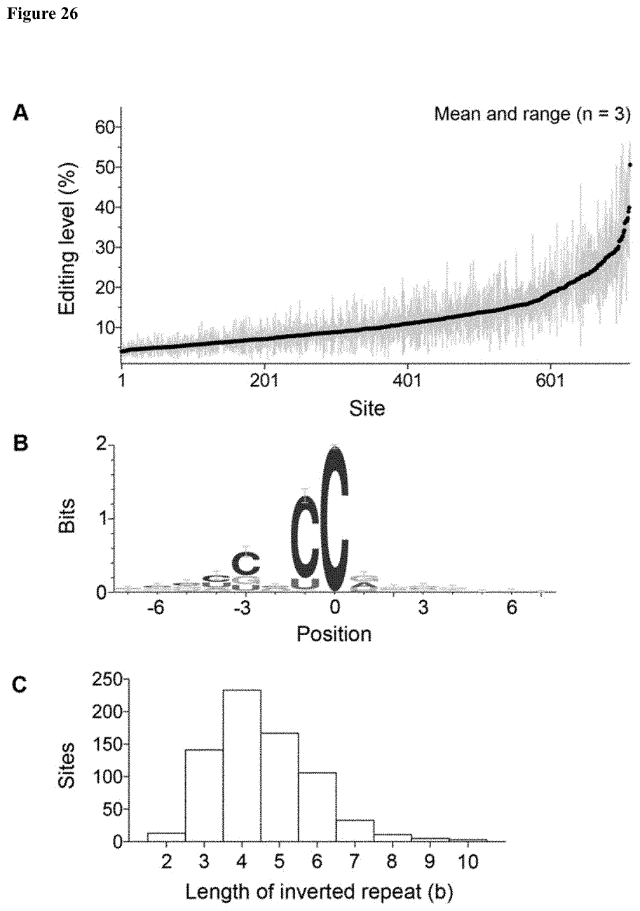

In analyses of RNA sequencing data to identify single-nucleotide sequence variations, significant differences in RNA sequences of the two groups of A3G and control transfectants were identified for 712 genomic positions. At all these positions, the sequence variation was of C>U but not any other type. Average levels of such putative C>U RNA editing were 0 in all the control samples and >5% in all the APOBEC3G transfectant samples for all 712 sites. Average C>U RNA editing levels in the A3G transfectant samples at the 712 sites were between 4% and 51% (mean=11%, SD=7%). Editing level was >20% and >30% for respectively 86 (12%) and 15 (2%) sites (FIG. 26).

690 (97%) of the 712 sites occur in the known human (RefSeq) transcriptome. Of these 692 sites, 405 (59%) are in known exonic RNA sequences (table X). C>U editing of RNA at the 712 sites is predicted to result in 174 (24%) synonymous, 173 (24%) missense and 48 (7%) nonsense changes in RNA translation (Table 2). Protein recoding RNA editing occurred in 221 sites in 217 genes. The 690 editing sites that are in the known transcriptome are transcribed for a total of 635 genes. The higher number of editing sites (4) was seen for two genes, HCFC1 and IGF2BP1. Two and 48 genes respectively had 3 and 2 editing sites (table X). Correlation between editing and gene expression levels was not observed. Notably, C>U recoding RNA editing at 27 sites in 27 genes was also catalyzed by A3A which causes such recoding of 1,100 genes in the 293T overexpression system. This finding suggests that A3A has a broader target gene profile than A3G and that RNA editing targets of these two enzymes are largely distinct.

To validate novel RNA editing sites identified by the RNA seq analysis of 293T/A3G cells, we performed Sanger sequencing of 24 new protein recoding C>U RNA editing sites in 24 genes (Table 3). These genes were chosen either because their editing levels were high enough (e.g. SCD, TM7SF3, CLASP1, PRPSAP2) to be informative in site-directed mutagenesis studies (below) or they were previously linked to HIV-1 infectivity (e.g. NMT1, CHMP4B, MPAK1) through functional studies. We validated RNA editing for 24 of 24 by Sanger sequencing in duplicate 293T/A3G transfectants, with possible exception of the ZNF142 editing which was seen only in only one replicate (FIG. 25b). With the exception of NMT1, RBM14, MED1 and MAPK1, Sanger validation experiments were performed in the same samples used in RNA seq analysis (Table 3).

To identify common features of sequence contexts of the editing sites, we examined 12 b-long sequences flanking the edited C residue. The edited C has a C, U, A or G at the immediate 5′ position for 613 (87%), 84 (12%), 9 and 6 sites, respectively. This observation and sequence logo analysis (FIG. 26c) suggests [CGU]N[CU]C[AG] as a sequence motif that is commonly targeted by APOBEC3G (The residues within brackets are different possibilities for a position. Edited C is underlined.). CCC, ACC and UCC are respectively seen for 190 (27%), 179 (25%) and 163 (23%) of the 712 editing sites. We previously noticed that edited Cs by A3A were frequently flanked by inverted repeats. Here, analysis of 25 nts containing the edited C in the middle for all edited sites by A3G shows that the edited C was flanked by a pair of inverted repeat sequences of 3-10 b for 699 (98%) of the editing sites. Sequences of 4 b are the most common, seen for 233 (32.7%) sites. Inverted repeat sequences of 4 bases or longer flanked over 75% of edited Cs by A3G, but only 28.6% in randomly obtained 25 nt sequences from human GRCh38 RefSeq transcriptome. GGC, GCC and GGCC are the three most common repeat sequences, seen for 11 (1.5%), 10 (1.4%) and 10 (1.4%) of sites, suggesting that flanking sequence complementarity rather than the sequence per se promotes editing activity. These analyses suggest that, similar to A3A, both sequence context, especially the immediate 5′-nucleotides, and the presence of long flanking inverted repeats play an important role in selection of edited Cs by A3G.

We previously noted that Cs edited by APOBEC3A are frequently located at the 3′-end of a tetra nucleotide flanked by long inverted repeats, suggesting a stem-loop structure. Here, examination of the Cs edited by APOBEC3G and confirmed by Sanger sequencing also suggests similar stem-loop structure. The Cs edited by APOBEC3G is located commonly at the 3′-end of a tetra loop flanked by inverted repeats (median=4) (Table 3).

Among the six transfectant samples of the study, 18,028 genes were considered as expressed and were analyzed for differential expression. Of the 7,582 (42.1%) genes that were differentially expressed (P<0.05), 61 and 83 were respectively down- and up-regulated with ≥2 fold-change in the APOBEC3G transfectants compared to controls.

In Vitro RNA Editing by Purified APOBEC3G

Transfection experiments that show editing of RNA but not DNA suggest that RNA is a substrate for A3G. To confirm that A3G can edit RNA in vitro, we generated 405 nt RNA sequence spanning nucleotides c.632-c.1036 of KIAA1715 by in vitro transcription and incubated it with APOBEC3G protein purified from overexpressing 293T cells. An 89 nt long ssDNA substrate containing the KIAA1715 cDNA sequence through nucleotides c.68-c.772 was included as a control. KIAA1715 mRNA acquires c.C751U mutation upon transient overexpression of A3G in 293T cells. As expected, APOBEC3G catalyzed c.C751U site-specific deamination in the RNA substrate. None of the other CC or TC sequences shows evidence of deamination by Sanger sequencing. C>T mutations were also not evident in the corresponding c.C751 site in the ssDNA template. Lack of deamination detectable by Sanger sequencing in ssDNA template is expected since A3G deaminates viral cDNA at 1%-1.5% levels which are distributed to multiple Cs {{729 Harris, Reuben S 2003; 730 Feng, Y. 2011;}}. These results suggest that certain sites in RNA are more favorable deamination substrates than ssDNA by A3G, likely owing to certain sequence/structural contexts in RNA.

A3G Site-Directed Mutagenesis for RNA Editing

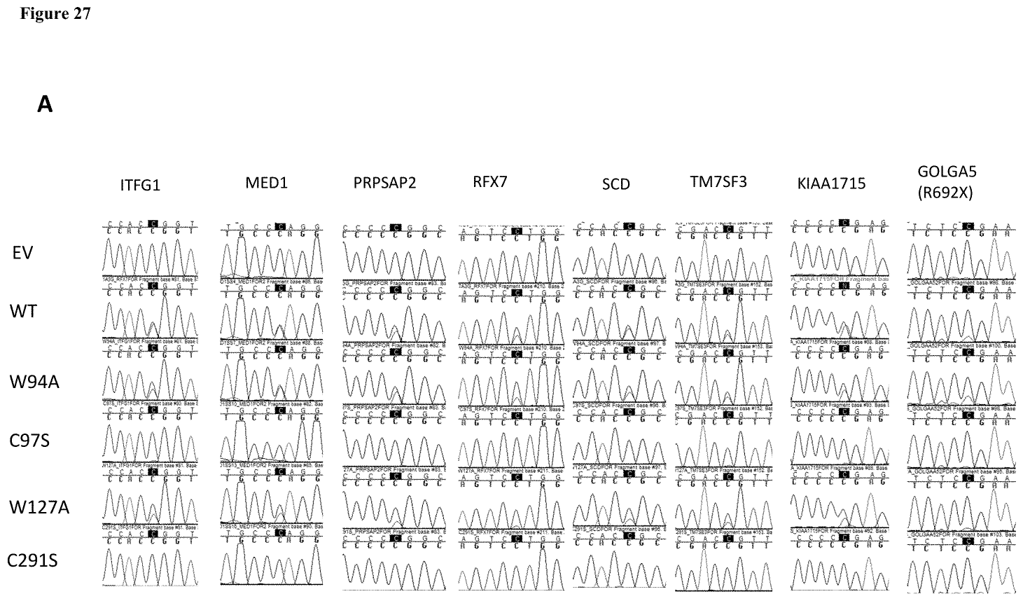

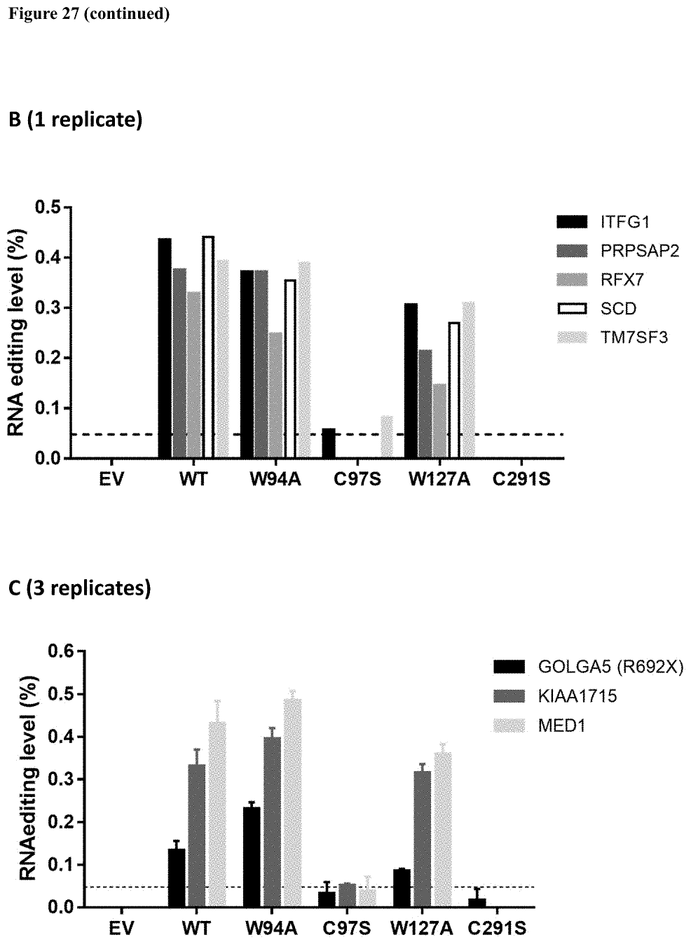

A3G NTD is involved in non-specific RNA binding but not in ssDNA deamination. To examine whether NTD is involved in RNA deamination, we initially created NTD core catalytic site mutant C97S, CTD core catalytic site mutant C291S, NTD critical RNA binding mutants W94A, W127A and C97S/C291S , W94A/W127A double mutants by site directed mutagenesis. Sanger sequencing of 293T/A3G transfectants of the mutants for eight highly edited genes showed that among single site mutants, the most dramatic reduction in RNA editing levels was observed by C97S and C291S mutants. C291S completely abolished RNA editing for all genes. C97S completely abolished RNA editing for MED1, GOLGA5 (C2074T), RFX7, PRPSAP2 and SCD but minimal residual editing was observed for ITFG1, KIAA1715 and TM7SF3. These results suggest that catalytic integrity of both NTD and CTD is essential for RNA editing, but certain RNA targets may be minimally edited even without an active NTD active site. W127A and to a lesser extent W94 mutants are reported to be essential for RNA interaction, oligomerization and virion encapsidation. We find that W127A, but not W94A, mutant impaired RNA editing (FIG. 27). Double mutant W127A/W97A abolished RNA editing completely, indicating that RNA binding by NTD is essential for cellular RNA editing.

To further examine role of the NTD conserved catalytic domain residues, we created additional mutants C100S, H65R, E67Q, D128K and P129A. These results show that N-terminal conserved catalytic residues C100, H65R and E67Q are essential for normal RNA editing (FIGS. 28). D128 and P129 residues have no role on RNA editing. The site-directed mutagenesis data indicate that both N and C terminal catalytic domains are required for RNA editing.

In Vitro RNA Editing by Purified APOBEC3G

Transfection experiments that show editing of RNA but not DNA suggest that RNA is a substrate for A3G. To confirm that A3G can edit RNA in vitro, we generated 405 nt RNA sequence spanning nucleotides c.632-c.1036 of KIAA1715 by in vitro transcription and incubated it with APOBEC3G protein purified from overexpressing 293T cells. An 89 nt long ssDNA substrate containing the KIAA1715 cDNA sequence through nucleotides c.68-c.772 was included as a control. KIAA1715 mRNA acquires c.C751U mutation upon transient overexpression of A3G in 293T cells. APOBEC3G catalyzed c.C751U site-specific deamination in the RNA substrate. None of the other CC or TC sequences shows evidence of deamination by Sanger sequencing. C>T mutations were also not evident in the corresponding c.C751 site in the ssDNA template. Lack of deamination detectable by Sanger sequencing in ssDNA template is expected since A3G deaminates viral cDNA at 1%-1.5% levels which are distributed to multiple Cs. These results suggest that certain sites in RNA are more favorable deamination substrates than ssDNA by A3G, likely owing to certain sequence/structural contexts in RNA.

RNA Editing by APOBEC3G Targets Cellular Genes Involved in HIV-1 Infection

We noted that some host genes known to be involved in HIV infectivity are targeted for RNA editing. These genes include ACIN1, CHMP4B, SIN3A, subunit genes of mediator complex MED (MED1, MED28), NFAT5, NMT1, RBM14 and MAPK1 MED 1, which encodes a subunit of SP1/mediator complex is implicated in HIV-1 replication in several studies. Other edited genes that belong to cellular pathways important for HIV-1 infectivity (e.g. NF-KB, ESCRT, chromatin modifications) are also noted (Table 4). RNA editing of host genes linked to HIV-1 infectivity by previous functional studies suggests that A3G may alter the host environment to antagonize HIV-1 infection. The antagonistic effect of RNA editing may involve reducing the amount or quality of the accessory host proteins that are critical for HIV-1 life cycle. Alternatively, but not exclusively, RNA editing of host genes might facilitate virion encapsidation of A3G by modifying the intracellular protein trafficking pathways.

TABLE 2
Gene features and effects on translation codon
for APOBEC3G-mediated C < U RNA editing sites^{a}

| 5′ untranslated region | | | 39 |
|---|---|---|---|
| Exonic | Synonymous | | 174 |
| | Non-synonymous | Nonsense | 48 |
| | | Stop loss | 0 |
| | | Missense | 173 |
| | | Unknown | 2 |
| | Non-coding RNA | | 8 |
| 3′ untranslated region | | | 227 |
| Intronic | Coding RNA | | 12 |
| | Non-coding RNA | | 7 |

| | |
|---|---|
| Untranscribed^{b} | 10 |
| Intergenic | 12 |

^{a}As reported by the ANNOVAR annotation tool
^{b}Within 1 kb up- or down-stream respectively of a known transcription start or endsite

TABLE 3
Sanger validation of selected C > U recoding RNA editing sites identified in 293T/A3G cells

| | | | RNA | | Sizes (of loop |
|---|---|---|---|---|---|
| | | | editing | RNA | (N . . . C)/ |
| | | | level | editing | immediately |
| | Chromosomal | | (RNA | level | flanking |
| Gene^{a} | position^{b} | cDNA and amino acid change^{c} | seq) | (Sanger) | palindrome ^{d} |
| ACINI | 14:23063490 | NM_001164816:exon6:c.C676T: | 0.22 | 0.23 | 4/5 |
| | | p.Q226X | | | |
| CDC6 | 17:40291480 | NM_001254:exon4:c.C472T: | 0.08 | 0.14 | 3/6 |
| | | p.Q158X | | | |
| CHMP4B | 20:33850995 | NM_176812:exon3:c.C412T: | 0.07 | 0.09 | 4/5 |
| | | p.Q138X | | | |
| CLASP1 | 2:121469844 | NM_001142273:exon9:c.C829T: | 0.26 | 0.45 | 3, 4/2 |
| | | p.R277W | | | |
| GOLGA5 | 14:92809464 | NM_005113:exon4:c.C937T: | 0.09 | 0.19 | 4/4 |
| | | p.Q313X | | | |
| GOLGA5 | 14:92837408 | NM_005113:exon12:c.C2074T: | 0.10 | 0.13 | 3/3 |
| | | p.R692X | | | |
| ITFG1 | 16:47155742 | NM_030790:exon18:c.C1816T: | 0.16 | 0.38 | 3/2 |
| | | p.R606W | | | |
| KIAA1715 | 2:175939613 | NM_030650:exon10:c.C751T: | 0.26 | 0.34 | 4/5 |
| | | p.R251X | | | |
| MAPK1 | 22:21788373 | NM_002745:exon6:c.C740T: | 0.13 | 0.11 | 4/3 |
| | | p.P247L | | | |
| MED1 | 17:39410258 | NM_004774:exon17:c.C1963T: | 0.27 | 0.38 | 4/6 |
| | | p.Q655X | | | |
| NFAT5 | 16:69693268 | NM_006599:exon12:c.C3389T: | 0.12 | 0.12 | 4/2 |
| | | p.S1130L | | | |
| NFRKB | 11:129873843 | NM_006165:exon20:c.C2527T: | 0.07 | 0.09 | 7/5 |
| | | p.Q843X | | | |
| NMT1 | 17:45061373 | NM_021079:exon1:c.C44T: | 0.23 | 0.19 | 4/6(i + 1) |
| | | p.P15L | | | |
| NVL | 1:224289696 | NM_001243146:exon11:c.C796T: | 0.06 | 0.09 | 4/4 |
| | | p.Q266X | | | |
| PRPSAP2 | 17:18928928 | NM_001243936:exon9:c.C802T: | 0.25 | 0.35 | 4/4 |
| | | p.R268W | | | |
| RBM14 | 11:66626504 | NM_006328:exon3:c.C1846T: | 0.22 | 0.26 | 4/2 |
| | | p.R616C | | | |
| RFX7 | 15:56096472 | NM_022841:exon9:c.C1256T: | 0.29 | 0.29 | 4/3 |
| | | p.P419L | | | |
| SCD | 10:100352431 | NM_005063:exon3:c.C376T: | 0.24 | 0.33 | 4/4 |
| | | p.R126C | | | |
| SGPL1 | 10:70854801 | NM_003901:exon5:c.C355T: | 0.05 | 0.11 | 4/4(i + 1) |
| | | p.Q119X | | | |
| SUCLA2 | 13:47973266 | NM_003850:exon5:c.C661T: | 0.13 | 0.17 | 4/6 |
| | | p.Q221X | | | |
| TM7SF3 | 12:26974149 | NM_016551:exon12:c.C1529T: | 0.30 | 0.39 | 4/2 |
| | | p.P510L | | | |

TABLE 4
Genes that undergo C > U recoding RNA editing by APOBEC3G regulate cellular pathways involved in HIV-1 infection.

| | | | | Interacts | | | | | | |
|---|---|---|---|---|---|---|---|---|---|---|
| ESCRT | | | | with | | | | | | |
| pathway, | RNA, DNA | | | HIV | Nuclear | | | | | |
| HIV RNA | replication, | NF-KB | Chromatin | RNA/ | membrane/ | Cyto- | Mito- | | MAPK | |
| trafficking | modification | pathway | modifiers | proteins | pore | skeleton | chondria | Proteasome | signaling | Golgi |
| ZNF142 | MED1, | USP34 | PAXIP1 | ACIN1 | LBR | PDLIM3 | DNM1L | PSMD4 | LAMTOR3 | GOLGA3, |
| | MED15, | | | | | | | PSMC3 | | GOLGA5 |
| | MED28 | | | | | | | | | |
| CHMP4B | PAPOLG | TAB1 | NFRKB | WDR48 | NUP205 | CLASP1 | SUCLA2 | | MAPK1 | |
| MID2 | CDC6 | UBE2L3 | CTCF | NSUN5 | NUP54 | CAMSAP1 | | | | |
| RBM14 | SETD2 | UBE2N | SMARCA4 | NMT1 | | | | | | |
| | | (UBC13) | | | | | | | | |
| VPS37A | NFAT5 | | KMT2A, | HSPA5 | | | | | | |
| | | | KMT2C, | | | | | | | |
| | | | KMT2D | | | | | | | |
| ATRN | NEIL3 | | BAZ1B | | | | | | | |
| AP2M1 | HUWE1 | | CHD7, | | | | | | | |
| | | | CHD8 | | | | | | | |
| SIN3A | EIF31 | | CBX6 | | | | | | | |
| SMG6 | | | ARID4A | | | | | | | |
| | | | PHF2 | | | | | | | |
| | | | ATF7IP | | | | | | | |
| | | | JADE1 | | | | | | | |
| | | | ASH1L | | | | | | | |
| | | | EP300 | | | | | | | |

### EXAMPLE 3

This examples demonstrates that APOBEC3G is involved in RNA editing in natural killer cells and cancer cells and that inhibiting APOBEC3G in cancer cells stops their proliferation. In Example 1, we describe that A3A concordantly induces widespread site-specific C>U RNA editing of cellular transcripts in proinflammatory macrophages and in monocytes exposed to hypoxia and/or interferons. We also show that RNA editing function of A3A can be recapitulated by transient overexpression in 293T cells which causes site-specific RNA editing of thousands of genes (in revision). In example 2 we show that APOBEC3G is an RNA editing exzyme. In this example, we examine whether A3G is involved in RNA editing of immune cells and cancer cells. We demonstrated that A3G is involved in RNA editing in natural killer cells and in lymphoma cells. Inhibition of RNA editing in lymphoma cells stops cancer proliferation.

Results

Cell Type Specific Expression of APOBEC3G

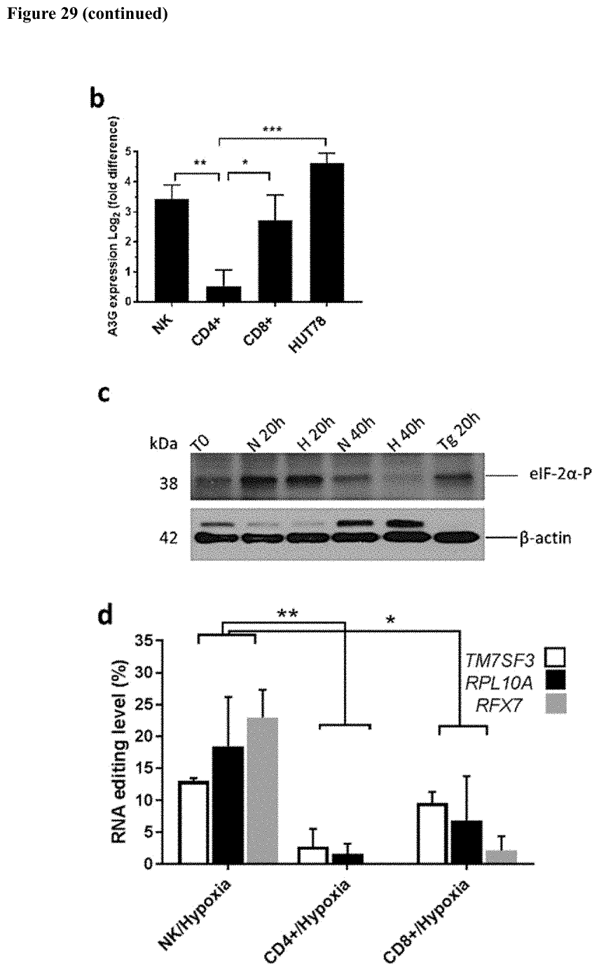

To examine the endogenous RNA editing activity of A3G, we first analyzed A3G's cell type specific expression levels. A meta-analysis of the publicly available microarray datasets (Abbas et al., Genes Immun 2005, 6:319-331) indicated high expression of A3G in gamma delta T cells, NK cells and CD8+ T cells (in that order, FIG. 29a). We experimentally confirmed high expression levels of A3G in primary NK and CD8+ T cells, but found lower expression in primary CD4+ T cells purified from peripheral blood (FIG. 29b). These results are unexpected because prior studies have implied a potential functional role of A3G in restricting HIV-1 in infected CD4+ T cells (Kreisberg et al., J Exp Med 2006, 203:865-870; Vetter et al., PLoS Pathog 2009, 5:e1000292). In contrast, our findings reveal the highest expression of A3G in NK and CD8+ T lymphocytes that are not infected by HIV-1.

Identification of RNA Editing by APOBEC3G in NK Cells

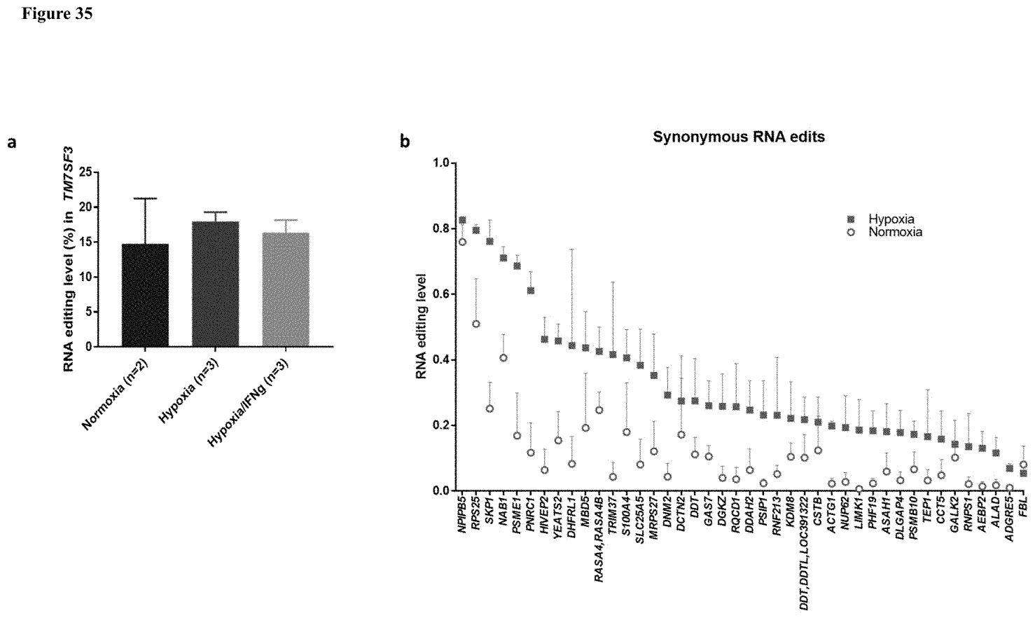

We have previously shown that A3A, which is highly expressed in monocytes and macrophages shows very low or the absence of RNA editing in these cells when freshly isolated from peripheral blood mononuclear cells (PBMCs) (Sharma et al., Nat Commun 2015, 6:6881). However, RNA editing is induced when monocytes/macrophages are cultured at a high cell density and low oxygen (hypoxia, 1% O₂) or by interferons (Sharma et al., Nat Commun 2015, 6:6881; Baysal et al., PeerJ 2013, 1:e152). We cultured PBMCs for 40 hours at a high cell density (5×10⁷ cells in 1.8 ml per well in a 12-well plate) under normoxia or hypoxia and isolated NK cells. Under these conditions, we observed upregulation of the phosphorylated α subunit of the eukaryotic initiation factor-2 (eIF-2α) at Ser 51-a conserved event activated in response to various cell stresses including hypoxia at 20 h, suggesting that NK cells were stressed (FIG. 29c). To examine site-specific C>U editing in RNAs of NK cells, we selected several candidate genes including TM7SF3 that we have previously shown high-level RNA editing on overexpressing A3G in 293T cells (Sharma et al., Sci Rep 2016, 6:39100). We found evidence for induction of RNA editing in TM7SF3 due to cellular crowding with/without hypoxia (higher in hypoxia), which did not further increase with IFN-γ treatment (FIG. 35a). Since A3G is also expressed in CD8+ T cells and to a lesser extent in CD4+ T cells (FIG. 29a), we cultured PBMCs as mentioned above and isolated NK, CD8+ and CD4+ cell subsets from the same donors. Site-specific RNA editing (>5%) was observed in NK cells and to a lesser extent in CD8+ T cells, but not in CD4+ T cells (FIG. 29d), in parallel with the relative expression levels of A3G in these cell types. Our results indicate that A3G induces RNA editing in cytotoxic lymphocytes, particularly in NK cells.

RNASeq Analysis of NK Cells

To determine the transcriptome-wide targets of C>U RNA editing and their respective editing level in NK cells, we performed RNASeq analysis. PBMCs (n=3 donors) were cultured at a high density with/without hypoxia (1% O₂) and site-specific editing of TM7SF3 RNA was first confirmed, which showed higher level of editing in hypoxia relative to normoxia. The three normoxic and three hypoxic NK cells RNA samples were then sequenced by following the TruSeq RNA Exome protocol (see methods). Analysis of the RNASeq results was based on all C>U editing events in exons and UTRs that were (a) at least 5% in any sample, (b) overrepresented in the hypoxia group and (c) located in a putative RNA stem-loop structure (see methods for details).

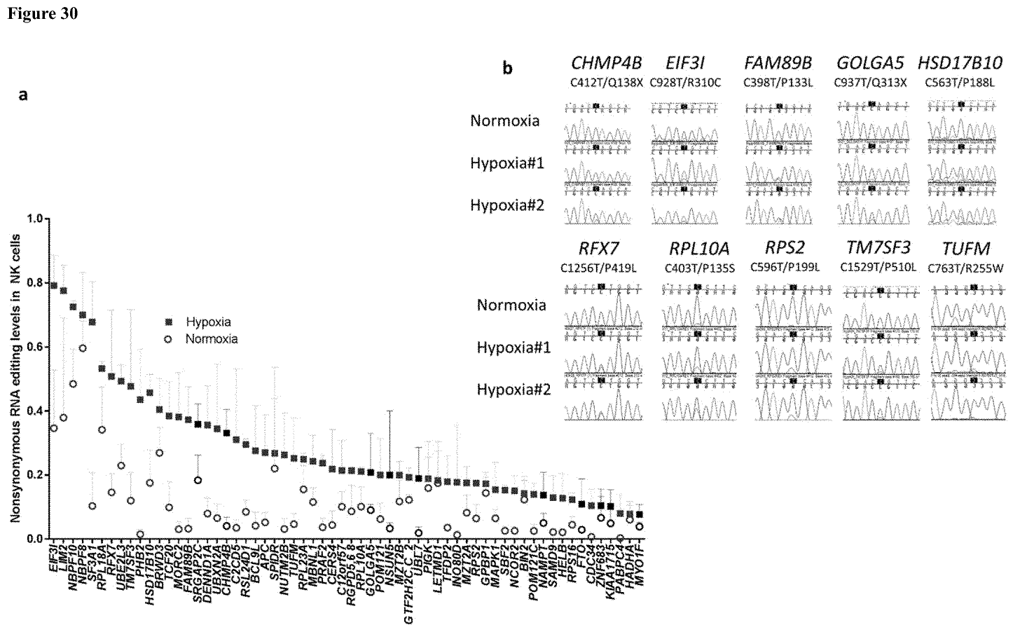

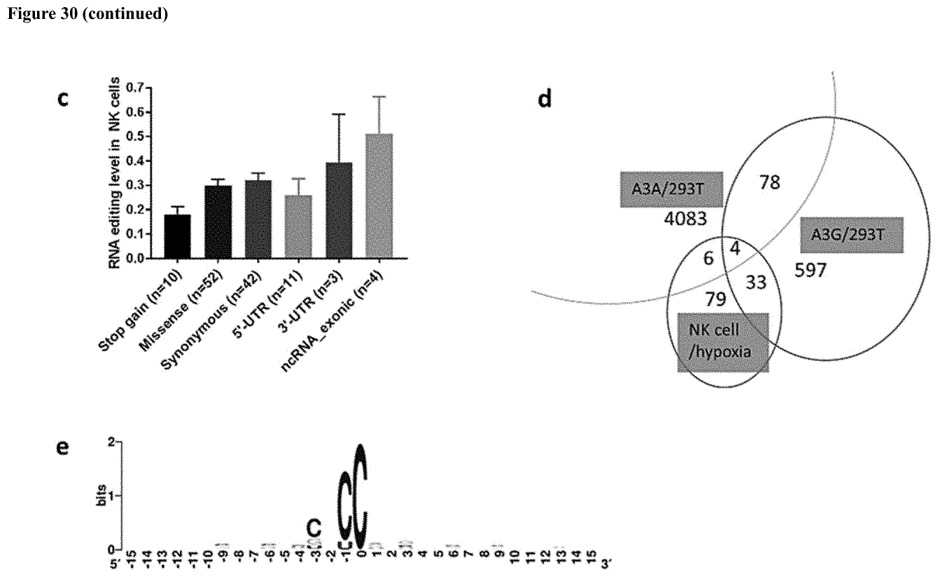

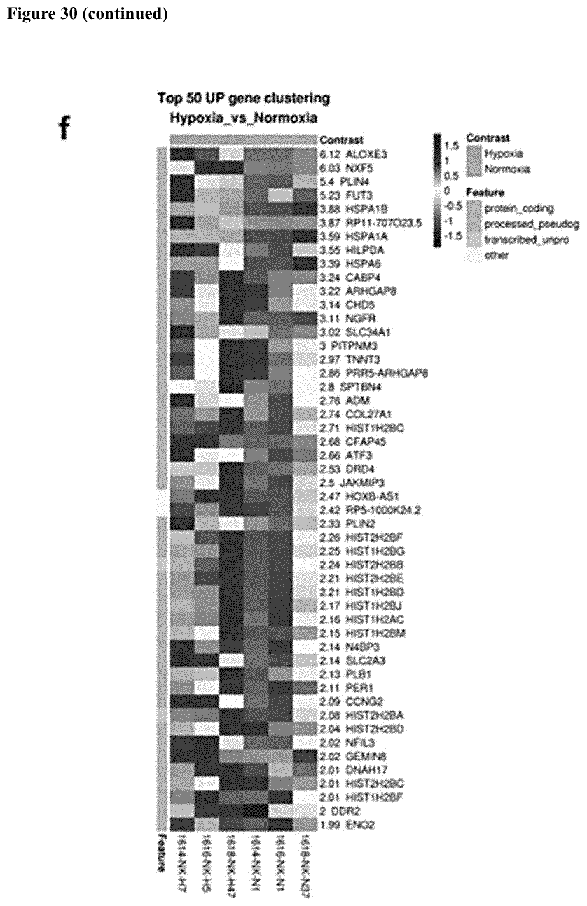

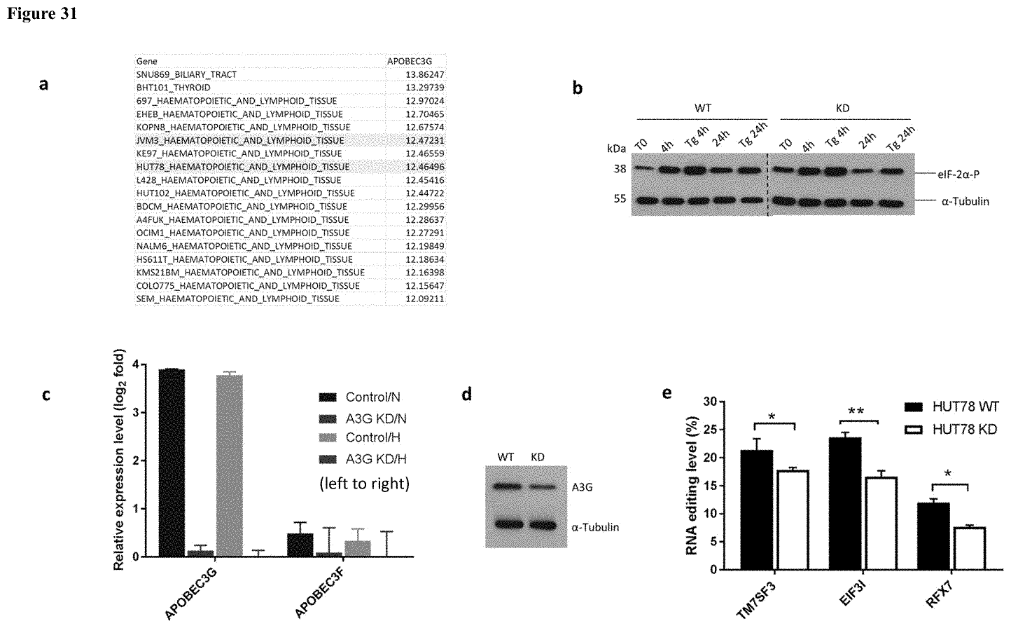

RNASeq analysis revealed 122 site-specific C>U editing events which were edited at a higher level in hypoxia as compared to normoxia, although editing also occurred in normoxia at variable levels due to cellular crowding in NK cells. The largest group of editing events comprised of non-synonymous changes, including 52 missense and 10 stop gain changes (FIG. 30a). Synonymous C>U editing events occurred in RNAs of 42 genes (FIG. 35b). We verified RNA editing by Sanger sequencing of cDNAs in 10 of 10 non-synonymously edited genes, which include CHMP4B, EIF3I, FAM89B, GOLGA5, HSD17B10, RFX7, RPL10A, RPS2, TM7SF3 and TUFM (FIG. 30b). The highest level of non-synonymous RNA editing (˜80%) occurred in EIF3I, which alters a highly conserved arginine to cysteine (c.C928T; R310C) (FIGS. 30a and b). The average editing levels were lower for stop gain changes than for missense or synonymous changes and for changes in the UTRs and nc_RNA exonic sites, suggesting functional constraints on editing events that introduce stop-gain changes (FIG. 30c).

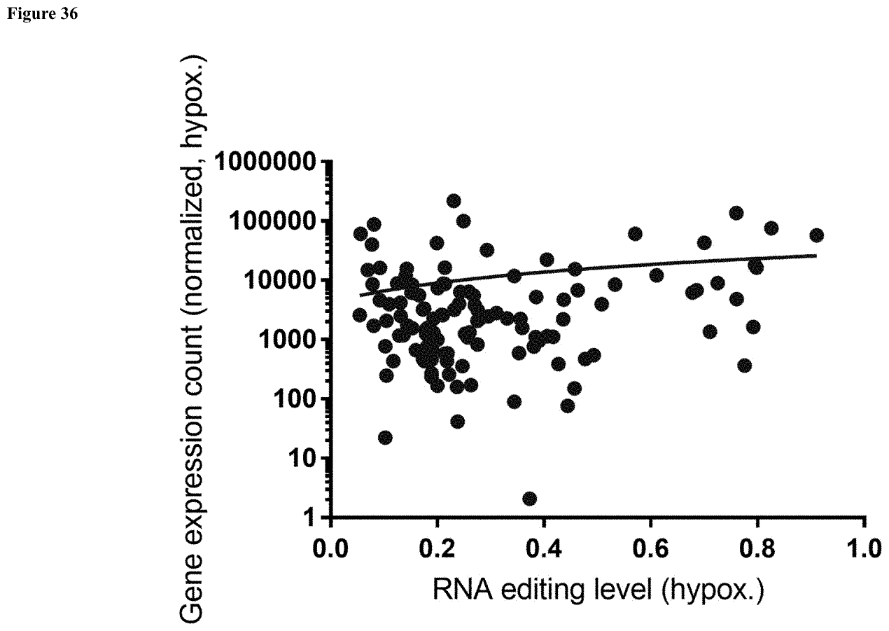

We identified that 37 of the 122 RNA editing sites in NK cells were among the 712 sites (exons+UTRs) in the 293T/A3G system whereas only 10 edited sites in NK cells were among the 4,171 sites in the 293T/A3A system (p=10⁻⁵, Fisher's exact test) indicating that A3G is more likely to catalyze RNA editing in NK cells than A3A (FIG. 30d). Interestingly, 85 edited sites identified in NK cells did not overlap with those in the 293T/A3G system. Different parameters used for the computational analysis of edited sites, cell type specific factors and the method of induction of RNA editing (overexpression versus hypoxia) may play a role in the differences observed in the RNA editing targets of A3G in primary cells versus its exogenous overexpression in 293T cells. A3G has a preference for CC nucleotides both in its ssDNA and RNA substrates, whereas other A3 enzymes are known to prefer TC nucleotides. Sequence motif analysis of the 122 editing sites in NK cells shows a strong preference for C at −1 position (FIG. 30e), suggesting that these RNA editing events are catalyzed by A3G. The level of RNA editing and the expression of genes whose RNAs undergo editing show a weak positive correlation which is not statistically significant (r=0.1695, p=0.0620, n=122 genes, FIG. 36), suggesting that expression levels of the RNA edited genes do not influence RNA editing levels.

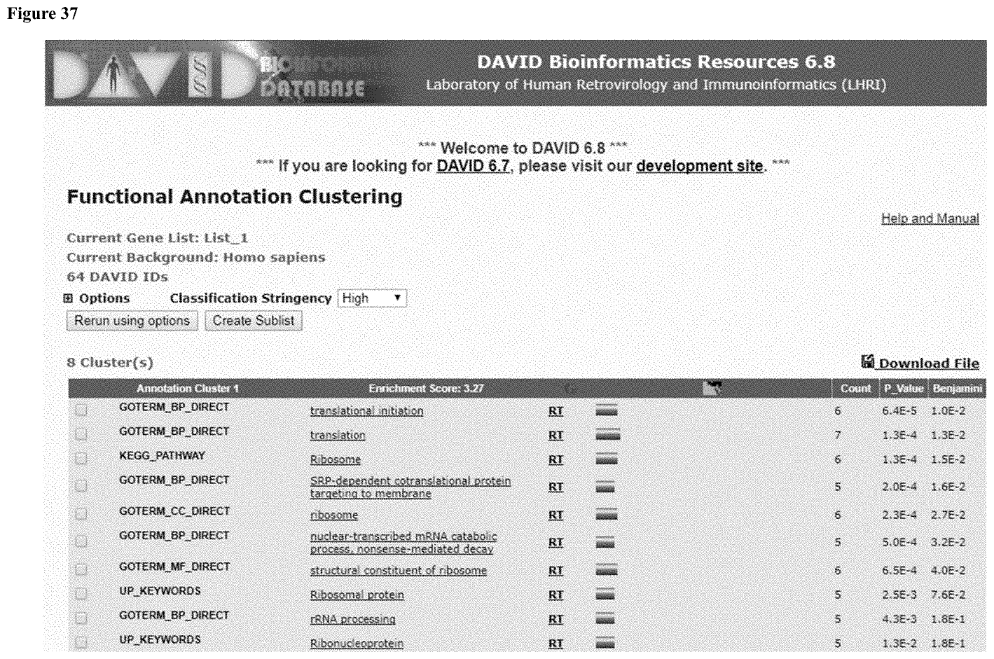

We determined the functional clustering of genes that undergo non-synonymous changes (n=62) due to RNA editing in NK cells using DAVID Bioinformatics Resources. The highest enrichment was for genes involved in “translation initiation”, “translation” and “ribosome” (FIG. 37) due to missense changes in RNAs of 8 genes (Table 5), including the highest non-synonymously edited EIF3I (FIG. 30a). RNA editing targeted highly conserved amino acids in 7 of 8 genes as predicted by at least 2 of the 3 software programs (Table 5) for conservation analysis of all non-synonymous RNA editing sites). RNA editing also altered a conserved C (phyloP100 score=1.7811) at −4 nucleotide position in the 5′-UTR of another gene encoding the ribosomal protein, RPLP0. Since the regulation of translation plays a central role during cell stress, these results suggest that RNA editing coordinately alters multiple ribosomal and other translational proteins, and may have an impact on the quality or quantity of protein translation under hypoxic stress.

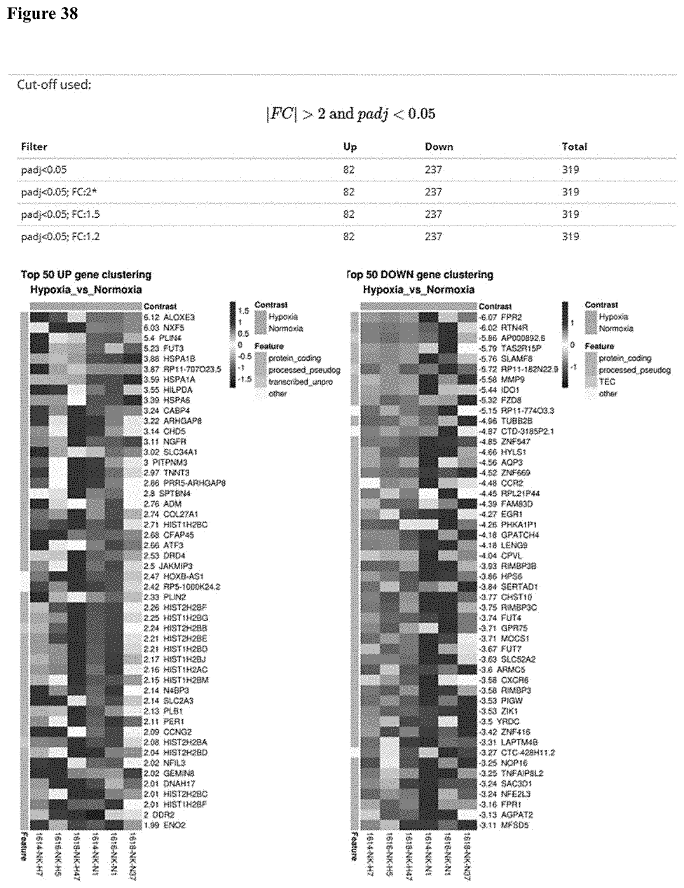

We also examined the changes in gene expression that occur during the induction of RNA editing in NK cells due to cellular crowding and hypoxia. We found upregulation of 82 genes and downregulation of 237 genes (fold change>2 and padj<0.005, FIG. 38). Multiple genes of the heat shock protein HSP70 family (HSPAJB, HSPAJA, HSPA6) and ATF3, which encodes a transcription factor integral to the ER stress response are among the most upregulated (FIG. 30f). Thus, cellular crowding and hypoxia triggers a coordinated transcriptome remodeling in NK cells, which includes transcriptional induction of stress genes as well as recoding C>U RNA editing of translational and ribosomal genes.

Confirmation of APOBEC3G-Mediated RNA Editing in Lymphoma Cell Lines

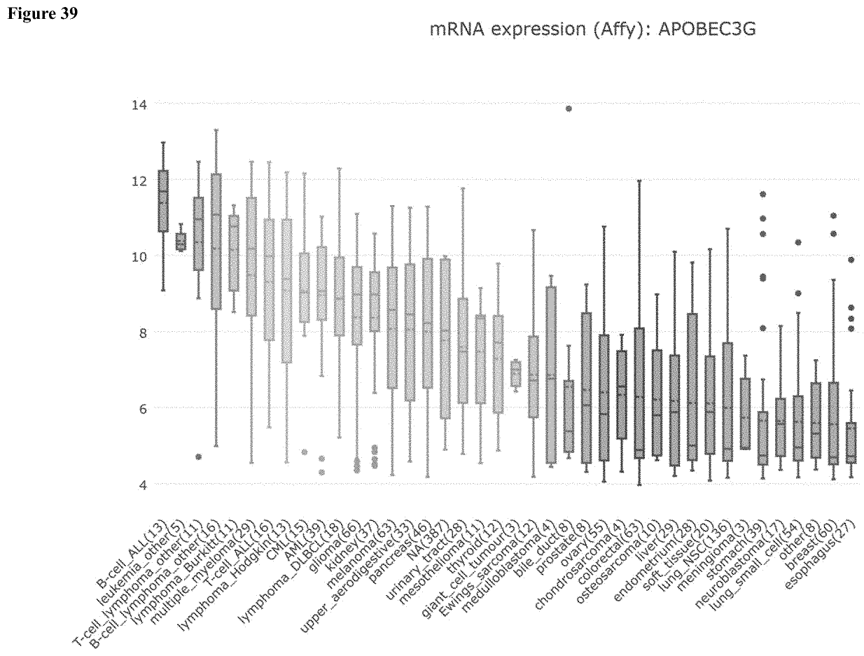

To confirm A3G-mediated RNA editing and to examine the functional consequence of this editing in a cell line, we searched for cell lines that express A3G. We first examined the relative expression of A3G in silico in more than a 1,000 cell lines at the CCLE database. The highest expression of A3G was observed in leukemia and lymphoma cell lines (FIG. 39). Next, we ranked cell lines in the order of highest to lowest A3G expression (FIG. 35a) and selected JVM3 (rank=6), an EBV-transformed B cell prolymphocytic leukemia cell line, and HuT78 (rank=8), a CD4+ cutaneous T cell lymphoma cell line.

As compared to primary CD4+ T cells, A3G is highly expressed in the HuT78 lymphoma cell line (FIG. 29b and FIG. 35a). To further validate the RNA editing function of A3G, we knocked-down A3G in these cells using an A3G-specific shRNA lentiviral construct and a scramble negative control shRNA (referred to as WT HuT78). The WT HuT78 cells and the A3G knock-down cell line (KD) were further propagated and cultured at a high density of 1×10⁶ cells in 100 μl per well in a 96 well plate for 24 hours in normoxia or hypoxia (1% O₂). High density culture and/or hypoxia treatment induced cell stress as there was an increased accumulation of phosphorylated eIF-2α, 4 h post culture (FIG. 35b). Under these conditions, we measured the expression of A3G in these cell lines by qPCR (FIG. 35c). The KD HuT78 cells showed markedly reduced expression of A3G as compared with WT HuT78 (FIG. 35c). We did not observe any significant variation in A3G levels with or without hypoxia treatment in the WT and KD HuT78 cells. A3F, which is also expressed in HuT78 did not show any significant variation in expression level between the WT and KD HuT78 cell lines, indicating that the knockdown for A3G is specific (FIG. 35c). We further confirmed the knock down of A3G by analyzing its protein expression by western blot (FIG. 35d) using specific antibodies to A3G. As compared to WT, KD HuT78 cells showed a reduction in A3G expression (FIG. 35d).

To determine the effect of A3G knock-down on RNA editing, we analyzed the editing level of three RNAs (TM7SF3, EIF3I and RFX7) previously validated as editing targets in NK cells. When cultured at a high density (mentioned above), we found site-specific editing of TM7SF3, EIF3I and RFX7 RNAs in WT HuT78 cells and the level of editing was reduced in the A3G KD HuT78 cells (FIG. 35e), correlating with the expression of A3G in these cells.

Considering that (1) A3G has a CC nucleotide preference, (2) RNA editing targets in NK cells and in 293T/A3G overexpression system overlap significantly (3) the same RNAs are site-specifically edited in NK and HuT78 cells-both highly expressing A3G; and (4) A3G KD HuT78 cells show decreased RNA editing, these results collectively indicate that A3G is an endogenous, inducible mRNA editing enzyme in NK, CD8+ and HuT78 (and JVM3) cells.

A3G Induces RNA Editing by Mitochondrial Respiratory Inhibition, Independently of HIF-1α

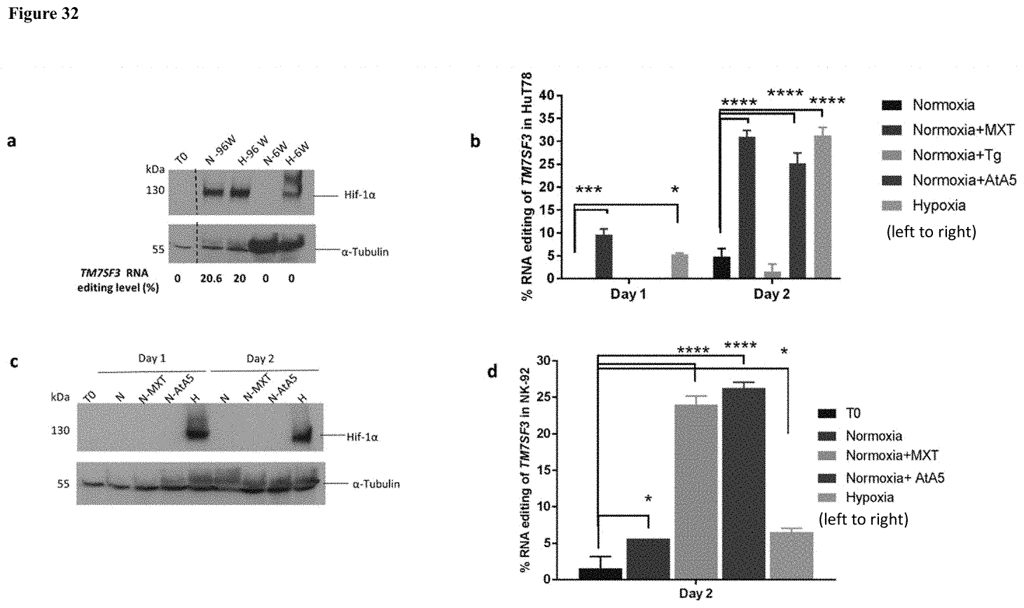

To determine whether high density of HuT78 cells, which induces RNA editing by A3G, causes hypoxia, we cultured 1×10⁶ HuT78 cells in 100 μl per well in 96 well plates (high density) and the same number of cells in 1 ml culture in 6 well plates (low density), each under normoxia and hypoxia. We analyzed the stabilization of the hypoxia-inducible factor-1α (HIF-1α) protein, which is well known to be stabilized in hypoxic cells to promote the synthesis of mRNAs involved in cellular homeostasis, and measured the RNA editing levels of TM7SF3. As expected, HIF-1α was not stabilized at T0—when the cells were at a non-stressed state or under low density normoxic cell culture (6 well) after 24 hours (FIG. 32a). However, we found the stabilization of HIF-1α in cells cultured at a high density in 96 well plates both in normoxia and hypoxia, and in cells cultured at a low density in 6 well plates in hypoxia, suggesting that the high density 96 well normoxic culture had turned hypoxic (FIG. 32a). Under these conditions, RNA editing of TM7SF3 was observed in cells cultured at a high cell density in both normoxia (20.6%) and hypoxia (20%) (FIG. 32a). Although HIF-1α stabilization was observed in low cell density (6 well) hypoxic cultures, no RNA editing was observed under these conditions (FIG. 32a). These results confirm that as in NK cells RNA editing is induced by high cell density and hypoxia in HuT78 cells. Moreover, the stabilization of HIF-1α is not sufficient for the induction of RNA editing.

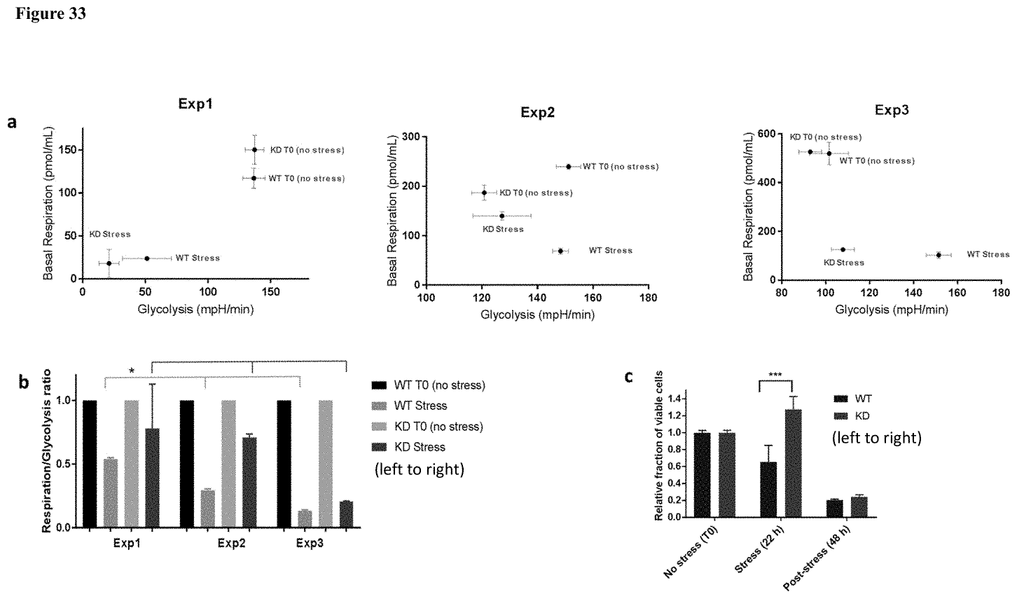

We tested the effect of inhibitors of mitochondrial complex II by atpenin A5 (AtA5) and of the complex III by myxothiazol (MXT) on RNA editing in HuT78 cells cultured in normoxia. Additionally, to test whether endoplasmic reticulum (ER) stress can also induce RNA editing, we treated the cells with Thapsigargin (Tg). Tg is considered to induce ER stress by raising intracellular calcium levels and lowers the ER calcium levels by specifically inhibiting the endoplasmic reticulum Ca⁺⁺ ATPase, resulting in the accumulation of unfolded proteins and an increased accumulation eIF-2α phosphorylated at Ser 51 (FIGS. 33c and 35b). To test the effect of hypoxic stress alone on HuT78 cells, we reduced the cell density to avoid cellular crowding and cultured the cells at an intermediate density of 0.5×10⁶ cells per 500 μl per well in 24 well plates with or without the chemical inhibitors in normoxia, and hypoxia alone for one or two days. Under these conditions, we determined RNA editing level and the stabilization of HIF-1α in these cells. We observed that RNA editing is mildly induced in cells treated with MXT and by hypoxia alone on day 1, at approximately 10% and 5% levels, respectively (FIG. 32b). RNA editing levels increased to approximately 30% in cells treated with MXT, AtA5 or hypoxia alone on day 2. Treatment of cells with Tg did not induce RNA editing (FIG. 32b). Furthermore, HIF-1α was stabilized only when the cells were subjected to hypoxia but not in normoxia in the presence or absence of the mitochondrial inhibitors (FIG. 32c). These results suggest that RNA editing induced by hypoxic stress at a high cell density is triggered by mitochondrial respiratory inhibition and occurs independently of the stabilization of HIF-1α as well as the ER stress response.

Although the A3G expression data did not include the NK-92 lymphoma cell line in the CCLE database, given its similar characteristics to primary NK cells and the convenience of culturing NK-92 cells as compared with primary NK cells, we tested the induction of RNA editing in NK-92 cells. We treated NK-92 cells with normoxia with or without the mitochondrial inhibitors (AtA5 or MXT) or hypoxia alone at intermediate density in 24 well plates for 2 days. Interestingly, RNA editing was induced by the inhibition of mitochondrial respiration (˜25%), but only slightly by hypoxia treatment (FIG. 32d) in NK-92 cells. The reason behind the difference in hypoxia induced RNA editing level of HuT78 and NK-92 cells may be due to metabolic differences between the two cell lines.

APOBEC3G promotes Warburg-like metabolic remodeling and suppresses proliferation under stress

In the current study, we find that A3G non-synonymously edits several mitochondrial genes' RNAs including TUFM, HADHA, HSD17B10 and PHB2 in hypoxic NK cells (FIG. 30a). Thus hypoxic stress-induced RNA editing by A3G appears to alter mitochondrial function.

To test the role of A3G on bioenergetics in response to high cell density and hypoxic stress, we measured the metabolic profile of WT and KD HuT78 cells using the Seahorse platform. We performed the mitochondrial and the glycolytic stress tests to measure the oxygen consumption rate, representative of basal respiration and the extracellular acidification rate, representative of glycolysis in cells cultured at a high density in three separate experiments (FIG. 33a). We have presented metabolic alterations as respiration-to-glycolysis ratio (R/G) both in unstressed (T0) and stressed cells (FIG. 33b). As expected, cell stress caused by high cell density reduced R/G ratio in each experiment relative to unstressed TO cells, indicating a decrease in respiration relative to glycolysis. However, R/G ratios decreased to a lesser extent under stress in A3G KD, relative to WT HuT78 cells, indicating that A3G plays a role in reducing mitochondrial respiration relative to glycolysis under hypoxic stress caused by high cell density (FIG. 33b).

Hypoxic stress can suppress translation and lead to growth arrest by inhibiting cell cycle progression in non-transformed cells or by promoting apoptosis by the p53 pathway in transformed cells. To examine the role of A3G on cellular proliferation under stress, we measured the proliferation of the WT and KD HuT78 cells when cultured at a high density for 22 hours followed by ‘recovery period’ by culturing these stressed cells at a low density for another 48 hours. The fraction of viable cells reduced in WT, but increased in A3G-KD HuT78 cells during 22 hours of stress (FIG. 33c) (mean±SEM=0.653±0.197 versus 1.277±0.151; n=3), indicating that A3G-KD HuT78 cells proliferated more under high density culture conditions. However, the number of viable cells in WT and A3G-KD HuT78 cells did not show any difference at 48 hours after recovery from stress when cultured under non-stress conditions (FIG. 33c). These results suggest that hypoxic stress in lymphoma cells suppresses proliferation in vitro and that A3G plays an important role in this suppression.

Discussion

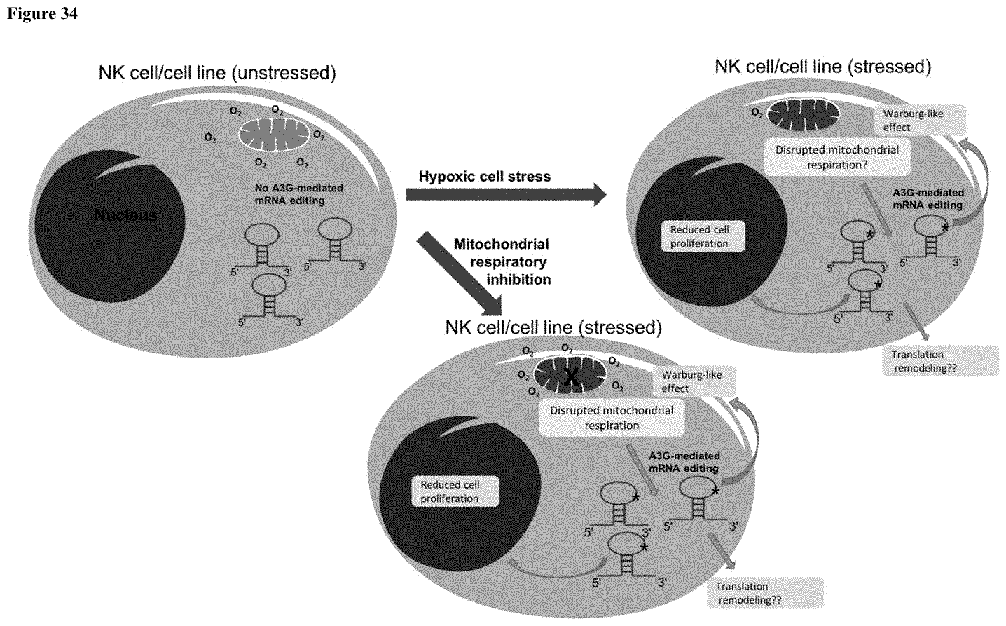

In this study we find that A3G edits scores of RNAs in NK cells and CD8+ T lymphocytes as well as lymphoma cell lines, when cultured at a high density and hypoxia. A3G-mediated site-specific RNA editing is triggered by the inhibition of mitochondrial respiration, and targets the mRNAs of many ribosomal and translational genes resulting in non-synonymous changes. A3G reduces mitochondrial respiration relative to glycolysis, and suppresses cell proliferation under stress in transformed lymphoma cells (FIG. 34). These results identify A3G cytidine deaminase as the third endogenous C>U RNA editing enzyme in mammals and together with A3A in myeloid cells, defines a new functional category of RNA editing enzymes that are active in immune cells. In addition, our findings uncover a previously unrecognized gene regulation mechanism in NK and CD8+ T cells that is induced by hypoxic stress.

There are two major differences in A3-mediated RNA editing and ADAR- and APOBEC1-mediated editing. First, A3-mediated RNA editing is induced upon hypoxic stress (A3A and A3G) or by IFNs (A3A), while it is essentially absent or rare in baseline unstressed immune cells (FIG. 29d). In contrast, ADAR and APOBEC1-mediated RNA editing events are considered to occur in baseline unstimulated cells. Second, A3-mediated RNA editing events occur in exonic coding regions of genes as commonly as in UTRs (FIG. 30c), whereas ADAR- and APOBEC1-mediated RNA editing events preferentially occur in UTRs, where they are at least an order of magnitude more frequent relative to coding exons. Together, these findings suggest that A3-mediated RNA editing plays a role in response to certain cell stress by altering protein function.

We find that the knockdown of A3G in HuT78 lymphoma cells reduces the predicted deleterious RNA editing of EIF3I in association with reduced mitochondrial respiration and cell proliferation during hypoxic stress. Thus, our findings indicate that A3G promotes hypoxic stress responses via RNA editing of EIF3I, ribosomal/translational genes and possibly other stress-related genes.

Cancer cells switch to aerobic glycolysis even in the presence of a functional mitochondria and this phenomenon is termed the ‘Warburg effect’. However, the function of Warburg effect in tumor growth, proliferation and support of cellular biosynthetic programs is still inconclusive. In response to acute hypoxia, A3G-medited RNA editing in the WT cells may promote Warburg effect by preferring glycolysis over mitochondrial respiration and decreased translation, while limiting overall cellular proliferation.

Interestingly, even though normal B cells and plasma cells show low expression of A3G (FIG. 33a), we find that the highest expression levels are observed in neoplastic B and plasma cell lines derived from acute lymphoblastic leukemia, B-cell lymphoma, Burkitt lymphoma and multiple myeloma. Increased expression of A3G in many B-cell leukemia/lymphoma cell lines (FIG. 35a) supports the notion that it may play an oncogenic role by enhancing survival under oxygen-limiting conditions. It is considered that NK cell function is impaired in the tumor micro-environment or chronic infections due to multiple factors, including hypoxia. This may be achieved in part by A3G-mediated RNA editing resulting in the cellular remodeling during stress.

We also find that RNA editing by A3G can be induced by normoxic inhibition of mitochondrial respiration and occurs independently of HIF-1α stabilization (FIG. 32). Despite the lack of HIF-1α stabilization, however, we find that mitochondrial respiratory inhibition mimics hypoxia and induces RNA editing by A3G. Hypoxia may trigger A3G-mediated RNA editing downstream of a pathway activated by mitochondrial respiratory inhibition as a result of severe oxygen deprivation or respiratory inhibitors in normoxia (FIG. 34).

The unexpected discovery of RNA editing functions for A3A and A3G require reconsideration of the physiological functions of the A3 enzymes solely as anti-viral factors. Our findings suggest that the primary function of A3G in vivo may be cellular RNA editing to facilitate adaptation to mitochondrial hypoxic stress in lymphocytes.

This present disclosure study demonstrates endogenous inducible site-specific RNA editing activity of the A3G cytidine deaminase, and identifies its physiological function in human immune and transformed cells. Widespread RNA editing by A3G can facilitate cellular adaptation to hypoxic cell stress triggered by mitochondrial respiratory inhibition in primary cytotoxic lymphocytes and lymphoma cell lines. A3G is the third endogenous C>U RNA editing enzyme to be identified in mammals. In addition, our study uncovers a novel epitranscriptomic gene regulation mechanism in cytotoxic lymphocytes, specifically NK cells. APOBEC3 cytidine deaminases may define a new class of RNA editing enzymes that are induced in response to certain cell stress factors.

Methods

RNA Sequencing

RNAs (DNA-free) were extracted from NK cells of 3 donors subjected to normoxia and hypoxia treatments (6 samples total) using the Total RNA clean-up and concentration kit (Norgen Biotek) as per the manufacturer's instructions. RNA Libraries were prepared using the Illumina TruSeq RNA Exome protocol and kit reagents. RNA input for intact total RNA was 10 ng. RNA QC analysis by electrophoresis (2100 Expert, B.02.08.51648, Agilent Technologies, Inc.) showed RIN numbers of 9.6, 7.8, 6.4 for normoxic and 2.8, 9.4 and 2 for hypoxic samples. These RIN numbers showed evidence of RNA degradation. Therefore, for degraded RNA samples input amount was determined by calculating the percentage of RNA fragments >200 nt (DV200) by running the samples on an RNA ScreenTape (Agilent Technologies) and performing region analysis using the Tapestation Analysis Software. Based on the DV200 calculation of 52-85%, 40 ng was the input amount and was considered suitable for this protocol. Fragmentation of the RNA was performed on intact samples. First and second strand synthesis were preformed to generate double-stranded cDNA. The 3′ ends were adenylated and Illumina adapters were ligated using T-A ligation. PCR was performed to generate enough material for hybridization and capture. PCR products were validated for the correct sizing using D1000 Screentape (Agilent Technologies). 200 ng of each product was pooled together in 4-plex reactions for hybridization and capture. Two sequential rounds of hybridization and capture were performed using the desired Capture Oligo pool. A second round of PCR was done to generate sufficient libraries for sequencing. Final libraries were validated for correct size distribution on a D1000 Screentape, quantified using KAPA Biosystems qPCR kit, and the 4-plex capture pools were pooled together in an equimolar fashion, following experimental design criteria.

Each pool was denatured and diluted to 2.4 pM with 1% PhiX control library added. Each pool was denatured and diluted to 16 pM for On-Board Cluster Generation and sequencing on a HiSeq2500 sequencer using 100 cycle paired-end cluster kit and rapid mode SBS reagents following the manufacturer's recommended protocol (Illumina Inc.) and 100 million paired reads per sample were obtained.

RNA Editing Bioinformatics Analysis

RNA editing events detection: Sequence reads passing quality filter from Illumina RTA were first checked using FastQC and then mapped to GENCODE (gencodegenes.org/) annotation database (V25) and human reference genome (GRCh38.p7) using Tophat2 with a lenient alignment strategy allowing at most 2 mismatches per read to accommodate potential editing events . The mapped bam files were further QCed using RSeqQC. Then all samples were run through the GATK best practices pipeline of SNV calling (https://gatkforums.broadinstitute.org/gatk/discussion/3892/the-gatk-best-practices-for-variant-calling-on-rnaseq-in-full-detail) using RNASeq data to obtain a list of candidate variant sites. All known SNPs from dbSNP (V144) were removed from further analyses.

Hypoxia induced editing events filtering: Pileups at candidate sites were generated using samtools for all samples and the base counts for alternative and reference base were calculated. Potential candidates for RNA editing were first filtered using the following two criteria: (a) at least 5% editing level on any sample within the population; (b) only C>T and G>A events were selected. The editing base counts were modeled as Binomial distribution and the effect of hypoxia on RNA editing at each site was tested with a generalized linear model (GLM) using paired samples. Multiple test adjustment was applied using Benjamini-Hochberg procedure to control false discovery rate (FDR). Hypoxia induced editing events were identified with log-odds-ratio greater than 0 and adjusted-p value less than 0.05.

Results: A table specifying the editing site, the type of editing event, editing level and number of reads on a reference and alternative bases on each sample for each group was initially produced filtering events with OR>1 and a FDR<0.05 level.

Annotation: Hypoxia induced editing events passing filters were annotated using ANNOVAR with RefSeq gene annotation database to identify gene features, protein changes and potential impact. Also 15 base pair upstream and downstream flanks from the variant sites were displayed in separate columns.

Manual filter: The above analyses initially revealed 383 C>U editing sites which were then subjected to a final stringent manual filtering step which retained only those sites (a) in exons and UTRs, (b) with -1 position (relative to edited C) is either a C or T and (c) within a stem-loop structure where the edited C is at the 3′-end of a putative tri- or tetra loop which is flanked by a stem that was at least 2 base pair long when base complementarity was perfect, or at least 4 base pair long when complementarity was imperfect by 1 nucleotide mismatch or 1 nucleotide bulging. This stringent manual filter reduced the number of edited sites to 122.

RNASeq Differential Expression Analysis

Raw counts for each gene were generated using HTSeq with intersection strict mode. Differential gene expression was analyzed by DESeq2. Bioconductor package with paired sample design to identify hypoxia induced gene expression changes.

Conservation analysis of amino acids recoded by RNA editing in NK cells The impact of non-synonymous RNA editing on protein function was examined by PolyPhen and SIFT programs from ENSEMBL VEP tool, which give a score and a verbal description of the impact (https://useast.ensembl.org/info/docs/tools/vep/index.html). In addition, conservation score based on 100 vertebrates basewise conservation was obtained from UCSC (phyloP100way).

Isolation and Culture of Cells

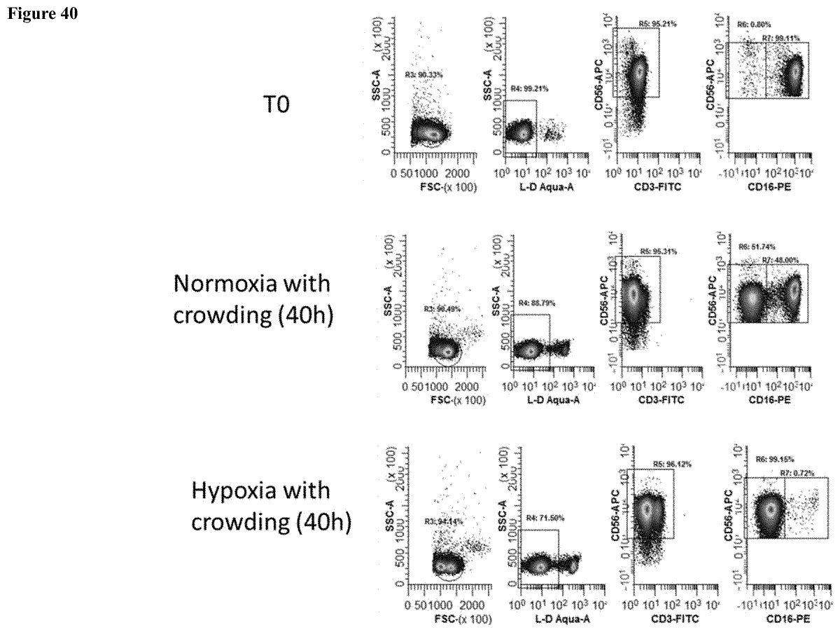

The HuT78, JVM3 and NK-92 cell-lines were obtained from ATCC. Hut 78 cells were cultured in IMDM (ATCC) containing 20% Fetal Bovine serum (FBS) (Sigma-Aldrich), JVM3 cells were cultured in RPMI (ATCC) containing 10% FBS and NK-92 cells were cultured in Alpha Minimum Essential medium without ribonucleosides and deoxyribonucleosides (Life Technologies) but with 2 mM L-glutamine and 1.5 g/L sodium bicarbonate as well as 0.2 mM inositol, 0.1 mM 2-mercaptoethanol, 0.02 mM folic acid, 500 U/ml IL-2 (Aldesleukin—a kind gift from Novartis), 12.5% horse serum (ATCC) and 12.5% FBS. Peripheral blood mononuclear cells (PBMCs) of anonymous platelet donors were isolated from peripheral blood in Trima Accel™ leukoreduction system chambers (Terumo BCT) in accordance with an institutional review board-approved protocol, in RPMI-1640 medium (Mediatech) with 10% FBS, 100 U/ml penicillin and 100 μg/m1 streptomycin (Mediatech). NK, CD4+ and CD8+ cells were isolated from PBMCs (cultured at 5×10⁷ in 1.8 ml per well in 12 well plates) by immunomagnetic negative selection using the EasySep™ Human NK Cell Isolation Kit (Stemcell Technologies, catalog #17955), EasySep™ Human CD4+ Cell Isolation Kit (Stemcell Technologies, catalog #17952) and EasySep™ Human CD8+ Cell Isolation Kit (Stemcell Technologies, catalog #17953), respectively, following the manufacturer's instructions. Enrichment for NK cells was >90% (Additional file 1; FIG. 40) and that of CD4+ and CD8+ was >99%, as verified by flow cytometry.

Cell Stress and Inhibitor Treatment

For cell crowding experiments, the HuT78 cells were cultured at a density of 0.5-1×10⁶ cells per 100 μl per well in 96 well plates for 22-24 hours at 37° C.

For hypoxia treatment, PBMCs were cultured at a density of 5×10⁷ in 1.8 ml per well in 12 well plates under 1% O₂, 5% CO₂ and 94% N₂ in an Xvivo™ System (Biospherix) for 40 hours. Following culture, NK, CD4+ and CD8+ cells were separated as mentioned above. In case of HuT78, the cells were cultured in the hypoxia chamber for 24 or 40 hours at a density of 1×10⁶ cells per ml in 6 well plates.

For testing the mitochondrial inhibitors, HuT78 and NK-92 cells were cultured at 0.533 10⁶ cells per 0.5 ml in 24 well plates in normoxia with or without AtA5 and MXT or hypoxia alone for 2 days at 37° C.

Human IFN-γ was obtained from PeproTech and used at a concentration of 50 ng/ml. AtA5 (Cayman chemical #11898) and MXT (Sigma Aldrich #T5580) was used at a concentration of 1 μM.

Extracellular Flux Assays

HuT78 cells (scramble WT and KD) were plated in 96-well plates at a density of 0.5 or 1×10⁶ in 100 μl per well (total 3×10⁶ cells) and incubated for 22-24 hours at 37° C. The cells were harvested and washed with PBS and re-counted on a hemocytometer (INCYTO C-Chip). Half of the cells were re-suspended in the XF base media specific for the Mitochondrial and the other half in XF base media specific for the Glycolytic Stress Tests (below), respectively. For all extracellular flux assays, cells were plated on cell-tak coated Seahorse XF96 cell culture microplates in (duplicate, triplicate or quadruplicate, depending on the cell count post culture) at a density of 3-6×10⁵ cells per well. The assay plates were spin seeded for 5 minutes at 1,000 rpm and incubated at 37° C. without CO₂ prior to performing the assay on the Seahorse Bioscience XFe96 (Agilent). The Mitochondrial Stress Test was performed in XF Base Media containing 10 mM glucose, 1 mM sodium pyruvate, and 2 mM L-glutamine and the following inhibitors were added at the final concentrations: Oligomycin (2 μM), Carbonyl cyanide 4-(trifluoromethoxy)phenylhydrazone (FCCP) (2 μM), Rotenone/Antimycin A (0.5 μM each). The Glycolytic Stress Test was performed in XF Base Media containing 2 mM L-glutamine and the following reagents were added at the final concentrations: Glucose (10 mM), Oligomycin (2 μM), and 2-deoxy-glycose (50 mM).

shRNA-Mediated Knock-Down of APOBEC3G in HuT78 Cells

A3G knock-down in Hut78 cells was performed at the RPCCC gene modulation shared resource. For A3G knock-down, GIPZ human A3G shRNAs with the following Clone ID's were used: V2LHS_80856, V2LHS_80785, V2LHS_80786 (Dharmacon). Lentiviruses were produced by cotransfection of 293T cells with A3G shRNA (or pGIPZ non-silencing control) along with psPAX2 and pMD2.G packaging plasmids, using the LipoD293 reagent (1:2.5 DNA to lipoD293 ratio) (SignaGen Laboratories) as per the manufacturer's instructions. Culture supernatants were collected 48 and 72 hours after transfection and cleared by filtration through 0.45 μm cellulose acetate syringe filter. For shRNA expression, 1×10⁶ Hut78 cells were pelleted and re-suspended with 1 ml culture supernatants containing the virus and 1 μl of 4 mg/ml polybrene. The cells were placed in 6 well plates and incubated for 30 mins at 37° C. The plate was sealed and spun at 1800 rpm for 45 mins in a microtiter rotor (Beckman Coulter) at room temperature and then incubated for 6 hours at 37° C. After infection the cells were centrifuged at 500 g for 5 mins and resuspended in IMDM media and incubated for 48 hours at 37° C. Puromycin (1 μg/ml) was added to the media to select for GFP positive cells. Clone ID V2LHS_80856 cells did not proliferate. Clone IDs V2LHS_80785 and V2LHS_80786 HuT78 cells were further sorted by the BD FACSaria II cell sorter (BD Biosciences) to obtain >95% pure GFP positive cells. A3G knock-down was verified by measuring the expression of A3G by qPCR. While Clone ID V2LHS_80785 did not show any difference in A3G gene expression in the WT and KD cells, clone ID V2LHS_80786 showed a significant reduction in A3G expression and was henceforth used for our studies (KD HuT78 cells) (FIG. 35c).

RT-PCR and Sanger Sequencing

Total RNA was isolated and reverse transcribed to generate cDNAs. DNA primers used for PCR were obtained from Integrated DNA Technologies and are noted in Table 6. Primers used for PCR of cDNA templates were designed such that the amplicons spanned multiple exons. Agarose gel electrophoresis of PCR products was performed to confirm the generation of a single product in a PCR and then sequenced on the 3130 xL Genetic Analyzer (Life Technologies) at the RPCCC genomic core facility as described previously (Sharma et al., Sci Rep 2016, 6:39100). To quantify RNA editing level, the major and minor chromatogram peak heights at putative edited nucleotides were quantified with Sequencher 5.0/5.1 software (Gene Codes, MI). Since the software identifies a minor peak only if its height is at least 5% that of the major peak's, we have considered 0.048 [=5/(100+5)] as the detection threshold. For quantitative PCR to assess APOBEC3G and APOBEC3F gene expression, reactions using LightCycler™ 480 Probes Master and SYBR™ Green I dye were performed on a LightCycler™ 480 System (Roche). Quantification cycle (C_{q}) values were calculated by the instrument software using the maximum second derivative method, and the mean C_{q} value of duplicate PCR reactions was used for analysis.

Immunoblotting Assays of Cell Lysates

Whole cell lysates were prepared and immunoblot was performed as described previously (Sharma et al., Nat Commun 2015, 6:6881; Sharma et al., Hum Mol Genet 2017, 26:1328-1339). APOBEC3G antiserum (Apo C17, catalog number-10082) was obtained from the NIH AIDS Reagent program (Kao et al., Retrovirology 2004, 1:27; Khan et al., J Virol 2005, 79:5870-5874), Rabbit monoclonal Phospho-eIF-2α (Ser51) (product number-3398, DG98) was obtained from Cell Signaling Technology, mouse monoclonal anti-β-actin (product number AM4302, AC-15) was obtained from Life Technologies, mouse monoclonal anti-HIF1α (product number GTX628480, GT10211) and rabbit polyclonal anti-α-Tubulin (product number GTX110432) was obtained from GeneTex and used at dilutions recommended by their manufacturers in 5% milk, except Phospho-eiF-2α, which was diluted in 5% BSA. HRP-conjugated goat anti-mouse or anti-rabbit antibodies were purchased from Life Technologies and used at 1:2000 dilution followed by chemiluminescent detection of the proteins.

Cell Proliferation Assay

WT and KD HuT78 cells (1×10⁶ cells in 100 μl per well) were seeded in 96-well round-bottom plates and incubated covered in the culture medium for 22 hours in a 37° C. humidified hypoxia chamber (1% O₂) or 37° C. humidified culture chamber (21% O₂). Cell viability was determined using a WST-8 viability stain based colorimetric assay (Dojindo Molecular Technologies, Inc.). Plates were read at 450 nm on an Epoch2 microplate reader (Biotek) using the Gen5 software (Biotek).

Statistical Analysis

Statistical analysis was performed using GraphPad Prism (7.03). A3G expression levels and mean editing levels in different cell types (FIG. 29) were first determined to be significantly statistically different by 1-way ANOVA followed by the recommended multiple comparison tests. RNA editing level and cell proliferation differences between WT and KD Hut78 cells for each gene (FIG. 35e and FIG. 33c) were examined by multiple t tests using the Holm-Sidak method, with alpha=0.05. The effect of inhibitors on RNA editing was first determined to be statistically significant by 2-way (FIG. 32b) or 1-way (FIG. 32d) ANOVA followed by, multiple comparisons of the treatment means for day 1 and/or day 2 using the recommended Dunnett's multiple comparisons test. Respiration to glycolysis ratios (R/G) were calculated using basal respiration value for each well divided by the average glycolysis value of all wells for each experimental group (n=3 for WT and KD HuT78 cells). These ratios were then normalized to the corresponding WT and KD TO (unstressed cells) ratios within each experimental group, which are set to 1 (FIG. 33b). The comparison of WT and KD HuT78 cells R/G ratios under stress, across all experiments were performed by Mann-Whitney non-parametric test after normalizing the R/G values against the average of WT stress ratio in experiment 1. P values are indicated by stars: *=p<0.05, **32 p<0.01, ***=p<0.001, ****=p<0.0001.

Others

Gene expression analysis of A3G is performed on two online platforms: (1) BIOGPS at biogps.org/#goto=welcome, a collection of thousands of gene expression datasets and (2) Cancer Cell Line Encyclopedia (CCLE) portal at portals.broadinstitute.org/ccle. CCLE database contains 1457 cell lines. Weblogo is created at weblogo.berkeley.edu/(2/19/18) with default parameters.

TABLE 5
Conservation of amino acids recoded by A3G-mediated RNA editing in translational and ribosomal genes

| Mutation | Gene | AAChange | PolyPhen | SIFT | PhyloP |
|---|---|---|---|---|---|
| 1:32231146-CT | EIF3I | NM_003757:exon11:c.C928T:p.R310C | Possibly damaging | deleterious(0) | 2.64453 |
| | | | (0.901) | | |
| 15:55196824-GA | RSL24D1 | NM_016304:exon1:c.C67T:p.R23C | Benign | Deleterious | 3.43396 |
| | | | (0.011) | (0.02) | |
| 6:35470271-CT | RPL10A | NM_007104:exon5:c.C403T:p.P135S | possibly_damaging | Deleterious | 7.64955 |
| | | | (0.866) | (0.01) | |
| 19:17863201-CT | RPL18A | NM_000980:exon5:c.C469T:p.R157W | Benign | Deleterious | 3.41186 |
| | | | (0.12) | (0.03) | |
| 17:28720820-CT | RPL23A | NM_000984:exon2:c.C139T:p.R47W | Benign | Tolerated | 1.92739 |
| | | | (0.013) | (0.28) | |
| 19:39433341-GA | RPS16 | NM_001020:exon5:c.C373T:p.R125C | possibly_damaging | Deleterious | 7.59689 |
| | | | (0.901) | (0.03) | |
| 16:1962610-GA | RPS2 | NM_002952:exon6:c.C596T:p.P199L | possibly_damaging | Deleterious | 9.862 |
| | | | (0.905) | (0.03) | |
| 1:39561754-GA | PABPC4 | NM_001135653:exon15:c.C1927T:p.H643Y | Benign | Deleterious | 10.003 |
| | | | (0.063) | (0.02) | |

TABLE 6

| RT-PCR | | | CDNA | GDNA |
|---|---|---|---|---|
| primers* | Forward | Reverse | SIZE | SIZE |
| Gene | (5′-to-3′) | (5′-to-3′) | (bp)# | (bp)# |
| EIF3I | ATGAGAGTGGA | GTGGTGGGATT | 536 | 4099 |
| | GAGCTCAACC | ACAGGCATGA | | |
| | (SEQ ID | (SEQ ID | | |
| | NO: 103) | NO: 104) | | |
| FAM89B | TCAACCTCGAC | GATGTGGAAGG | 321 | 713 |
| | TCAGCGCTG | CATCCTGCA | | |
| | (SEQ ID | (SEQ ID | | |
| | NO: 105) | NO: 106) | | |
| GOLGA5 | TCCAAAGATCC | CTCCTGCTCTC | 353 | 3423 |
| | AAAGAGACTCA | TCTTCAGAGTG | | |
| | AG(SEQ ID | (SEQ ID | | |
| | NO: 107) | NO: 108) | | |
| HSD17B10 | CAGCGTGTCGG | AGTCGGCTAGG | 564, | 2829 |
| | AGCGTGAAG | GAAGGGCACT | 646, | |
| | (SEQ ID | (SEQ ID | 673 | |
| | NO: 109) | NO: 110) | | |
| RFX7 | GCAATGGAGTG | TTGGAGAGCGA | 330 | 1790 |
| | ACTAATCTTCC | ATGGTGAGTG | | |
| | T(SEQ ID | (SEQ ID | | |
| | NO: 111) | NO: 112) | | |
| RPL10A | TTTCCGGTTAG | AAGGCCCGGAC | 638, | 636, |
| | CGCGGCGTGAG | ATTCTGC | 991 | 2300 |
| | AAGC(SEQ ID | (SEQ ID | | |
| | NO: 113) | NO: 114) | | |
| RPS2 | CGTCACCAAGT | GGGCTGAGGTG | 736 | 2334 |
| | TGGGCCGCTT | TAGCAGTCATC | | |
| | (SEQ ID | (SEQ ID | | |
| | NO: 115) | NO: 116) | | |
| TM7SF3 | GACAGTTACTG | CTAGGGTCCAG | 263 | 1531 |
| | GTCCACAAGC | AATGTTTGTCA | | |
| | (SEQ ID | C(SEQ ID | | |
| | NO: 117) | NO: 118) | | |
| TUFM | GGTCGGGACCC | ATGGCCAGCGT | 650 | 1543 |
| | TGAGTTAGG | GTTGGTGACT | | |
| | (SEQ ID | (SEQ ID | | |
| | NO: 119) | NO: 120 | | |
| APOBEC3G | GGCCGAGGACC | TTCTGACACAG | 99 | |
| | CGAAGG | GCTGCGAAG | | |
| | (SEQ ID | (SEQ ID | | |
| | NO: 121) | NO: 122) | | |
| APOBEC3F | TGCCTTGGTAC | AGTGGAAGTAG | 108 | |
| | AAATTCGATG | AATATGTGTGG | | |
| | AC(SEQ ID | ATAC(SEQ ID | | |
| | NO: 123) | NO: 124) | | |
| B2- | CTCGCTCCGTG | TGGATGAAACC | 152 | |
| Micro | GCCTTAGCT | CAGACACATAG | | |
| glubin | (SEQ ID | CA(SEQ ID | | |
| | NO: 125) | NO: 126) | | |

*underlined primers were used for sequencing

While the invention has been described through certain embodiments, routine modifications to the disclosure will be apparent to those skilled in the art, and such modifications are intended to be within the scope of the disclosure.

What is claimed is:

1. A method of inhibiting the growth of cancer cells comprising contacting the cancer cell with an agent which inhibits the expression of the gene, or the activity of, apolipoprotein B editing catalytic 3G (APOBEC3G).

2. The method of claim 1, wherein the agent is a polynucleotide.

3. The method of claim 2, wherein the polynucleotide is siRNA or shRNA.

4. The method of claim 1, wherein the cancer is a lymphoma.

5. A method for identifying agents that enhance or inhibit C>U deamination in a RNA molecule comprising:
a) providing a RNA substrate which contains a motif that contains a C that can undergo deamination to U;
b) contacting the RNA substrate with a apolipoprotein B editing catalytic (APOBEC) protein in the presence or absence of test agents;
c) determining the extent of C>U deamination and identifying agents in the presence of which either an increase or decrease of deamination is observed as compared to deamination in the absence of the agent,
wherein, the APOBEC protein is APOBEC3G.

6. The method of claim 5, wherein the motif is CCACCG.

7. The method of claim 5, wherein APOBEC3G is a purified protein.

8. The method of claim 5, wherein APOBEC3G is a recombinant protein.

9. The method of claim 5, wherein APOBEC3G is in a cell lysate.

10. A method for identifying agents that enhance or inhibit C>U deamination in a RNA substrate comprising:
a) providing cells which express apolipoprotein B editing catalytic 3G (APOBEC3G);
b) in the presence or absence of test agents, exposing the cells to conditions under which the cells can carry out APOBEC3G driven C>U deamination of RNA; and
c) determining the extent of C>U deamination in RNA to identify agents that induce or inhibit C>U deamination in RNA,
wherein an increase in C>U deamination as compared to deamination in the absence of the agent identifies an agent that enhances C>U deamination, and a decrease in C>U deamination as compared to deamination in the absence of the agent identifies an agent that inhibits C>U deamination.

11. The method of claim 10, wherein the cells are monocytes, and the condition under which the cells carry out APOBEC3G driven C>U deamination of RNA comprise hypoxia, exposure to interferon or both.

12. The method of claim 11, wherein the interferon in type 1 interferon or interferon gamma.

13. The method of claim 9, wherein the cells are macrophages, and the condition under which the cells carry out APOBEC3G driven C>U deamination of RNA comprises exposure to interferon.

14. The method of claim 13, wherein the interferon in type 1 interferon or interferon gamma.

15. A method for identifying agents that enhance or inhibit C>U deamination in a RNA substrate comprising:
a) providing cells which have been transfected to overexpress apolipoprotein B editing catalytic 3G (APOBEC3G);
b) in the presence or absence of test agents, determining the extent of C>U deamination in RNA to identify agents that enhance or inhibit C>U deamination in RNA.

16. The method of claim 15, wherein the cells are 293T cells.


SEQUENCE LISTING (126 sequences)

SEQ ID NO: 1 - DNA, 21 bp, Artificial Sequence
  siRNA
gaccuaccugugcuacgaatt

SEQ ID NO: 2 - DNA, 21 bp, Artificial Sequence
  siRNA
gcaguaugcucccgaucaatt

SEQ ID NO: 3 - DNA, 18 bp, Artificial Sequence
  primer
ggaattcggcacgaggac

SEQ ID NO: 4 - DNA, 20 bp, Artificial Sequence
  primer
ggtcctcagtggatgtaggc

SEQ ID NO: 5 - DNA, 21 bp, Artificial Sequence
  primer
tggactgcagatactgctgct

SEQ ID NO: 6 - DNA, 18 bp, Artificial Sequence
  primer
ttgccggccacaaccctt

SEQ ID NO: 7 - DNA, 20 bp, Artificial Sequence
  primer
agccttgtctgggtcccatc

SEQ ID NO: 8 - DNA, 17 bp, Artificial Sequence
  primer
agcctcgcctttgccga

SEQ ID NO: 9 - DNA, 20 bp, Artificial Sequence
  primer
tgacatcccccgcatcctgg

SEQ ID NO: 10 - DNA, 26 bp, Artificial Sequence
  primer
cacacatattcacttccaactttaac

SEQ ID NO: 11 - DNA, 17 bp, Artificial Sequence
  primer
ggccgaggacccgaagg

SEQ ID NO: 12 - DNA, 19 bp, Artificial Sequence
  primer
atgtccgcgcagaacagaa

SEQ ID NO: 13 - DNA, 22 bp, Artificial Sequence
  primer
ctttgacgagactctacagaag

SEQ ID NO: 14 - DNA, 20 bp, Artificial Sequence
  primer
aggcaggaagacctggcaga

SEQ ID NO: 15 - DNA, 21 bp, Artificial Sequence
  primer
aaacctgcagatgaccaagac

SEQ ID NO: 16 - DNA, 20 bp, Artificial Sequence
  primer
agagagagcaccatttactg

SEQ ID NO: 17 - DNA, 21 bp, Artificial Sequence
  primer
ctcacccaggaggggagaatc

SEQ ID NO: 18 - DNA, 22 bp, Artificial Sequence
  primer
gcaaaagaagttaacagctgag

SEQ ID NO: 19 - DNA, 20 bp, Artificial Sequence
  primer
tcaacctcgactcagcgctg

SEQ ID NO: 20 - DNA, 18 bp, Artificial Sequence
  primer
ttgttgtcagaggcccca

SEQ ID NO: 21 - DNA, 22 bp, Artificial Sequence
  primer
ggaagatcatcagtcaaggaag

SEQ ID NO: 22 - DNA, 20 bp, Artificial Sequence
  primer
tggctgctaccccactcctg

SEQ ID NO: 23 - DNA, 20 bp, Artificial Sequence
  primer
aatcggctggcgcaacgtca

SEQ ID NO: 24 - DNA, 20 bp, Artificial Sequence
  primer
ctcagctccagtggaaccag

SEQ ID NO: 25 - DNA, 21 bp, Artificial Sequence
  primer
cacctgcaagctctatgccat

SEQ ID NO: 26 - DNA, 24 bp, Artificial Sequence
  primer
aaatccatctggcataaatgatga

SEQ ID NO: 27 - DNA, 20 bp, Artificial Sequence
  primer
gggaatgctgctggagatag

SEQ ID NO: 28 - DNA, 18 bp, Artificial Sequence
  primer
tggtaccaggcctccaag

SEQ ID NO: 29 - DNA, 23 bp, Artificial Sequence
  primer
gatgatgttaagaagttcaaacc

SEQ ID NO: 30 - DNA, 22 bp, Artificial Sequence
  primer
gaggacataactctagaatctg

SEQ ID NO: 31 - DNA, 22 bp, Artificial Sequence
  primer
gaaaatgcaagataagacgcag

SEQ ID NO: 32 - DNA, 19 bp, Artificial Sequence
  primer
gctgcacctcagcgacaac

SEQ ID NO: 33 - DNA, 21 bp, Artificial Sequence
  primer
tgtggatagtctggataagct

SEQ ID NO: 34 - DNA, 20 bp, Artificial Sequence
  primer
tgattacctagaccgagggc

SEQ ID NO: 35 - DNA, 20 bp, Artificial Sequence
  primer
atagcagtggcctgagaaag

SEQ ID NO: 36 - DNA, 20 bp, Artificial Sequence
  primer
atctcaggagaatcgttggt

SEQ ID NO: 37 - DNA, 19 bp, Artificial Sequence
  primer
ggcgatgactcggacccag

SEQ ID NO: 38 - DNA, 20 bp, Artificial Sequence
  primer
tgtgacatcgtaaacatgag

SEQ ID NO: 39 - DNA, 22 bp, Artificial Sequence
  primer
ccagactccgattttgatggag

SEQ ID NO: 40 - DNA, 22 bp, Artificial Sequence
  primer
gcagaagaatggtacaaatcca

SEQ ID NO: 41 - DNA, 19 bp, Artificial Sequence
  primer
gcatcagcaagagcaacag

SEQ ID NO: 42 - DNA, 15 bp, Artificial Sequence
  primer
ctggtgcctggggcg

SEQ ID NO: 43 - DNA, 21 bp, Artificial Sequence
  primer
catgtgctggtcattgagcag

SEQ ID NO: 44 - DNA, 18 bp, Artificial Sequence
  primer
gtccaggcgctccacttc

SEQ ID NO: 45 - DNA, 20 bp, Artificial Sequence
  primer
ttctgacacaggctgcgaag

SEQ ID NO: 46 - DNA, 21 bp, Artificial Sequence
  primer
caacttcatcctgaatctcct

SEQ ID NO: 47 - DNA, 21 bp, Artificial Sequence
  primer
aaagagcacgcagaggtccag

SEQ ID NO: 48 - DNA, 20 bp, Artificial Sequence
  primer
gggtacagtgcagacgaatc

SEQ ID NO: 49 - DNA, 21 bp, Artificial Sequence
  primer
agtcatcttttggcttggaag

SEQ ID NO: 50 - DNA, 20 bp, Artificial Sequence
  primer
atatcccaggagtacaccca

SEQ ID NO: 51 - DNA, 23 bp, Artificial Sequence
  primer
cgatagcaattgccctgaaatcc

SEQ ID NO: 52 - DNA, 21 bp, Artificial Sequence
  primer
gttggttgtccagcagtgact

SEQ ID NO: 53 - DNA, 20 bp, Artificial Sequence
  primer
gatgtggaaggcatcctgca

SEQ ID NO: 54 - DNA, 20 bp, Artificial Sequence
  primer
tccttccgcctgagcttctt

SEQ ID NO: 55 - DNA, 22 bp, Artificial Sequence
  primer
cccaagtatgatcaagaatagc

SEQ ID NO: 56 - DNA, 21 bp, Artificial Sequence
  primer
gccctatttgctggatcatcc

SEQ ID NO: 57 - DNA, 20 bp, Artificial Sequence
  primer
ctcgtaggtcttcaccatcc

SEQ ID NO: 58 - DNA, 20 bp, Artificial Sequence
  primer
aggatcccgttcaccatcac

SEQ ID NO: 59 - DNA, 19 bp, Artificial Sequence
  primer
caaggggagcagcagaagg

SEQ ID NO: 60 - DNA, 20 bp, Artificial Sequence
  primer
ctccatttcctgccttagcc

SEQ ID NO: 61 - DNA, 24 bp, Artificial Sequence
  primer
tcaaaccttgagttgaattccata

SEQ ID NO: 62 - DNA, 20 bp, Artificial Sequence
  primer
ctgcggtggtagtcgttgtc

SEQ ID NO: 63 - DNA, 22 bp, Artificial Sequence
  primer
atcttgcttcctcttgagtgca

SEQ ID NO: 64 - DNA, 22 bp, Artificial Sequence
  primer
gatttatgtctcctcttcttac

SEQ ID NO: 65 - DNA, 20 bp, Artificial Sequence
  primer
ctggtcctggagtttctgga

SEQ ID NO: 66 - DNA, 19 bp, Artificial Sequence
  primer
ctgagcctggagctggggt

SEQ ID NO: 67 - DNA, 22 bp, Artificial Sequence
  primer
gtcaaagtagagtcaacttcat

SEQ ID NO: 68 - DNA, 19 bp, Artificial Sequence
  primer
gcacatgctccttggtcca

SEQ ID NO: 69 - DNA, 22 bp, Artificial Sequence
  primer
atgccatcagcaagaggtttgt

SEQ ID NO: 70 - DNA, 20 bp, Artificial Sequence
  primer
ctctgacaccaggtgcatgg

SEQ ID NO: 71 - DNA, 22 bp, Artificial Sequence
  primer
agcagactgtgaagaagctgct

SEQ ID NO: 72 - DNA, 20 bp, Artificial Sequence
  primer
ccaaattcacccaagaagag

SEQ ID NO: 73 - DNA, 22 bp, Artificial Sequence
  primer
ggaatgtaccactcatatgaag

SEQ ID NO: 74 - DNA, 21 bp, Artificial Sequence
  primer
gctcttctttcctcaggagtg

SEQ ID NO: 75 - DNA, 21 bp, Artificial Sequence
  primer
ctgtaggtggcaatctcaatg

SEQ ID NO: 76 - DNA, 21 bp, Artificial Sequence
  primer
tcagtacttcttgagccattc

SEQ ID NO: 77 - DNA, 23 bp, Artificial Sequence
  primer
gcaagagttccttttcatcttgc

SEQ ID NO: 78 - DNA, 23 bp, Artificial Sequence
  primer
ctttcattataaacccgctatag

SEQ ID NO: 79 - DNA, 20 bp, Artificial Sequence
  primer
tcaccctgtgtcctttgcag

SEQ ID NO: 80 - DNA, 22 bp, Artificial Sequence
  primer
gaagactatttgctttccctgg

SEQ ID NO: 81 - DNA, 23 bp, Artificial Sequence
  primer
tatgcatgatttaccatctttgc

SEQ ID NO: 82 - DNA, 19 bp, Artificial Sequence
  primer
gttggctttggtccaccac

SEQ ID NO: 83 - DNA, 21 bp, Artificial Sequence
  primer
ctttctcttgccacagcagct

SEQ ID NO: 84 - DNA, 23 bp, Artificial Sequence
  primer
ccagcaaaatggaattatcttgt

SEQ ID NO: 85 - DNA, 20 bp, Artificial Sequence
  primer
gcactcagctcactgtgctt

SEQ ID NO: 86 - DNA, 20 bp, Artificial Sequence
  primer
cagttggtccctttttcagc

SEQ ID NO: 87 - DNA, 21 bp, Artificial Sequence
  primer
tgcattgtttattcctcaggc

SEQ ID NO: 88 - DNA, 20 bp, Artificial Sequence
  primer
caccagacatctttctcacc

SEQ ID NO: 89 - DNA, 21 bp, Artificial Sequence
  primer
gtcatgggatcagtggcttac

SEQ ID NO: 90 - DNA, 19 bp, Artificial Sequence
  primer
ggaggggattcttgctcac

SEQ ID NO: 91 - DNA, 21 bp, Artificial Sequence
  primer
tgaaaatagccacacatacgg

SEQ ID NO: 92 - DNA, 21 bp, Artificial Sequence
  primer
ctctccttcaatagctggctt

SEQ ID NO: 93 - DNA, 21 bp, Artificial Sequence
  primer
aacacatgccatcacaatgcc

SEQ ID NO: 94 - DNA, 21 bp, Artificial Sequence
  primer
gctttgtaaaagcaactgggt

SEQ ID NO: 95 - DNA, 21 bp, Artificial Sequence
  primer
acgcatccaagtctgagttcc

SEQ ID NO: 96 - DNA, 20 bp, Artificial Sequence
  primer
gcatccggacaggcatccaa

SEQ ID NO: 97 - DNA, 21 bp, Artificial Sequence
  primer
gccatgcggacctgcgcttct

SEQ ID NO: 98 - DNA, 21 bp, Artificial Sequence
  primer
ctggagccccagcttctcctg

SEQ ID NO: 99 - DNA, 26 bp, Artificial Sequence
  primer
tgattacgacgccctatataaggagg

SEQ ID NO: 100 - DNA, 19 bp, Artificial Sequence
  primer
ggccgtaaaagccacagag

SEQ ID NO: 101 - DNA, 21 bp, Artificial Sequence
  primer
gagatgaaccaagtgaccctg

SEQ ID NO: 102 - DNA, 18 bp, Artificial Sequence
  primer
tagatgcgggcagcgaag

SEQ ID NO: 103 - DNA, 21 bp, Artificial Sequence
  primer
atgagagtggagagctcaacc

SEQ ID NO: 104 - DNA, 21 bp, Artificial Sequence
  primer
gtggtgggattacaggcatga

SEQ ID NO: 105 - DNA, 20 bp, Artificial Sequence
  primer
tcaacctcgactcagcgctg

SEQ ID NO: 106 - DNA, 20 bp, Artificial Sequence
  primer
gatgtggaaggcatcctgca

SEQ ID NO: 107 - DNA, 24 bp, Artificial Sequence
  primer
tccaaagatccaaagagactcaag

SEQ ID NO: 108 - DNA, 22 bp, Artificial Sequence
  primer
ctcctgctctctcttcagagtg

SEQ ID NO: 109 - DNA, 20 bp, Artificial Sequence
  primer
cagcgtgtcggagcgtgaag

SEQ ID NO: 110 - DNA, 21 bp, Artificial Sequence
  primer
agtcggctagggaagggcact

SEQ ID NO: 111 - DNA, 23 bp, Artificial Sequence
  primer
gcaatggagtgactaatcttcct

SEQ ID NO: 112 - DNA, 21 bp, Artificial Sequence
  primer
ttggagagcgaatggtgagtg

SEQ ID NO: 113 - DNA, 26 bp, Artificial Sequence
  primer
tttccggttagcgcggcgtgagaagc

SEQ ID NO: 114 - DNA, 18 bp, Artificial Sequence
  primer
aaggcccggacattctgc

SEQ ID NO: 115 - DNA, 21 bp, Artificial Sequence
  primer
cgtcaccaagttgggccgctt

SEQ ID NO: 116 - DNA, 22 bp, Artificial Sequence
  primer
gggctgaggtgtagcagtcatc

SEQ ID NO: 117 - DNA, 21 bp, Artificial Sequence
  primer
gacagttactggtccacaagc

SEQ ID NO: 118 - DNA, 23 bp, Artificial Sequence
  primer
ctagggtccagaatgtttgtcac

SEQ ID NO: 119 - DNA, 20 bp, Artificial Sequence
  primer
ggtcgggaccctgagttagg

SEQ ID NO: 120 - DNA, 21 bp, Artificial Sequence
  primer
atggccagcgtgttggtgact

SEQ ID NO: 121 - DNA, 17 bp, Artificial Sequence
  primer
ggccgaggacccgaagg

SEQ ID NO: 122 - DNA, 20 bp, Artificial Sequence
  primer
ttctgacacaggctgcgaag

SEQ ID NO: 123 - DNA, 23 bp, Artificial Sequence
  primer
tgccttggtacaaattcgatgac

SEQ ID NO: 124 - DNA, 26 bp, Artificial Sequence
  primer
agtggaagtagaatatgtgtggatac

SEQ ID NO: 125 - DNA, 20 bp, Artificial Sequence
  primer
ctcgctccgtggccttagct

SEQ ID NO: 126 - DNA, 24 bp, Artificial Sequence
  primer
tggatgaaacccagacacatagca